(19) United States
(12) Patent Application Publication
(10) Pub. No.: US 2019/0264200 A1
(43) Pub. Date: Aug. 29, 2019

(54) Generic System of Antisense Oligonucleotide

(71) Applicants: Tao Chen, London (CA); Jinghan Li, Beijing (CN); Shangan Zhang, Beijing (CN)

(72) Inventors: Tao Chen, London (CA); Jinghan Li, Beijing (CN); Te-ming Chen, Beijing (CN); Shangan Zhang, Beijing (CN)

(21) Appl. No.: 15/697,353

(22) Filed: Sep. 6, 2017

Related U.S. Application Data
(62) Division of application No. 14/141,404, filed on Dec. 26, 2013.

(51) Int. Cl. C12N 15/10; C12N 15/11

(52) U.S. Cl. CPC C12N 15/1093; C12N 15/111; C12N 2310/14; C12N 2330/31; C12N 2310/11

(57) ABSTRACT

The present invention relates to the method of rational design for generic antisense oligonucleotide libraries for genome-wide screening, particularly in the research area for antisense pharmaceutical lead discovery and validation. The libraries are being constructed systematically. The building block is antisense codon. The present invention is aimed at overcoming the drawbacks of both oligonucleotide libraries constructed according to algorithm of 4.sup.n and randomized libraries at the same time. It is applicable for all cells, tissues, organs and organisms with necessary judgments.

This is a divisional application of U.S. Ser. No. 14/141,404 filed on Dec. 26, 2013, all of which is incorporated herein by reference in their entirety.

### COPYRIGHT NOTICE

Pursuant to 37 C.F.R. 1.71(e), the applicants notify that this patent document contains materials which are subject to copyright protection. The owners of the copyright have no objection to the facsimile reproduction of the document as it appears in the patent file in U.S. Patent and Trademark Office but otherwise reserve all copyright rights whatsoever.

### FIELD OF THE INVENTION

The present invention relates to the generation and construction of generic system antisense oligonucleotide for genome-wide screening.

### BACKGROUND OF THE INVENTION

### The Distinction Between Codon and Nucleotide

While nucleic acids consist of four nucleotides with four distinct bases: Adenine (A), Thymine (T)/Uracil (U), Guanine (G) and Cytosine (C) respectively, the coding sequences of genes are organized in codons which in turn code for specific amino acids. Codons are arranged in an oriented, consecutive, non-overlapping and linear manner with a unique starting and end point. In appearance, either nucleotides or codons could be used to measure a sequence of nucleic acids such as DNA and their corresponding transcripts such as mRNA. In nature, nucleotides are chemical compositions of DNA and codon. Genetic information is encoded in codon (FIG. 1). Each codon encodes for a specific essential amino acid (EAA) except the stop codons that terminate peptide synthesis. Therefore, codon is virtually the function unit of a gene, its corresponding transcript(s) such as mRNA(s) and its corresponding translation(s) such as peptide(s). Codons could enable the three major forms of product of a gene into a unique integrated system, which reflects the nature. Nucleotides are chemical compositions of a given gene and could not be used to do the same as codons. One ordinary skilled in the relevant art would recognize the distinction between codon and nucleotide concerning structure and function in both theory and practice. They are related but distinct from each other. When designing a genome-wide antisense oligonucleotide library, an antisense-codon-based design has the capacity to convert it precisely into either a corresponding sense oligonucleotide library or corresponding peptide library vice versa according to a specific problem(s) addressed. One ordinary skilled in the relevant art would recognize codon-based design is the core element of the present invention. It is one invention with multiple application aspects.

### The Distinction Between Nuclear Genome and Mitochondria Genome

It is known in the art that 64 codons (genetic code) consist of 64 nucleotide triplets. Many, if not most, 61 codons encode the 20 essential L-amino acids (EAA) and three other codons encode for peptide termination among the 64 codons. 5′-ATG/5′-AUG, 5′-GTG/5′-GUG, 5′-ATA/5′-AUA, 5′-TTG/5′-UUG, 5′-ACG/5′-ACG and 5′-CTG/5′-CUG may function as start codons in DNA and mRNA. For example, 5′-ATA/5′-AUA is the start codons for mammalian mitochondria. Whereas, 5′-ATG/5′-AUG is the major start codon for many life forms. It is similar to stop codons that many, if not most, 5′-TAA/5′-UAA, 5′-TGA/5′-UGA and 5′-TAG/5′-UAG are the three major stop codons in DNA and mRNA. Exceptions exist. For example, in mammalian mitochondrial, 5′-AGA and 5′-AGG are stop codons instead of coding for Arginine. There are four stop codons: 5′-AGA, 5′-AGG, 5′-TAA/5′-UAA and 5′-TAG/5′-UAG for mammalian mitochondria DNA and mRNA. In accordance with Watson-Crick DNA complementary rule, each of the four specific mammalian mitochondria antisense stop codons for DNA and mRNA was being produced and vice versa. 5′-TGA encode Tryptophan instead of the stop codon in mammalian mitochondria. Additionally, there are 60 specific codons that encode 20 EAA in mammalian mitochondria. The said 60 specific mammalian mitochondria codons for DNA and mRNA are as following:

5′-TTT/UUU, 5′-TTC/5′-UUC, 5′-TTA/5′-UUA, 5′-TTG/5′-UUG, 5′-CTT/5′-CUU, 5′-CTC/5′-CUC, 5′-CTA/5′-CUA, 5′-CTG/5′-CUG, 5′-ATT/5′-AUU, 5′-ATC/5′-AUC, 5′-ATA/5′-AUA, 5′-ATG/5′-AUG, 5′-GTT/5′-GUU, 5′-GTC/5′-GUC, 5′-GTA/5′-GUA, 5′-GTG/5′-GUG, 5′-TCT/5′-UCU, 5′-TCC/5′-UCC, 5′-TCA/5′-UCA, 5′-TCG/5′-UCG, 5′-CCT/5′-CCU, 5′-CCC/5′-CCC, 5′-CCA/5′-CCA, 5′-CCG/5′-CCG, 5′-ACT/5′-ACU, 5′-ACC/5′-ACC, 5′-ACA/5′-ACA, 5′-ACG/5′-ACG, 5′-GCT/5′-GCU, 5′-GCC/5′-GCC, 5′-GCA/5′-GCA, 5′-GCG/5′-GCG, 5′-TAT/5′-UAU, 5′-TAC/5′-UAC, 5′-CAT/5′-CAU, 5′-CAC/5′-CAC, 5′-CAA/5′-CAA, 5′-CAG/5′-CAG, 5′-AAT/5′-AAU, 5′-AAC/5′-AAC, 5′-AAA/5′-AAA, 5′-AAG/5′-AAG, 5′-GAT/5′-GAU, 5′-GAC/5′-GAC, 5′-GAA/5′-GAA, 5′-GAG/5′-GAG, 5′-TGT/5′-UGU, 5′-TGC/5′-UGC, 5′-TGA/5′-UGA, 5′-TGG/5′-UGG, 5′-CGT/5′-CGU, 5′-CGC/5′-CGC, 5′-CGA/5′-CGA, 5′-CGG/5′-CGG, 5′-AGT/5′-AGU, 5′-AGC/5′-AGC, 5′-GGT/5′-GGU, 5′-GGC/5′-GGC, 5′-GGA/5′-GGA and 5′-GGG/5′-GGG. In accordance with Watson-Crick DNA complementary rule, a corresponding complete set of 60 specific mammalian mitochondria antisense codons for DNA and mRNA was being produced and vice versa.

### Codon-Based Antisense, Sense and Expressed Oligonucleotide

In general, a gene includes transcribed and non-transcribed sequence regions. For a non-limiting example, a gene may contain non-transcribed enhancer or and promoter. For another non-limiting example, a gene may contain 5′-UTR, ORF, 3′-UTR and introns. For one another non-limiting example, a gene may contain non-coding RNAs, such as tRNA, rRNA, miRNA and piRNA. The invention envisions a coding region, such as ORF of a gene as a linear polymer selected from a group consisting of all possible combinations of 61 amino acid coding codons with a start codon at its 5′-end and a stop codon at its 3′-end. 61 amino acid coding codons are referred to 61 codons hereinafter. This is different from the traditional concept which perceives a gene as a linear DNA sequence selected from a group consisting of all combinations of four distinct nucleotide of A, T, G and C whether coding region, 5′-UTR or 3′-UTR. With the invention, any coding region, such as ORF is selected from a group consisting of all possible combinations of 61 codons with a start codon at its 5′-end and a stop codon at its 3′-end. A 5′-UTR is selected from a group consisting of all possible combinations of 64 codons with a start codon at its 3′-end. A 3′-UTR is selected from a group consisting of all possible combinations of 64 codons with a stop codon adding at its 5′-end. In accordance with Watson-Crick DNA complementary rule, a series of corresponding antisense-codon-based antisense oligonucleotides of ORF, 5′-UTR and 3′-UTR have been produced and vice versa (FIG. 1). In accordance with Central Dogma, a series of corresponding expressed-codon-based peptides of ORF have been produced either directly from mentioned sense oligonucleotides or indirectly from its corresponding antisense oligonucleotides and vice versa. Applying innovative concepts makes it possible to differentiate the genes of mammalian genomic DNA origin from mitochondrial genes. The genes of mammalian mitochondria possess unique characteristics: for example, 5′-ATA replaces 5′-ATG for Met.; 5′-TGA encodes Trp. instead of termination. Therefore, a given coding region of a given gene of mammalian mitochondria could be envisioned as one selected from the group of linear DNA sequences consisting of all possible combinations of 60 codons in which 5′-ATA substitutes 5′-ATG and 5′-TGA substitutes for 5′-AGA and 5′-AGG of the group of 61 codons. Such a linear DNA sequence has 5′-ATA at its 5′-end as the start codon and one of 5′-AGA, 5′-AGG, 5′-TAG and 5′-TAA at its 3′-end as stop codon. In accordance with Watson-Crick DNA complementary rule, a series of corresponding antisense-codon-based antisense oligonucleotides of mammalian mitochondria have been produced and vice versa. In accordance with Central Dogma, a series of corresponding expressed-codon-based peptides of mammalian mitochondria have been produced either directly from mentioned sense oligonucleotides or indirectly from its corresponding antisense oligonucleotides and vice versa. The invention envisions a gene product such as a peptide or polypeptide as a linear polymer selected from a group consisting of all possible combinations of 20 essential amino acids (EAA) with an amino acid encoded by a 5′-start codon, such as Methionine at its N-terminal. The 20 EAA are perceived as the expressed codons of the 61 codons in the view of this invention. In accordance with Central Dogma, a series of corresponding codon-based oligonucleotides of ORF have been produced from mentioned peptides and vice versa.

The citation of a reference herein and hereafter shall not be construed as an admission that such reference is prior art to the present invention.

### BRIEF DESCRIPTION OF THE DRAWINGS

FIG. 1 presents diagram of structures, functions, operation and correlations among template strand, non-template strand, Pre-mRNA, mRNA and peptide.

FIG. 2 presents diagram of correlations among antisense oligonucleotide, sense oligonucleotide, mRNA, first strand cDNA and second strand cDNA.

### DESCRIPTION OF THE INVENTION

It is known in the art that the selection of target sites is generally considered as an important element in construction of an antisense oligonucleotide library. 5′-UTR, 3′-UTR and initiation region of ORF may possess certain binding sites for regulatory proteins and peptides. In theory, to reduce possible spatial hindering effect, those regions may not give the priority when designing target sequence for siRNA except for antisense oligonucleotide. Therefore, the present invention includes regions of ORF, 5′-UTR and 3′-UTR for the target sites selection in generic genome-wide antisense screens.

### Target Site: ORF Sites

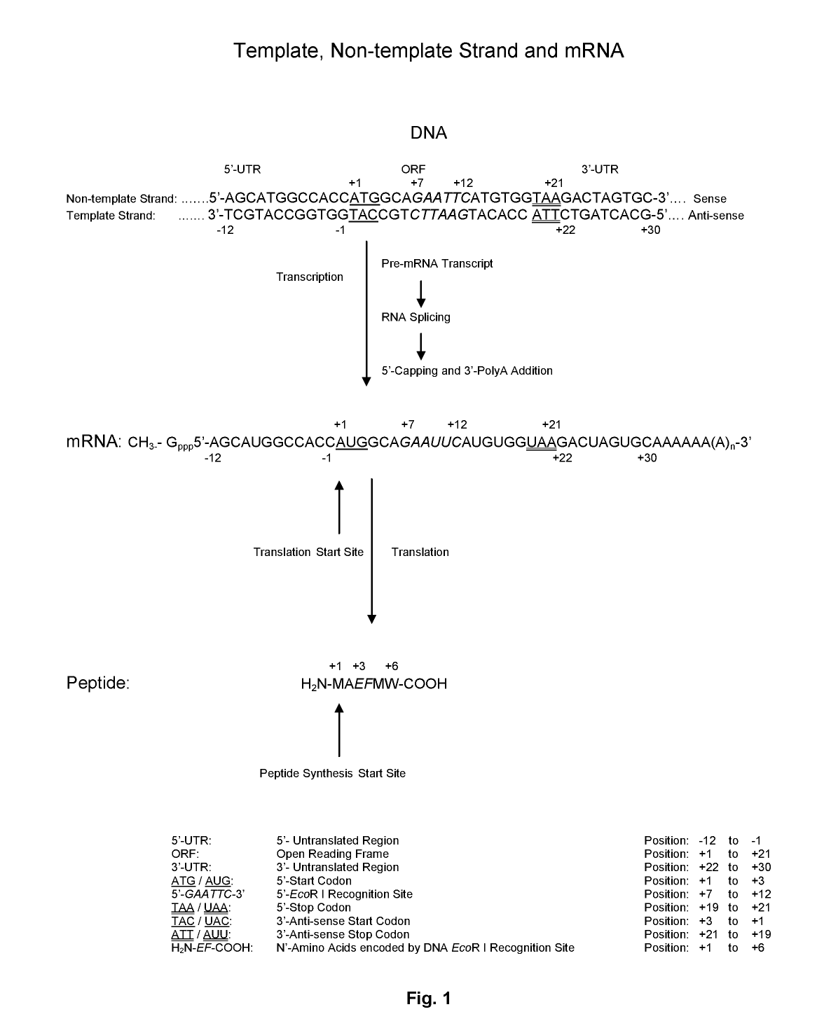

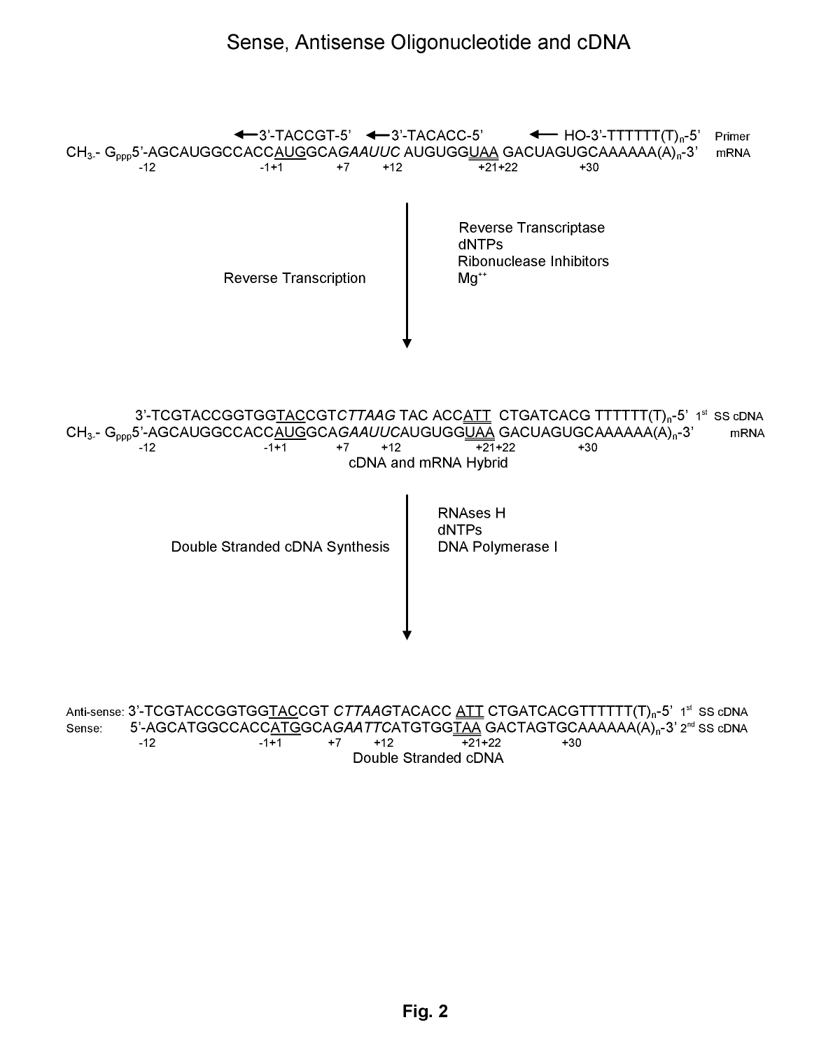

It is known in the art that ORF sequence is one of the preferred target areas for antisense compounds, particularly antisense oligonucleotides. Antisense oligonucleotides specifically designed to target sites around initiation codon of translation may interfere with the binding of ribosomes to mRNA. The interference in turn inhibits the translation of undesirable peptides. A 20-mer antisense oligonucleotide (PS-ODN, ISIS 2530) targeted the translation initiation sequence of H-ras mRNA. As a result, it selectively reduced the expression of H-ras protein in vitro (Chen et al., J. Biol. Chem. 271(45): 28259-28265, 1996). ORF refers to the sequence between the positions of a start codon and a stop codon. Although a specific coding region consists of a specific combination of a set of specific codons at a specific length, a given sequence with given length of ORF of a given gene or a given mRNA could be identified among the group of linear consecutive DNA or RNA sequences consisting of all possible combinations of 61 codons that encode 20 EAA. It is known in the art that genes of eukaryotic and prokaryotic species may have two or more alternative start codons, any one of which may be preferentially selected as a unique start codon in a specific tissue or cell or under a specific physical or pathological condition(s). At transcription level, eukaryotic pre-mRNA may require to be processed, edited, modified and transported prior translation. Splicing and editing belongs to mRNA post-transcriptional modification. In alternative splicing, pre-mRNA may be spliced into several different ways, allowing ORF of a given gene to encode multiple peptides. Sometimes, the editing process may bring forth an early stop codon which shortens the peptide translation. Nevertheless, once a start codon and a stop codon were determined for a given ORF or a corresponding mature transcription, such as mRNA, each 5′-terminal sequence of the given ORF or mRNA has a start codon at its 5′-end which could be chosen as the sequence of orientation of the given ORF or mRNA. Each 3′-terminal sequence of the given ORF or mRNA has a stop codon at its 3-end which could be chosen as the sequence of orientation of the given ORF or mRNA (FIG. 1). Thus, any and all terminal sequences of ORF or mRNA of a given length could be produced from either its 5′-end or 3′-end according to the genetic algorithm of 61.sup.(n−m) under conditions: n−m=1, or n−m>1, or n−m=2, or n−m=3, or n−m=4, or n−m=5, or n−m=6, or n−m=7, or n−m=8, or n−m=9, or n−m=10, n>m, n−m<infinity, neither n nor m is equal to zero, both n and m are integers, n is the unit of measurement of the length of ORF sequence, n represents the entire length of a given ORF or mRNA sequence measured by codon or expressed codon (essential amino acid), m represents the length of the sequence of orientation which is a selected sequence for the orientation of the entire sequence which is measured by codon or expressed codon (essential amino acid). For a non-limiting example, if 5′-AGC in 5′-AGCGCACTC is the sequence of orientation which is a selected sequence for the orientation of the entire sequence, then n=3, m=1, n−m=2. If n=3 and one 5′-AGc is at 5′-end, 3,721 distinct 5′-AGC oriented oligonucleotide sequences of three-codon-length-long could be produced according to algorithm of 61.sup.(n−m). The length of three-codon equals nine-nucleotide (9 mers). The complete collection of above 3,721 distinctive 9-mer 5′-AGC oriented oligonucleotide sequences has formed a 9-mer generic sense-codon-based DNA or and RNA oligonucleotide or and PCR primer library accordingly. The complete collection of above 3,721 distinctive 9-mer 5′-AGC oriented sense RNA oligonucleotide sequences has formed a 9-mer generic sense-codon-based RNA oligonucleotide library accordingly. In accordance with Watson-Crick DNA complementary rule, a corresponding 9-mer generic antisense-codon-based RNA oligonucleotide library could be produced and vice versa. To address a specific problem of gene expression and regulations, the above mentioned libraries, such as library comprising 9-mer sense-codon-based RNA oligonucleotides; its corresponding library comprising 9-mer antisense-codon-based RNA oligonucleotides could be used alone or and in combination (FIG. 1), (FIG. 2). Therefore, the above mentioned libraries could be integrated or and included into a singular product or and in one method.

5′-terminal sequence of ORF of a given gene or a given mRNA of a given length can be translated into a peptide sequence, which can be identified among the group of peptides of linear consecutive amino acids sequences consisting of all possible combinations of 20 (EAA) with a L-amino acid encoded by a start codon at its N-terminal having the same unit number(s) of length as the corresponding 5′-terminal sequence of ORF or mRNA. Methionine is encoded by 5′-ATG. The ordinary level of skill in the pertinent art would recognize that the 5′-ATG at 5′-end terminal of ORF or mRNA is the sequence of orientation. Thus, any and all N-terminal peptide sequences of a given length could be produced from its N-terminal(s) according to the genetic algorithm of 20.sup.(n−m) as well under conditions: n−m=1 or n−m>1, or n−m=2, or n-m=3, or n−m=4, or n−m=5, n>m, n−m<infinity, neither n nor m is equal to zero, both n and m are integers, n is the unit of measurement of the length of peptide, n represents the entire length of a given peptide sequence measured by EAA (expressed codon), m represents the length of the sequence of orientation which is a selected sequence for the orientation of the entire sequence which is measured by EAA (expressed codon). For example, if Methionine (M) in N-MKS is the sequence of orientation which is a selected sequence for the orientation of the entire sequence, then n=3 and m=1. If n=6 and one Methionine is at N-terminal (m=1), 3.2 million distinct N-Methionine oriented 6-EAA-length-long peptide sequences could be produced according to algorithm of 20.sup.(n−m). The complete collection of the above 3.2 million distinctive 6-EAA-length long peptide sequences has formed a generic hexa-expressed-codon-based peptide library/hexa-peptide library accordingly. In accordance with Central Dogma, a corresponding generic sense oligonucleotide probe library or a corresponding generic antisense oligonucleotide library could be produced and vice versa. To address a specific problem of gene expression and regulations, the above mentioned libraries such as sense-codon-based oligonucleotide library, antisense-codon-based oligonucleotide library and peptide library derived and deduced from a generic hexa-expressed-codon-based peptide library/hexa-peptide library could be used alone or and in combination (FIG. 1), (FIG. 2). Therefore, the above mentioned libraries could be integrated or and included into a singular product or and in one method.

3′-terminal sequence of ORF of a given gene or a given mRNA of a given length could be translated into peptide sequence, which could be identified among the group of peptides of linear consecutive amino acids sequences consisting of all possible combinations of 20 (EAA) having the same unit number(s) of the length as the corresponding 3′-end terminal sequence of ORF. Thus, any and all C-terminal peptide sequences of a given length could be produced from its C-terminal(s) according to the genetic algorithm of 20.sup.(n−m)/20.sup.n under conditions: n−m=1 or n−m>1 or m=zero, n<infinity, n is not equal to zero, n is an integer, n is the unit of measurement of the length of peptide, one of the 20 EAA is at its C-terminal of each peptide of n-EAA-length-long. For example, if n=5, 3.2 million distinct 5-EAA-length-long peptide sequences of C-terminal orientation could be produced according to algorithm of 20.sup.n. The complete collection of above 3.2 million distinctive 5-EAA-length long peptide sequences has formed a generic penta-expressed-codon-based peptide library/penta-peptide library accordingly. In accordance with Central Dogma, a corresponding generic sense oligonucleotide probe library or a corresponding generic antisense oligonucleotide library could be produced and vice versa. To address a specific problem of gene expression and regulations, the above mentioned libraries could be used alone or and in combination. Therefore, the above mentioned libraries could be integrated or and included into a singular product or and in one method (FIG. 1), (FIG. 2). Therefore, the above mentioned libraries could be integrated or and included into a singular product or and in one method.

### Target Site: 5′-UTR Sites

It is known in the art that 5′-UTR sequence is another preferred targeting area for antisense compounds, particularly antisense morpholino oligonucleotides. The binding to 5′-UTR of mRNA often interfere with progression of ribosomal initiation complex to form 5′-cap. As a result, this hinders the translation of ORF of the targeted mRNA. Generally, 5′-UTR refers to the sequence between the position of 5′-cap structure and the position of a start codon of ORF. The present invention defines 5′-start codon sequence as the common boundary between ORF and 5′-Untranslated Region (5′-UTR). A sequence of 5′-UTR oriented by an initial codon at its 3′-end of a given gene or mRNA of a given length could be identified among the group of linear consecutive DNA or RNA sequences consisting of all possible combinations of 64 codons with an initial codon/start codon at its 3′-end with the same given length. The ordinary level of skill in the pertinent art would recognize that the initial codon/start codon at 3′-end of 5′-UTR is the sequence of orientation. Thus, any and all 3′-end sequences of 5′-UTR with a start codon at its 3′-end of a given length could be produced from its 3′-end starting from the start codon according to the genetic algorithm of 64.sup.(n−m) under conditions: n>m, or n−m=2, or n−m=3, or n−m=4, or n−m=5, or n−m<infinity, neither n nor m is equal to zero, n and m are integers, n is the unit of measurement of the length of 5′-UTR sequence, n represents the entire length of a given 5′-UTR sequence measured by codon, m represents the length of the sequence of orientation which is a selected sequence for the orientation of the entire sequence of 5′-UTR or mRNA which is measured by codon. When n=1 and m=1, position of codon is (m−n)+1. When n−m>1 and n−m<infinity, position of codon is (m−n). The negative sign in front of n indicates that the codon position is at 5′-UTR. For example, if n=3 and m=1, 4,096 distinct 3′-GTA oriented oligonucleotide sequences of three-codon-length-long could be produced according to algorithm of 64.sup.(n−m). The length of three-codon equals nine-nucleotide. The complete collection of above 4,096 distinctive 9-mer oligonucleotide sequences has formed a 9-mer generic sense-codon-based oligonucleotide library accordingly. In accordance with Watson-Crick DNA complementary rule, a corresponding 9-mer generic antisense-codon-based oligonucleotide library could be produced and vice versa. To address a specific problem of gene expression and regulations, the above mentioned libraries could be used alone or and in combination (FIG. 1), (FIG. 2).

### Target Site: 3′-UTR Sites

It is known in the art that 3′-UTR sequence has been shown to have little or no significant homology sequence between members of gene family. Moreover, it does not include common protein domains sequence (Goncalves et al., Strategies 13(3): 93-96, 2000). That trait likely reduces the chance of cross hybridization. Adding to the importance, 3′-UTR often contain several regulatory elements that govern the spatial and temporal expression of an mRNA (Kuersten et al., Nat. Genet. 4: 626-637, 2003). A 20-mer antisense oligonucleotide (PS-ODN, ISIS 5132) directed to 3′-UTR of c-raf mRNA. As a result, the growth of human tumor cell lines had been suppressed obviously (Monia et al., Nat. Med. 2(6): 668-75, 1996). Thus, 3′-UTR is a preferred targeting area for antisense compounds, particularly antisense oligonucleotides as well. In respect of non-canonical genomic events, gene sequencing analysis could be performed by using combinatorial of inventive oligonucleotide libraries for 5′-UTR, ORF and 3′-UTR. The mentioned non-canonical genomic events include but are not limited to genomic deletions, alternative spliced transcriptions, transcripts lacking a 3′ exon and non-polyadenylation. Generally, 3′-UTR refers to the sequence between the position of a stop codon of ORF and the position of Poly (A) tail of 3′-UTR. The present invention defines a 5′-stop codon sequence as the common boundary between ORF and 3′-Untranslated Region (3′-UTR).

A 5′-terminal sequence of 3′-UTR with a stop codon at its 5′-end of a given gene or mRNA of a given length can be identified among the group of linear consecutive DNA or RNA sequences consisting of all possible combinations of 64 codons with a stop codon at its 5′-end with the same length. The stop codon is the sequence of orientation of the mentioned 5′-terminal sequence of 3′-UTR. The ordinary level of skill in the pertinent art would recognize that the stop codon at 5′-terminal of 3′-UTR is the sequence of orientation. Thus, any and all 5′-terminal sequences of 3′-UTR or mRNA with a stop codon at its 5′-end of a given length could be produced from its 5′-end starting from a stop codon according to the genetic algorithm of 64.sup.(n−m) under the conditions: n−m>1, or n−m=2, or n−m=3, or n−m=4, or n−m=5, or n−m<infinity, neither n nor m is equal to zero, both n and m are integers, n is the unit of measurement of the length of 3′-UTR sequence or mRNA, n represents the entire length of a given 3′-UTR sequence or mRNA measured by codon, m represents the length of the sequence of orientation which is a selected sequence for the orientation of the entire 3′-UTR sequence or mRNA which is measured by codon. For example, if n=3 and m=1, one 5′-TGA is at 5′-end, 4,096 distinct 5′-TGA oriented oligonucleotide sequences of three-codon-length-long could be produced according to algorithm of 64.sup.(n−m). The length of three-codon equals nine-nucleotide. The complete collection of above 4,096 distinctive 9-mer oligonucleotide sequences has formed a 9-mer generic codon-based sense oligonucleotide or PCR primer library accordingly. In accordance with Watson-Crick DNA complementary rule, a corresponding 9-mer generic antisense-codon-based antisense oligonucleotide library has been produced and vice versa. To address a specific problem of gene expression and regulations, the above mentioned libraries could be used alone or and in combination (FIG. 2), (FIG. 3). Therefore, the above mentioned libraries could be integrated or and included into a singular product or and in one method.

A 5′-terminal antisense sequence of 3′-UTR with an oligo(T)ₛ sequence at its 5′-end of a given gene or mRNA of a given length can be identified among the group of linear consecutive antisense DNA or RNA sequences consisting of all possible combinations of 64 antisense codons with an oligo(T)ₛ at its 5′-end with the same length. The ordinary level of skill in the pertinent art would recognize that the oligo(T)ₛ at 5′-terminal antisense sequence of 3′-UTR or mRNA is the antisense sequence of orientation. As will be appreciated by one ordinary skilled in the art, when an oligo(T)ₛ has a length of three-antisense-codon-long, s=m=3. When an oligo(T)ₛ has a length of four-antisense-codon-long, s=m=4. When an oligo(T)ₛ has a length of five-antisense-codon-long, s=m=5. When an oligo(T)ₛ has a length of six-antisense-codon-long, s=m=6. When an oligo(T)ₛ has a length of seven-antisense-codon-long, s=m=7. When an oligo(T)ₛ has a length of eight-antisense-codon-long, s=m=8. When an oligo(T)ₛ has a length of nine-antisense-codon-long, s=m=9. When an oligo(T)ₛ has a length of ten-antisense-codon-long, s=m=10. The length of 5′-oligo-d(T)_{S}-3′ could be measured by 5′-TTT. 5′-oligo-d(T)_{S}-3′ is the antisense sequence of orientation. Thus, any and all 5′-end terminal antisense sequences of 3′-UTR or mRNA with an oligo(T)ₛ at its 5′-end of a given length could be produced from its 5′-end of antisense sequence starting from an oligo(T)ₛ according to the antisense genetic algorithm of 64.sup.(n−m) under the conditions: n−m>1, or n−m=2, or n−m=3, or n−m=4, or n−m=5, or n−m<infinity, neither n nor m is equal to zero, both n and m are integers, n is the unit of measurement of the length of antisense sequence of 3′-UTR or mRNA, n represents the entire length of a given antisense sequence of 3′-UTR or mRNA measured by antisense codon, m represents the length of the antisense sequence of orientation which is a selected antisense sequence for the orientation of the entire antisense sequence of 3′-UTR or mRNA which is measured by antisense codon. For example, if n=8, m=6, an oligo(T)ₛ is at 5′-end, 4,096 distinct 5′-oligo(T)ₛ oriented antisense oligonucleotide sequences of eight-antisense-codon-length-long could be produced according to algorithm of 64.sup.(n−m). The length of eight-antisense-codon equals 24-nucleotide. The complete collection of above 4,096 distinctive 24-mer generic antisense oligonucleotide sequences has formed a generic antisense-codon-based antisense oligonucleotide library accordingly. In accordance with Watson-Crick DNA complementary rule, a corresponding 24-mer generic codon-based sense oligonucleotide library has been produced and vice versa. To address a specific problem of gene expression and regulations, the above mentioned libraries could be used alone or and in combination (FIG. 1), (FIG. 2). Therefore, the above mentioned libraries could be integrated or and included into a singular product or and in one method.

### Target Site: Pre-mRNA Splicing Sites

It is known in the art that approximately 50% of disease-related point mutation may results in splicing pattern changes (Lopez-Bigas et al., FEBS Letters 579: 1900-1903, 2005). The relevance between SNPs change and splicing pattern has been reported (Majewski et al., Affymetrix Microarray Bulletin Symposia, 2006). In some cases, more than 60% of genes are known to be alternatively spliced. As a result, hundreds of thousands of transcribed RNA variants with potentially distinct functions were produced (Johnson et al., Science 296: 916-919, 2003). Obviously, pre-mRNA splicing sites are desirable therapeutic targets for antisense compounds. For example, the interfering of morpholino antisense oligonucleotide with pre-mRNA processing steps could prevent snRNP complex from binding to its target at the terminals of Introns (Bruno et al., Hum. Mol. Genet. 3(20): 2409-20, 2004). Generally, Pre-mRNA Splicing Sites refer to 5′-splice donor site and 3′-splice acceptor site in a major splice intron. Many, if not most, the 5′-splice donor site has an almost invariant sequence of GU at 5′-end of the Intron while 3′-splice acceptor site has an almost invariant sequence of AG at 3′-end of the Intron. The present invention defines a codon sequence selected from a group of codons comprising ′5-GUA, 5′-GUC, 5′-GUG and 5′-GUU as the common boundary between 5′-Intron and 3′-Extron. Similarly, the present invention defines a codon sequence selected from a group of codons comprising ′5-AAG, 5′-CAG, 5′-GAG and 5′-UAG as the common boundary between 3′-Intron and 5′-Extron.

A 3′-terminal antisense sequence of 5′-splice donor site with an antisense codon selected from a group of antisense codons comprising 5′-TAC, 5′-GAC, 5′-CAC and 5′-AAC at its 3′-end of a given gene or a given Pre-mRNA of a given length can be identified among the group of linear consecutive DNA or RNA antisense sequences consisting of all possible combinations of 64 antisense codons with an antisense codon selected from a group of antisense codons comprising 5′-TAC, 5′-GAC, 5′-CAC and 5′-AAC at its 3′-end with the same length. Thus, any and all 3′-end terminal antisense sequences of 5′-splice donor site with an antisense codon selected from a group of antisense codons comprising 5′-TAC, 5′-GAC, 5′-CAC and 5′-AAC at its 3′-end of a given length could be produced from its 3′-end including an antisense codon selected from a group of antisense codons comprising 5′-TAC, 5′-GAC, 5′-CAC and 5′-AAC according to the antisense genetic algorithm of 64.sup.(n−m) under the conditions: n−m>1, or n−m=2, or n−m=3, or n−m=4, or n−m=5, or n−m<infinity, neither n nor m is equal to zero, both n and m are integers, n is the unit of measurement of the length of antisense sequence of 5′-splice donor site, n represents the entire length of a given antisense sequence of 5′-splice donor site measured by antisense codon, m represents the length of a selected antisense sequence of terminal orientation for the entire antisense sequence of 5′-splice donor site measured by antisense codon. For example, if n=3 and m=1 (5′-TAC is at 3′-end), 4,096 distinct 5′-TAC oriented antisense oligonucleotide sequences of three-antisense-codon-length long could be produced according to algorithm of 64.sup.(n−m). The length of three-antisense-codon equals nine-nucleotide. The complete collection of above 4,096 distinctive 9-mer antisense oligonucleotide sequences has formed a three-antisense-codon-based antisense oligonucleotide library accordingly. In accordance with Watson-Crick DNA complementary rule, a corresponding 9-mer codon-based sense oligonucleotide library has been produced and vice versa. To address a specific problem of gene expression and regulations, the above mentioned libraries could be used alone or and in combination (FIG. 1), (FIG. 2). Therefore, the above mentioned libraries could be integrated or and included into a singular product or and in one method.

A 3′-terminal antisense sequence of 3′-splice acceptor site with an antisense codon selected from a group of antisense codons comprising 5′-CTT/5′-CUU, 5′-CTG/5′-CUG, 5′-CTC/5′-CUC and 5′-CTA/5′-CUA at its 3′-end of a given gene or a given Pre-mRNA of a given length can be identified among the group of linear consecutive DNA or RNA antisense sequences consisting of all possible combinations of 61 antisense codons with an antisense codon selected from a group of antisense codons comprising 5′-CTT/5′-CUU, 5′-CTG/5′-CUG, 5′-CTC/5′-CUC and 5′-CTA/5′-CUA at its 3′-end with the same length. Thus, any and all 3′-end terminal antisense sequences of 3′-splice acceptor site with an antisense codon selected from a group of antisense codons comprising 5′-CTT/5′-CUU, 5′-CTG/5′-CUG, 5′-CTC/5′-CUC and 5′-CTA/5′-CUA at its 3′-end of a given length could be produced from its 3′-end including an antisense codon selected from a group of antisense codons comprising 5′-CTT/5′-CUU, 5′-CTG/5′-CUG, 5′-CTC/5′-CUC and 5′-CTA/5′-CUA according to the antisense genetic algorithm of 61.sup.(n−m) under the conditions: n−m>1, or n−m=2, or n−m=3, or n−m=4, or n−m=5, or n−m<infinity, neither n nor m is equal to zero, both n and m are integers, n is the unit of measurement of the length of antisense sequence of 3′-splice acceptor site, n represents the entire length of a given antisense sequence of 3′-splice acceptor site measured by antisense codon, m represents the length of a selected antisense sequence of terminal orientation for the entire antisense sequence of 3′-splice acceptor site measured by antisense codon. For example, if n=3 and m=1 (one 5′-CTT of 3′ towards 5′ orientation is at 3′-end), 3,721 distinct 5′-CTT oriented antisense oligonucleotide sequences of three-antisense-codon-length long could be produced according to algorithm of 61.sup.(n−m). The length of three-antisense-codon equals nine-nucleotide. The complete collection of above 3,721 distinctive 9-mer antisense oligonucleotide sequences has formed a 9-mer generic antisense-codon-based antisense oligonucleotide library accordingly. In accordance with Watson-Crick DNA complementary rule, a corresponding 9-mer generic codon-based sense oligonucleotide library has been produced and vice versa. To address a specific problem of gene expression and regulations, the above mentioned libraries could be used alone or and in combination (FIG. 1), (FIG. 2). Therefore, the above mentioned libraries could be integrated or and included into a singular product or and in one method.

### Target Site: Pre-mRNA Alternative Splicing Sites

Pre-mRNA has alternative splicing sites. Those include but are not limited to 5′-UGCAUG (cis-elements) which have been identified as repeated motif downstream of extron EIIIB of fibronectin gene. It has further identified that the two-codon-length long motif is involved cell-type specific alternative pre-mRNA splicing (Huh et al., Genes Dev. 8: 1561-1 1574, 1994), (Lim et al., Mol. Cell Biol. 18:3900-3906, 1998).

A 5′-terminal sequence of Pre-mRNA Alternative Splicing Site with two codons at its 5′-end of a given gene or a given Pre-mRNA of a given length can be identified among the group of linear consecutive DNA or RNA sequences consisting of all possible combinations of 64 codons with two codons at its 5′-end with the same length. The ordinary level of skill in the pertinent art would recognize that the two codons at 5′-terminal of Pre-mRNA Alternative Splicing Site is the sequence of orientation. Thus, any and all 5′-terminal sequences of Pre-mRNA Alternative Splicing Site with two codons at its 5′-end of a given length could be produced from its 5′-end including two codons according to the genetic algorithm of 64.sup.(n-m) under the conditions: n−m>1, or n−m=2, or n−m=3, or n−m=4, or n−m=5, or n−m<infinity, neither n nor m is equal to zero, both n and m are integers, n is the unit of measurement of the length of Pre-mRNA Alternative Splicing Site, n represents the entire length of a given Pre-mRNA Alternative Splicing Site sequence measured by codon, m represents the length of a selected sequence of terminal orientation for the entire Pre-mRNA Alternative Splicing Site sequence measured by codon. For example, if n=4 and m=2 (one 5′-UGCAUG is at 5′-end), 4,096 distinct 5′-UGCAUG oriented oligonucleotide sequences of four-codon-length long could be produced according to algorithm of 64.sup.(n−m). The length of four-codon equals twelve-nucleotide. The complete collection of above 4,096 distinctive 12-mer oligonucleotide sequences has formed a 12-mer generic codon-based oligonucleotide probe or PCR primer library accordingly. In accordance with Watson-Crick DNA complementary rule, a corresponding 12-mer generic antisense-codon-based antisense oligonucleotide library has been produced and vice versa. To address a specific problem of gene expression and regulations, the above mentioned libraries could be used alone or and in combination (FIG. 1), (FIG. 2). Therefore, the above mentioned libraries could be integrated or and included into a singular product or and in one method.

### Target Site: Micro RNAs' Sites

Micro RNAs (miRNA) is typically 21-mer to 23-mer non-coding RNA transcribed from genomic DNA. Generally, miRNA regulates the expression of other genes instead of being translated into a peptide in a similar manner as RNA interference (RNAi). Presumably, there are more than 10% of genes in human genome contain a target site for miRNA (John et al., PLoS Biol 2(11): e363, 2004). The targeting site could be exemplified by a targeting site in Ribozymes. Ribozymes are RNA molecules that function as enzyme in a similar manner of RNA interference (RNAi). Ribozymes occur naturally in vivo, but could be engineered in vitro for RNA interference of specific sequences. Hairpin ribozymes cleave the target RNA immediately upstream sequences of 5′-GUC. Hammerhead ribozymes cleave the target RNA at codon selected from a group of codons comprising 5′-AUA, 5′-AUU, 5′-AUC, 5′-UUA, 5′-UUU, 5′-UUC, 5′-GUA, 5′-GUU, 5′-GUC, 5′-CUA, 5′-CUU and 5′-CUC.

A 5′-terminal sequence of Hammerhead Ribozymes Cleave Site (Micro RNAs Site) with a codon selected from a group of codons comprising 5′-AUA, 5′-AUU, 5′-AUC, 5′-UUA, 5′-UUU, 5′-UUC, 5′-GUA, 5′-GUU, 5′-GUC, 5′-CUA, 5′-CUU and 5′-CUC can be identified among the group of linear consecutive DNA or RNA sequences consisting of all possible combinations of 64 codons with a codon selected from a group of codons comprising 5′-AUA, 5′-AUU, 5′-AUC, 5′-UUA, 5′-UUU, 5′-UUC, 5′-GUA, 5′-GUU, 5′-GUC, 5′-CUA, 5′-CUU and 5′-CUC at its 5′-end with the same length. The ordinary level of skill in the pertinent art would recognize that a codon selected from a group of codons comprising 5′-AUA, 5′-AUU, 5′-AUC, 5′-UUA, 5′-UUU, 5′-UUC, 5′-GUA, 5′-GUU, 5′-GUC, 5′-CUA, 5′-CUU and 5′-CUC at 5′-terminal of Hammerhead Ribozymes Cleave Site is the sequence of orientation. Thus, any and all 5′-terminal sequences of Hammerhead Ribozymes Cleave Site with a codon selected from a group of codons comprising 5′-AUA, 5′-AUU, 5′-AUC, 5′-UUA, 5′-UUU, 5′-UUC, 5′-GUA, 5′-GUU, 5′-GUC, 5′-CUA, 5′-CUU and 5′-CUC at its 5′-end of a given length could be produced from its 5′-end including a codon selected from a group of codons comprising 5′-AUA, 5′-AUU, 5′-AUC, 5′-UUA, 5′-UUU, 5′-UUC, 5′-GUA, 5′-GUU, 5′-GUC, 5′-CUA, 5′-CUU and 5′-CUC according to the genetic algorithm of 64.sup.(n−m) under the conditions: n−m>1, or n−m=2, or n-m=3, or n−m=4, or n−m=5, or n−m<infinity, neither n nor m is equal to zero, both n and m are integers, n is the unit of measurement of the length of Hammerhead Ribozymes Cleave Site, n represents the entire length of a given Hammerhead Ribozymes Cleave Site sequence measured by codon, m represents the length of a selected sequence of terminal orientation for the entire Hammerhead Ribozymes Cleave Site sequence measured by codon. For example, if n=3 and m=1 (5′-AUA is at 5′-end), 4,096 distinct 5′-AUA oriented oligonucleotide sequences of three-codon-length long could be produced according to algorithm of 64.sup.(n−m). The length of three-codon equals nine-nucleotide. The complete collection of above 4,096 distinctive 9-mer oligonucleotide sequences has formed a 9-mer generic codon-based oligonucleotide probe or PCR primer library accordingly. In accordance with Watson-Crick DNA complementary rule, a corresponding 9-mer generic antisense-codon-based antisense oligonucleotide library has been produced and vice versa. To address a specific problem of gene expression and regulations, the above mentioned libraries could be used alone or and in combination (FIG. 1), (FIG. 2). Therefore, the above mentioned libraries could be integrated or and included into a singular product or and in one method.

### Target Site: Mutations and SNPs Sites

The point mutations, deletions, insertion and single nucleotide polymorphisms (SNPs) may occur in coding regions or non-coding regions such as 5′-UTR and 3′-UTR. SNPs are the most frequent type of genetic variation in the genome. SNPs are highly conserved throughout evolution. Moreover, it is highly conserved within a population. There are approximately over 10 million SNPs that have been identified in human genome (Sherry et al., Nucleic Acids Res. 29: 308-311, 2001). Therefore, the map of SNPs could provide a unique genotypic marker or genetic signature for a specified population or even for an individual. In terms of functionality, those genetic variations including SNPs occurred in coding regions are actually a change(s) of codon(s) or and ORF(s). For example, 5′-GCA encodes Alanine. If G, the single nucleotide of the first position of 5′-GCA, is swapped for an alternate (C, A and T), 5′-CCA encodes Proline; 5′-ACA encodes Threonine; 5′-TCA encodes Serine. If C, the single nucleotide of the second position of 5′-GCA, is swapped for an alternate (G, A and T), 5′-GGA encodes Glycine; 5′-GAA encodes Glutamic acid; 5′-GTA encodes Valine. If A, the single nucleotide of the third position of 5′-GCA, is swapped for an alternate (G, C and T), 5′-GCG encodes Alanine; 5′-GCC encodes Alanine; 5′-GCT encodes Alanine. 5′-GGA encodes Glycine. If G, the single nucleotide of the first position of 5′-GGA, is swapped for T, 5′-GGA will become 5′-TGA, terminator of the peptide chain. 5′-TAA, 5′-TGA and 5′-TAG encode peptide termination respectively. The substitution of any nucleotide at any position of the triplet codons of the three terminators will turn the terminator into a codon for a specific amino acid or another terminator. For example, If T, the single nucleotide of the first position of 5′-TGA, is swapped for an alternate (G, C and A), 5′-TGA, terminator of the peptide chain will become 5′-GGA, 5′-CGA and 5′-AGA which encodes Glycine, Arginine and Arginine respectively. If G, the single nucleotide of the second position of 5′-TGA, is swapped for an alternate (T, C and A), 5′-TGA, terminator of the peptide chain will become 5′-TTA, 5′-TCA and 5′-TAA which encodes Leucine, Serine and termination respectively. If A, the single nucleotide of the third position of 5′-TGA, is swapped for an alternate (G, C and T), 5′-TGA, terminator of the peptide chain will become 5′-TGG, 5′-TGC and 5′-TGT which encodes Tryptophan, Cysteine and Cysteine respectively. The substitution, replacement, deletion and insertion of single or multiple nucleotide(s) in the coding region could cause the shift of ORF(s) and the change(s) of codon(s), the termination of peptide chain and/or the merger of two or more peptide chains together. In appearance, the point mutation, deletion, insertion and SNPs in the coding region is a change(s) of nucleotide(s). In nature, it is actually a change(s) of codon(s) or and ORF(s). Therefore, codon-based methods could address the nature of those phenomena more directly in comparison with the nucleotide-based methods. It is known in the art that a high density nucleotide-based SNP array is type of DNA microarrays platforms, which is specialized in detection of SNPs or Loss Of Heterozygosity (LOH). The ordinary level of skill in the pertinent art would recognize that generic codon-based oligonucleotide library has genuine genome-wide scope of a given length for SNPs' screening and identifying. The mentioned generic codon-based oligonucleotide libraries include SNPs detection automatically and make ready for creating a high density codon-based SNP array as probe libraries. This is superior to nucleotide-based SNP array since it has systematically eliminated all redundant oligonucleotides existed in nucleotide-based SNP array for targeting coding regions. The ordinary level of skill in the pertinent art would also recognize that in accordance with Watson-Crick DNA complementary rule, a corresponding generic genome-wide antisense-codon-based antisense oligonucleotide library for SNPs' screening and identifying could be produced and vice versa. To address a specific problem of gene expression and regulations, the above mentioned libraries could be used alone or and in combination (FIG. 1), (FIG. 2). Therefore, the above mentioned libraries could be integrated or and included into a singular product or and in one method.

### Target Site: Codon Substitution and Exception Sites

The genetic code has been evolving. Exceptions and changes exist. For example, 5′-TGA, which usually codes for the termination of the synthesis of a peptide chain, sometimes codes for selenocysteine, an amino acid which is not among the 20 essential amino acids. Other exceptions such as 5′-AGA and 5′-ATA are not usable in Micrococcus luteus while 5′-CGG is not usable in Mycoplasmas and Spiroplasmas (Kanoi et al., J. Mol. Bio. 230: 51-56, 1993), (Oba et al., Proc. Natl. Acad. Sci. U.S.A. 88: 921-925, 1991). Both 5′-TAA and 5′-TAG encode Glutamine in Tetrahymena, Paramecium and Acetabularia of Cilliates and Algae while 5′-CTG encodes Serine in Candida cylindrica of Fungi (Tourancheau et al., EMBO J. 14: 3262-3267, 1995). However, all above genetic algorithms are applicable to those exceptions as long as the corresponding codon(s) are substituted accordingly. Examples of codon(s) substitution include but are by no means limited to mammalian mitochondria such as human mitochondria. Examples of codon(s) substitution include but are by no means limited to start codon substitution such as the substitution of 5′-ATG from 5′-ATA of mammalian mitochondria. Therefore, a specific genome-wide single stranded codon-based oligonucleotide probe or PCR primer library such as mammalian mitochondria oligonucleotide library could be established from a generic genome-wide oligonucleotide library according to a specifically defined set of codons or and specially defined codon substitution(s). Since the present invention allows targeting site, such as a codon site to be substituted by any one of 61 amino acid coding codons for ORFs or 64 codons for 5′-UTRs and 3′-UTRs, a given site of ORF or 5′-UTR or 3′-UTR and their corresponding downstream or upstream sequences could be targeted or produced specifically by the inventive probes. One ordinary skilled in the relevant art would recognize that in accordance with Watson-Crick DNA complementary rule, a corresponding genome-wide single stranded antisense-codon-based antisense oligonucleotide library could be produced and vice versa. One ordinary skilled in the relevant art would also recognize that in accordance with Central Dogma, a corresponding genome-wide expressed-codon-based peptide library could be produced either directly from mentioned sense oligonucleotide probe library or indirectly from its corresponding antisense oligonucleotide library and vice versa. To address a specific problem of gene expression and regulations, the above mentioned libraries could be used alone or and in combination (FIG. 1), (FIG. 2). Therefore, the above mentioned libraries could be integrated or and included into a singular product or and in one method.

### Targeting Probes

Generally, there are two major types of probes for genome-wide DNA and RNA detecting. One is cDNA probe. Another one is oligonucleotide probe which include sense oligonucleotide and antisense oligonucleotide.

Traditionally, cDNAs were often chosen as probes for genome-wide gene screening such as DNA microarrays. However, the maintenance and replication of a genome-wide cDNA library demands quality controls. It can be time-consuming and add to the cost of production (Knight et al., Nature 414: 135-136, 2001). A cDNA library is a specialized library that may even have cell type specifics. Such characteristics set a limit for its applications. Another drawback is the probability of contamination during production. Zacharewski's laboratory has sequenced 1,189 cDNAs of a set of probes of DNA microarrays. Only 62% of them definitely represent the correct sequences (Halgren et al., Nucleic Acids Res. 29: 582-588, 2001). Up to 30% error rates of cDNA probes were also identified by three major centers of DNA microarrays (Knight, Nature 410: 860-861, 2001). When a singular full-length cDNA probe was employed to detect a single target in a nucleic acids sample, it often demonstrates a specific and reproducible hybridization result under optimized experimental conditions. Whereas, when massive full-length cDNA molecules were employed as genome-wide probe on DNA microarrays, it often surprisingly brings out cross hybridization results due to the homology domains among gene family members and non-gene family members. Moreover, up to date, there is no method of isolating massive sense strands from massive antisense strands and vice versa at systematical and global level. Therefore, it seems as if it is no way to figure out which cDNA strand is exactly viewed on the DNA microarrays through the hybridization signal detection system. Clearly, there is still a need of inventing new probe libraries, which have genuine genome-wide screening spectrum with more accuracy but low cost. Chemically synthesized oligonucleotides provide an alternative option. The process of chemical synthesis prevents problems from possible bacterial contamination and preserves the accuracy of designed sequences. Short oligonucleotides (9-30 mers) could be used as primer in Polymerase Chain Reaction (hereinafter PCR) whereas cDNA molecules generally could not be used as PCR primers. Moreover, systematically synthesized antisense oligonucleotides could be used to construct a genome-wide antisense array to target sense strands at global level. Similarly, its counterpart could be used to construct a genome-wide sense array to screen, identify and validate antisense leads at genome-wide spectrum and vice versa. Notably, chemical modifications could enable antisense oligonucleotide to obtain higher affinity to its targeting sequence without probe length elongation, increasing resistance to nucleases within a cell and more effective penetration of cellular membranes. Antisense oligonucleotides make up the major component of antisense drugs, which currently have over 20 antisense drugs in clinical trials to treat various diseases. More than half of those trials are now in Phase II or later stage clinical development.

### Sufficient Length of Oligonucleotide

The probability study of priming site in DNA with 45,000 base pair indicated that P(0), the probability of no priming site of 12-mer oligonucleotides, is 0.995. P(1), the probability of exactly one priming site of 12-mer oligonucleotides, is 0.005. P(>1), the probability of more than one priming site of 12-mer oligonucleotides, is <10⁻⁴(<10.sup.−4) (Studier, Proc. Natl. Acad. Sci. U.S.A. 86: 6917-6921, 1989). It is known in the art that oligonucleotides ranging from 6 mers to 24 mers in length are sufficient as probes in hybridization. An oligonucleotide as short as a 6 mers could perform reliable hybridization and efficient priming (Drmanac et al., DNA and Cell Biology 9: 527-534, 1990), (Feinberg et al., Anal. Biochem. 132: 6-13, 1983). On solid surface, 6-mer oligonucleotide arrays have been reported (Timofeev et al., Nucleic Acids Res. 29(12): 2626-2634, 2001). 9-mer oligonucleotide arrays have been utilized in DNA fingerprinting (Reyes-Lopez et al., Nucleic Acids Res. 31(2): 779-789, 2003). 9-mer oligonucleotides tethered to glass were capable of capturing their complementary DNA strands as long as 1,300 bases in length with good discrimination against mismatches in hybridization (Beattie et al., Mol. Biotechnol. 4: 213-225, 1995). A mutation scan of a second of 1.2 kb HIV variant sample containing 27 single base substitutions had been performed on an 8-mer and 9-mer oligonucleotide arrays respectively. 96.3% of the mutations were detected on the 8-mer oligonucleotide array while 100% of the mutations were detected on the 9-mer array (Gunderson et. al., genome Res. 8: 1142-1153, 1998). Single-base mismatch detection by 12-mer oligonucleotide probes were demonstrated on electrostatic readout of DNA microarrays (Clack et al., Nat. Biotech. 26(7): 825-830, 2008). In aqueous phase, 9-mer oligonucleotide has been performed as a PCR primer (Williams et al., Nucleic Acids Res. 18: 6531-6535, 1990). Research has further revealed that the incorporation of Locked Nucleic Acid hereinafter LNA to short oligonucleotides could increase their thermal stabilities towards complementary DNA and RNA in PCR and hybridization (Babu et al., Nucleic Acids Res. 22: 1317-1319, 2003). In antisense area, 13-mer antisense oligonucleotides complementary to Rous Sarcoma Virus mRNA were shown to inhibit virus replication (Zamecnic et. al., Proc. Natl. Acad. Sci. U.S.A. 75(1): 280-284, 1978). One of the major concerns of antisense oligonucleotides is the specificity of the modulations to the flow of genetic information. Conceptually, Longer the length is, more specific the probe will be. However, long oligonucleotides (>10 mers) may decrease the specificity if its binding affinity is high (Herschlag et al., Proc. Natl. Acad. Sci. U.S.A. 88: 6921-6925, 1991). In practice, 12-25 nucleotide-long antisense oligonucleotides were frequently employed in experiments (Woolf et al., Proc. Natl. Acad. Sci. USA 89: 7305-7309, 1992). The specificity of inhibition of short antisense oligonucleotides (7-8 nucleotide-long with C-5 propyne primidines and phosphorothioate internucleotide linkages) has also been explored (Wagner et al., Nat. Biotechnol. 14: 840-844, 1996). The disadvantage of using short oligonucleotide is the frequency of non-specific binding. The advantage is the higher capacity of discriminating mismatches than longer probes in hybridization (Drmanac et al., DNA and Cell Biology 9: 527-534, 1990). Milner et al. speculated that longer oligonucleotides might have internal base pairing which prevent duplex formation, or that duplex formation was inhibited by dangling ends of single stranded oligonucleotides that could not fit into the folded structure of mRNA (Milner et al., Nat. Biotechnol. 15: 537-541, 1997). For dsRNA oligonucleotide, a short one is desirable since longer one may cause interferon response in RNA interference (Paddison et al., Proc. Natl. Acad. Sci. USA 99(3): 1443-1448, 2002). Considering the increasing probability of forming secondary structure(s) that accompanies the increasing length of an oligonucleotide; a short oligonucleotide has certain advantages over a longer one though longer ones in general are more specific. Short oligonucleotides are also relatively inexpensive and suitable for large-scale production.

### Rational Design of Sense and Antisense Oligonucleotide

Up to date, none of the current genome sequence data such as Human, Saccharomyces cerevisiae, Caenorhabditis elegans, Drosophila melanogaster and Escherichia coli include all genetic divergence such as mutations, insertions and Single Nucleotide Polymorphisms (SNPs). Even none of the current SNPs sequence databases has a complete set of genuine genome-wide SNPs sequences. A set or library of arbitrarily (randomly) selected oligonucleotides either as primers or probes are unlikely to be unbiased (T. Chen et. al., U.S. patent application Ser. No. 10/869,055, 2004). It poses a great challenge for many areas in life science and medicine, such as antisense pharmaceutical lead discovery and validation. Obviously, it is desirable to have a genuine generic oligonucleotide system which has a full-range genome screening spectrum for all cells, tissues, organs and organisms in forms of sense and antisense. Traditionally, the generic oligonucleotide library was constructed by all possible combinations of A.T.G.C. according to algorithm of 4.sup.n (Studier, Proc. Natl. Acad. Sci. U.S.A. 86: 6917-6921, 1989), (Szybalski, Gene 90: 177-178, 1990). In algorithm of 4.sup.n, n denotes length measurement unit of oligonucleotide; n represents nucleotide. Designing DNA arrays according with algorithm of 4.sup.n was proposed in the art (Barinaga, Science 253:1489, 1991). According to algorithm of 4.sup.n, Affymetrix Inc. has actually developed commercialized nucleotide-based generic oligonucleotide arrays (Lipshutz et al., Nat. Genet. 21: 20-24, 1999). Universal n-mer arrays, constructed based on algorithm of 4.sup.n, were also proposed (Michael van Dam et al., Genome Research 12:145-152, 2001). Though oligonucleotide microarrays are widely applied but poorly understood (Pozhitkov et al., Briefings in Functional Genomics and Proteomics, 2007). One ordinary skilled in the relevant art would recognize that though an oligonucleotide array could correspond to either sense or antisense strand, a single array should be made entirely of sense or antisense strands. It is crucial to know whether sense or antisense strands have been viewed on the array for subsequent analysis. Identifying two different strands from each other is a pre-requisite for studying. Moreover, oligonucleotide set or library constructed by all possible combinations of four nucleotides cannot discriminate target sequences among non-coding, coding and regulatory regions at massive scale. Second, even within a targeting coding region, template strand (antisense) and non-template strand (sense) would be targeted indiscriminately by those nucleotide-based generic oligonucleotides in hybridization. Third, one of the analytical areas of gene functionality is in coding regions, but the algorithm of 4.sup.n is not a codon-based approach. Fourth, the algorithm of 4.sup.n inevitably includes huge amount of non-sense codons that virtually do not exist in ORF. The redundancy is phenomenal and hinders the accuracy of hybridization. It increases the cost of production and complicates the operation. For example, for 24-mer oligonucleotides, the number of all possible combinations of 61 codons according to algorithm of 61.sup.(n−m) is 382,742,836,021 [61.sup.(8-1)], wherein n=8 and m=1. Whereas, the number of all possible combinations of four nucleotides according to algorithm of 4.sup.n is 281,474,976,710,656 (4.sup.24), wherein n=24. In accordance with Watson-Crick DNA complementary rule, a corresponding counterpart of 24-mer antisense-codon-based antisense sense oligonucleotide library has been produced and vice versa. For 24-mer antisense oligonucleotides, the number of all possible combinations of 61 antisense codons according to algorithm of 61.sup.(n−m) is 382,742,836,021 [61.sup.(8-1)], wherein n=8 and m=1. Whereas, the number of all possible combinations of four nucleotides according to algorithm of 4.sup.n is 281,474,976,710,656 (4.sup.24), wherein n=24. Remarkably, the redundancy is 89.6 times more than the virtual ORF sequences (Table 1). Furthermore, the enormous redundant sequences are wrong probe sequences and should be eliminated completely when targeting ORF regions. In the manufacture's point of view, producing a 24-mer antisense oligonucleotide library for generic antisense oligonucleotide array by present invention is 89.6 times more cost-effective than the traditional design based on algorithm of 4.sup.n (Table 1). That efficiency will increase further with the elongation of the length of the antisense oligonucleotide following algorithm of 4.sup.3.times.n divided by 61.sup.(n−m) (Table 1). The redundancy of antisense oligonucleotide sequence with specified length could be calculated in accordance with the algorithm of 4³ⁿ−61^{(n-m)} (Table 1). Fifth, since nucleotide-based generic oligonucleotide array was constructed according to algorithm of 4.sup.n, the GC contents among the oligonucleotides vary from 0% to 100%. Once thousands of oligonucleotides with variable GC content are immobilized on one piece of solid support, all of them will be exposed to a unique hybridization environment. Thus, a considerable number of the oligonucleotide probes may have to hybridize under un-optimized conditions. Consequently, false positive or negative hybridization results might be produced. Applying 2.4 to 3.0 M tetramethyl ammonium or tetraethyl ammonium chloride (Wood et al., Proc. Natl. Acad. Sci. U.S.A. 82: 1585-1588, 1985) as buffer (Fodor et al., U.S. Pat. No. 6,197,506) may reduce some effects of the GC bias in hybridization to a certain degree. However, the effect of such reagents has its limitations.

To address the above issues, the present invention proposes a series of codon-based and antisense-codon-based generic oligonucleotide libraries constructed according to a series of corresponding inventive algorithms. From the structure point of view, the inventive codon-based and antisense-codon-based generic oligonucleotides automatically include all possible point mutations, SNPs, and exogenous genetic factors within the designed probe sequences of a given length at genome-wide scope. From the function point of view, on genome scope, codon-based generic oligonucleotides could systematically differentiate targeting antisense strands from sense strands and vice versa. From the technology point of view, as will be appreciated by ordinary skilled in the art, sequence of orientation of oligonucleotide, is one of the major innovative features of present invention, which orients the entire codon-based or and antisense-codon-based sequences as probes or and primers systematically in the operation. Thus, the sequence of orientation has automatically standardized the codon-based oligonucleotides in a specified library. The antisense sequence of orientation has automatically standardized the antisense-codon-based antisense oligonucleotides in a specified library. From the methodology point of view, single stranded codon-based oligonucleotide, single stranded antisense-codon-based oligonucleotide and single stranded expression-codon-based peptide libraries are all correlated in design. They represent three major dimensions of an integrated inventive platform as a Systems Biology approach. From the manufacture point of view, a combinatorial codon-based generic library contains distinctive oligonucleotides that are large enough to afford genome-wide screening for all life forms, yet small enough for fabrication and readout. The universality, convertibility and standardization are the major features of the products. Taking separately or together, the inventive codon-based design is superior in many respects to traditional design based on nucleotides.

It has the capacity of targeting all possible endogenous and exogenous genes simultaneously for a given nucleic acid sample related to a biological or pathological or medical process or pathway. It is characterized by its unique all-purpose generic usage, regardless of genetic variations among cell types, tissues, organs, individuals and species. Moreover, codon-based oligonucleotide has a unique structure of the sequence of orientation. For a non-limiting example, a start codon could be used as the sequence of orientation. A start codon (5′-ATG) oriented codon-based oligonucleotides could target specific sequences in a sample of nucleic acids. They could be used as a library of upstream primers for PCR. With oligo-d(T)ₛ as downstream primer, a corresponding cDNA library could be subsequently obtained from a given mRNA sample aided by RT-PCR. The cDNA library could then be used as probe library for cDNA Arrays. The protocols of making and using cDNA Arrays are known in the art. The current invention presents sense oligonucleotide probes, which were designed according to template strand of cDNA under DNA complementarity's rules (FIG. 2) and vice versa. It presents antisense oligonucleotide probes, which were designed according to non-template strand of cDNA under DNA complementarity's rules (FIG. 2) and vice versa. Hence, a brief review of gene structures (FIG. 1) for the probe design would be helpful.

### SUMMARY OF THE INVENTION

### Genome-Wide Antisense Oligonucleotide Libraries

There is also provided a DNA oligonucleotide library comprising a plurality of DNA oligonucleotides, wherein each of the oligonucleotides is represented by the formula 5′-(C_{S}).sub.n-3′, wherein C_{S} represents an amino acid coding codon, wherein n is an integer, wherein n represents the length of said sense oligonucleotide measured by codon.

There is also provided a RNA oligonucleotide library comprising a plurality of RNA oligonucleotides, wherein each of the oligonucleotides is represented by the formula 5′-(C_{S}).sub.n-3′, wherein C_{S} represents an amino acid coding codon, wherein n is an integer, wherein n represents the length of said sense oligonucleotide measured by codon.

There is also provided an antisense DNA oligonucleotide library comprising a plurality of antisense DNA oligonucleotides, wherein each of antisense oligonucleotides, in accordance with Watson-Crick DNA complementary rule, is an responding oligonucleotide represented by the formula 5′-(C_{S}).sub.n-3′, wherein C_{S} represents an amino acid coding codon, wherein n is an integer, wherein n represents the length of said sense oligonucleotide measured by codon.

There is also provided an antisense RNA oligonucleotide library comprising a plurality of antisense RNA oligonucleotides, wherein each of antisense oligonucleotides, in accordance with Watson-Crick DNA complementary rule, is an responding oligonucleotide represented by the formula 5′-(C_{S}).sub.n-3′, wherein C_{S} represents an amino acid coding codon, wherein n is an integer, wherein n represents the length of said sense oligonucleotide measured by codon.

There is also provided an antisense DNA oligonucleotide library comprising a plurality of antisense DNA oligonucleotides, wherein each of the antisense oligonucleotides is represented by the formula 5′-(C_{A}).sub.n-3′, wherein C_{A} represents an antisense amino acid coding codon, wherein n is an integer, wherein n represents the length of said antisense oligonucleotide measured by antisense codon.

There is also provided an antisense RNA oligonucleotide library comprising a plurality of antisense RNA oligonucleotides, wherein each of the antisense oligonucleotides is represented by the formula 5′-(C_{A}).sub.n-3′, wherein C_{A} represents an antisense amino acid coding codon, wherein n is an integer, wherein n represents the length of said antisense oligonucleotide measured by antisense codon.

There is also provided a DNA oligonucleotide library comprising a plurality of DNA oligonucleotides, wherein each of the oligonucleotides is represented by the formula 5′-(C_{S}).sub.m(C_{S}).sub.n-3′, wherein C_{S} represents an amino acid coding codon, wherein n is an integer, wherein n>1, wherein n<10, wherein n represents the length of said sense oligonucleotide measured by codon, m is an integer, wherein m<n, wherein m<7, wherein m represents the length of sequence of orientation measured by codon.

There is also provided a RNA oligonucleotide library comprising a plurality of RNA oligonucleotides, wherein each of the oligonucleotides is represented by the formula 5′-(C_{S}).sub.m(C_{S}).sub.n-3′, wherein C_{S} represents an amino acid coding codon, wherein n is an integer, wherein n>1, wherein n<10, wherein n represents the length of said sense oligonucleotide measured by codon, m is an integer, wherein m<n, wherein m<7, wherein m represents the length of sequence of orientation measured by codon.

There is also provided an antisense DNA oligonucleotide library comprising a plurality of antisense DNA oligonucleotides, wherein each of antisense oligonucleotides, in accordance with Watson-Crick DNA complementary rule, is an responding oligonucleotide represented by the formula 5′-(C_{S}).sub.m(C_{S}).sub.n-3′, wherein C_{S} represents an amino acid coding codon, wherein n is an integer, wherein n>1, wherein n<10, wherein n represents the length of said sense oligonucleotide measured by codon, wherein m is an integer, wherein m<n, wherein m<7, wherein m represents the length of sequence of orientation measured by codon.

There is also provided an antisense RNA oligonucleotide library comprising a plurality of antisense RNA oligonucleotides, wherein each of antisense oligonucleotides, in accordance with Watson-Crick DNA complementary rule, is an responding oligonucleotide represented by the formula 5′-(C_{S}).sub.m(C_{S}).sub.n-3′, wherein C_{S} represents an amino acid coding codon, wherein n is an integer, wherein n>1, wherein n<10, wherein n represents the length of said sense oligonucleotide measured by codon, wherein m is an integer, wherein m<n, wherein m<7, wherein m represents the length of sequence of orientation measured by codon.

There is also provided an antisense DNA oligonucleotide library comprising a plurality of antisense DNA oligonucleotides, wherein each of the antisense oligonucleotides is represented by the formula 5′-(C_{A}).sub.m(C_{A}).sub.n-3′, wherein C_{A} represents an antisense amino acid coding codon, wherein n is an integer, wherein n>1, wherein n<10, wherein n represents the length of said antisense oligonucleotide measured by antisense codon, wherein m is an integer, wherein m<n, wherein m<7, wherein m represents the length of antisense sequence of orientation measured by antisense codon.

There is also provided an antisense RNA oligonucleotide library comprising a plurality of antisense RNA oligonucleotides, wherein each of the antisense oligonucleotides is represented by the formula 5′-(C_{A}).sub.m(C_{A}).sub.n-3′, wherein C_{A} represents an antisense amino acid coding codon, wherein n is an integer, wherein n>1, wherein n<10, wherein n represents the length of said antisense oligonucleotide measured by antisense codon, wherein m is an integer, wherein m<n, wherein m<7, wherein m represents the length of antisense sequence of orientation measured by antisense codon.

There is also provided a DNA oligonucleotide library comprising a plurality of DNA oligonucleotides, wherein each of the oligonucleotides is represented by the formula 5′-(V_{S}).sub.n-3′, wherein V_{S} represents a codon, wherein n is an integer, wherein n represents the length of said sense oligonucleotide measured by codon.

There is also provided a RNA oligonucleotide library comprising a plurality of RNA oligonucleotides, wherein each of the oligonucleotides is represented by the formula 5′-(V_{S}).sub.n-3′, wherein V_{S} represents a codon, wherein n is an integer, wherein n represents the length of said sense oligonucleotide measured by codon.

There is also provided an antisense DNA oligonucleotide library comprising a plurality of antisense DNA oligonucleotides, wherein each of antisense oligonucleotides, in accordance with Watson-Crick DNA complementary rule, is an responding oligonucleotide represented by the formula 5′-(V_{S}).sub.n-3′, wherein V_{S} represents a codon, wherein n is an integer, wherein n represents the length of said sense oligonucleotide measured by codon.

There is also provided an antisense RNA oligonucleotide library comprising a plurality of antisense RNA oligonucleotides, wherein each of antisense oligonucleotides, in accordance with Watson-Crick DNA complementary rule, is an responding oligonucleotide represented by the formula 5′-(V_{S}).sub.n-3′, wherein V_{S} represents a codon, wherein n is an integer, wherein n represents the length of said sense oligonucleotide measured by codon.

There is also provided an antisense DNA oligonucleotide library comprising a plurality of antisense DNA oligonucleotides, wherein each of the antisense oligonucleotides is represented by the formula 5′-(V_{A}).sub.n-3′, wherein V_{A} represents an antisense codon, wherein n is an integer, wherein n represents the length of said antisense oligonucleotide measured by antisense codon.

There is also provided an antisense RNA oligonucleotide library comprising a plurality of antisense RNA oligonucleotides, wherein each of the antisense oligonucleotides is represented by the formula 5′-(V_{A}).sub.n-3′, wherein V_{A} represents an antisense codon, wherein n is an integer, wherein n represents the length of said antisense oligonucleotide measured by antisense codon.

There is also provided a DNA oligonucleotide library comprising a plurality of DNA oligonucleotides, wherein each of the oligonucleotides is represented by the formula 5′-(V_{S}).sub.m(V_{S}).sub.n-3′, wherein C_{S} represents a codon, wherein n is an integer, wherein n>1, wherein n<10, wherein n represents the length of said sense oligonucleotide measured by codon, wherein m is an integer, wherein m<n, wherein m<7, wherein m represents the length of sequence of orientation measured by codon.

There is also provided a RNA oligonucleotide library comprising a plurality of RNA oligonucleotides, wherein each of the oligonucleotides is represented by the formula 5′-(V_{S}).sub.m(V_{S}).sub.n-3′, wherein C_{S} represents a codon, wherein n is an integer, wherein n>1, wherein n<10, wherein n represents the length of said sense oligonucleotide measured by codon, wherein m is an integer, wherein m<n, wherein m<7, wherein m represents the length of sequence of orientation measured by codon.

There is also provided an antisense DNA oligonucleotide library comprising a plurality of antisense DNA oligonucleotides, wherein each of antisense oligonucleotides, in accordance with Watson-Crick DNA complementary rule, is an responding oligonucleotide represented by the formula 5′-(V_{S}).sub.m(V_{S}).sub.n-3′, wherein C_{S} represents a codon, wherein n is an integer, wherein n>1, wherein n<10, wherein n represents the length of said sense oligonucleotide measured by codon, wherein m is an integer, wherein m<n, wherein m<7, wherein m represents the length of sequence of orientation measured by codon.

There is also provided an antisense RNA oligonucleotide library comprising a plurality of antisense RNA oligonucleotides, wherein each of antisense oligonucleotides, in accordance with Watson-Crick DNA complementary rule, is an responding oligonucleotide represented by the formula 5′-(V_{S}).sub.m(V_{S}).sub.n-3′, wherein C_{S} represents a codon, wherein n is an integer, wherein n>1, wherein n<10, wherein n represents the length of said sense oligonucleotide measured by codon, wherein m is an integer, wherein m<n, wherein m<7, wherein m represents the length of sequence of orientation measured by codon.

There is also provided an antisense DNA oligonucleotide library comprising a plurality of antisense DNA oligonucleotides, wherein each of the antisense oligonucleotides is represented by the formula 5′-(V_{A}).sub.m(V_{A}).sub.n-3′, wherein V_{A} represents an antisense codon, wherein n is an integer, wherein n>1, wherein n<10, wherein n represents the length of said antisense oligonucleotide measured by antisense codon, wherein m is an integer, wherein m<n, wherein m<7, wherein m represents the length of antisense sequence of orientation measured by antisense codon.

There is also provided an antisense RNA oligonucleotide library comprising a plurality of antisense RNA oligonucleotides, wherein each of the antisense oligonucleotides is represented by the formula 5′-(V_{A}).sub.m(V_{A}).sub.n-3′, wherein V_{A} represents an antisense codon, wherein n is an integer, wherein n>1, wherein n<10, wherein n represents the length of said antisense oligonucleotide measured by antisense codon, wherein m is an integer, wherein m<n, wherein m<7, wherein m represents the length of antisense sequence of orientation measured by antisense codon.

There is also provided a DNA oligonucleotide library comprising a plurality of DNA oligonucleotides, wherein each of said oligonucleotides is represented by said formula 5′-(V_{S}).sub.n-3′, wherein V_{S} represents a codon, wherein n is an integer, wherein n represents the length of the said oligonucleotide measured by codon(s), wherein each said oligonucleotide further comprising a linker at either 5′-end or 3′-end of said oligonucleotide, wherein the said linker being selected from the group consisting of: sense termination codons; antisense termination codons; sense codons; two consecutive sense codons; two consecutive sense codons of restriction endonuclease recognition site; two consecutive antisense codons of antisense restriction endonuclease recognition site; three consecutive sense codons; a consecutive oligo-d(T)ₛ consisting of a plurality of thymidine nucleotides; a sense codon comprising one universal base; a sense codon comprising two universal bases; a sense codon comprising three universal bases; a sense codon comprising one Locked Nucleic Acid; a sense codon comprising two Locked Nucleic Acids; a sense codon comprising three Locked Nucleic Acids and combinations thereof.

There is also provided a RNA oligonucleotide library comprising a plurality of RNA oligonucleotides, wherein each of said oligonucleotides is represented by said formula 5′-(V_{S}).sub.n-3′, wherein V_{S} represents a codon, wherein n is an integer, wherein n represents the length of the said oligonucleotide measured by codon(s), wherein each said oligonucleotide further comprising a linker at either 5′-end or 3′-end of said oligonucleotide, wherein the said linker being selected from the group consisting of: sense termination codons; antisense termination codons; sense codons; two consecutive sense codons; two consecutive sense codons of restriction endonuclease recognition site; two consecutive antisense codons of antisense restriction endonuclease recognition site; three consecutive sense codons; a consecutive oligo-d(T)ₛ consisting of a plurality of thymidine nucleotides; a sense codon comprising one universal base; a sense codon comprising two universal bases; a sense codon comprising three universal bases; a sense codon comprising one Locked Nucleic Acid; a sense codon comprising two Locked Nucleic Acids; a sense codon comprising three Locked Nucleic Acids and combinations thereof.

According to a terminology aspect of the invention, wherein I_{S} represents sense initiation codon, wherein T_{S} represents sense termination codon, wherein C_{S} represents sense amino acid coding codon, wherein V_{S} represents a sense codon, wherein R_{S} represents two sense codons (six nucleotides) of restriction endonuclease recognition site with the proviso that neither of the two codons is a termination codon, wherein E_{S} represents a two sense codons (six nucleotides) of restriction endonuclease recognition site, wherein oligo-d(T)_{S} represents a plurality of consecutive thymidine nucleotides, wherein I_{A} represents antisense initiation codon, wherein T_{A} represents antisense termination codon, wherein C_{A} represents antisense amino acid coding codon, wherein V_{A} represents antisense codon, wherein R_{A} represents a two antisense codons (six nucleotides) of antisense restriction endonuclease recognition site with the proviso that neither of the two antisense codons is an antisense termination codon, wherein E_{A} represents a two antisense codons (six nucleotides) of antisense restriction endonuclease recognition site, wherein A represents an amino acid, wherein M represents an amino acid encoded by an initiation codon, wherein R_{E} is one of the amino acid sequences encoded by R_{S}, wherein said universal bases are selected from a group comprising 5′-nitroindole-2′-deoxyriboside, 3-nitropyrrole, inosine, pypoxanthine and combinations thereof.

According to method aspect of the invention, there is a method(s) provided for identifying targeting sequences within a sample comprising at least one of the following:
(1) A method of generating a genome-wide sense oligonucleotide library comprising a plurality of sense-codon-based oligonucleotides, wherein oligonucleotide library has a complexity according to an algorithm, wherein said algorithm is 61^{(n-m)}, wherein 61 represents the number of amino acid coding codons, wherein each of said oligonucleotides is represented by a structural formula 5′-(O_{S})ₘ (C_{S})ₙ-3′, wherein O_{S} is a sequence of orientation having a length of m codons and C_{S} is an amino acid coding codon, wherein n is the number of codons, wherein said oligonucleotides comprise a sequence of orientation located at 5′-end, wherein said sequence of orientation consists of a selected sequence having m codons in length, wherein said m represents the length of said sequence of orientation measured by codon, wherein n is an integer, wherein n>zero, wherein n=24 or n<24, wherein m is an integer, wherein m>zero, wherein n>m, wherein (n−m) represents n minus m, wherein n−m>1, wherein n−m<9, wherein (n−m) represents the entire length of said oligonucleotide, wherein 61^{(n-m)} represents the number of oligonucleotide in said library, wherein according to Watson-Crick DNA complementary rule, a corresponding antisense-codon-based antisense oligonucleotides have been produced and formed a library of antisense oligonucleotide.
(2) A method of generating an antisense oligonucleotide library comprising a plurality of antisense oligonucleotides, wherein said antisense oligonucleotide library has a complexity according to an algorithm, wherein said algorithm is 61^{(n-m)}, wherein 61 represents the number of antisense amino acid coding codons, wherein the length of said antisense oligonucleotides has (n−m) antisense—codon-length long, wherein said n represents the length of said antisense oligonucleotides measured by antisense codon, wherein said antisense oligonucleotides have antisense sequence of orientation, wherein the said antisense sequence of orientation consists of a selected antisense sequence, wherein the length of said antisense sequence of orientation has m-antisense-codon-length long, wherein said m represents the length of said antisense sequence of orientation measured by antisense codon, wherein n is an integer, wherein n>zero, wherein m is an integer, wherein m>zero, wherein n>m, wherein (n−m) represents n minus m, wherein n−m<9, wherein (n−m) represents the entire length of said antisense oligonucleotide, wherein 61^{(n-m)} represents the number of antisense oligonucleotide in said library.
(3) A method of generating an antisense mammalian mitochondria oligonucleotide library comprising a plurality of antisense oligonucleotides, wherein said antisense oligonucleotide library has a complexity according to an algorithm, wherein said algorithm is 60^{(n-m)}, wherein 60 represents the number of antisense mammalian mitochondria codons, wherein the length of said antisense oligonucleotides has (n−m) antisense—codon-length long, wherein said n represents the length of said antisense oligonucleotides measured by antisense codon, wherein said antisense oligonucleotides have antisense sequence of orientation, wherein the said antisense sequence of orientation consists of a selected antisense sequence, wherein the length of said antisense sequence of orientation has m-antisense-codon-length long, wherein said m represents the length of said antisense sequence of orientation measured by antisense codon, wherein n is an integer, wherein n>zero, wherein m is an integer, wherein m>zero, wherein n>m, wherein (n−m) represents n minus m, wherein n−m<9, wherein (n−m) represents the entire length of said antisense oligonucleotide, wherein 60^{(n-m)} represents the number of antisense mammalian mitochondria oligonucleotide in said library.
(4) A method of generating an antisense oligonucleotide library according to method 1 or 2 or 3, wherein each said antisense oligonucleotide further comprises a linker at either 5′-end or 3′-end of said antisense oligonucleotides; wherein said linker being selected from a group consisting antisense sense initiation codons; antisense termination codon; antisense amino acid coding codon; two consecutive antisense codons consisting an antisense restriction enzyme site; and combinations thereof.
(5) A method of generating an antisense oligonucleotide library according to method 1 or 2 or 3 or 4, wherein n−m=2, wherein said oligonucleotides are grouped according to GC content, wherein said GC content are selected from a group consisting of 16.67% GC content, 33.33% GC content, 50.00% GC content, 66.67% GC content, 83.33% GC content and 100.00% GC content (Table 2).
(6) A method of generating an antisense oligonucleotide library according to method 1 or 2 or 3 or 4, wherein n−m=3, wherein said oligonucleotides are grouped according to GC content, wherein said GC content are selected from a group consisting of 11.11% GC content, 22.22% GC content, 33.33% GC content, 44.44% GC content, 55.56% GC content, 66.67% GC content, 77.78% GC content, 88.89 GC content and 100.00% GC content (Table 2).
(7) A method of generating an antisense oligonucleotide library according to method 1 or 2 or 3 or 4, wherein n−m=4, wherein said oligonucleotides are grouped according to GC content, wherein said GC content are selected from a group consisting of 8.33% GC content, 16.67% GC content, 25.00% GC content, 33.33% GC content, 41.67% GC content, 50.00% GC content, 58.33% GC content, 66.67% GC content, 75.00% GC content, 83.33 GC content, 91.67% GC content and 100.00% GC content (Table 2).
(8) A method of generating an antisense oligonucleotide library according to method 1 or 2 or 3 or 4, wherein n−m=5, wherein said oligonucleotides are grouped according to GC content, wherein said GC content are selected from a group consisting of 6.67% GC content, 13.33% GC content, 20.00% GC content, 26.67% GC content, 33.33% GC content, 40.00% GC content, 46.67% GC content, 53.33% GC content, 60.00% GC content, 66.67% GC content, 73.33% GC content, 80.00% GC content, 86.67 GC content, 93.33% GC content and 100.00% GC content (Table 2).
(9) A method of generating an antisense oligonucleotide library according to method 1 or 2 or 3 or 4, wherein n−m=6, wherein said oligonucleotides are grouped according to GC content, wherein said GC content are selected from a group consisting of 5.56% GC content, 11.11% GC content, 16.67% GC content, 22.22% GC content, 27.78% GC content, 33.33% GC content, 38.89% GC content, 44.44% GC content, 50.00% GC content, 55.56% GC content, 61.11% GC content, 66.67% GC content, 72.22% GC content, 77.78% GC content, 83.33% GC content, 88.89 GC content, 94.44% GC content and 100.00% GC content (Table 2).
(10) A method of generating an antisense oligonucleotide library according to method 1 or 2 or 3 or 4, wherein n−m=7, wherein said oligonucleotides are grouped according to GC content, wherein said GC content are selected from a group consisting of 4.76% GC content, 9.52% GC content, 14.29% GC content, 19.05% GC content, 23.81% GC content, 28.57% GC content, 33.33% GC content, 38.10% GC content, 42.86% GC content, 47.62% GC content, 52.38% GC content, 57.14% GC content, 61.90% GC content, 66.67% GC content, 71.43% GC content, 76.19% GC content, 80.95% GC content, 85.71 GC content, 90.48% GC content, 95.24% GC content and 100.00% GC content (Table 2).
(11) A method of generating an antisense oligonucleotide library according to method 1 or 2 or 3 or 4, wherein n−m=8, wherein said oligonucleotides are grouped according to GC content, wherein said GC content are selected from a group consisting of 4.12% GC content, 8.33% GC content, 12.50% GC content, 16.67% GC content, 20.83% GC content, 25.00% GC content, 29.17% GC content, 33.33% GC content, 37.50% GC content, 41.67% GC content, 45.83% GC content, 50.00% GC content, 54.17% GC content, 58.33% GC content, 62.50% GC content, 66.67% GC content, 70.83% GC content, 75.00% GC content, 79.17% GC content, 83.33% GC content, 87.50% GC content, 91.67% GC content, 95.83% GC content and 100% GC content (Table 2).

According to product aspect of the invention, there is a kit(s) provided for identifying targeting sequences within a sample comprising at least one of the following:

a 5′ start codon (sense) panel comprising a plurality of oligonucleotides, wherein each of said oligonucleotides is represented by the formula 5′-Oₘ(C_{S})ₙ-3′, wherein n1 represents the length of said (C_{S})ₙ₁ measured by codon, n1 is variable and an integer;

a 5′ start codon (antisense) panel comprising a plurality of oligonucleotides, wherein each of the oligonucleotides is represented by the formula 5′-(C_{A})ₙ₂I_{A}-3′, wherein n2 represents the length of said (C_{A})ₙ₂ measured by codon, n2 is variable and an integer;

a 5′ UTR (sense) panel comprising a plurality of oligonucleotides, wherein each of the oligonucleotides is represented by the formula 5′-(V_{S})ₙ₃I_{S}-3′, wherein n3 represents the length of said (V_{S})ₙ₃ measured by codon, n3 is variable and an integer;

a 5′ UTR (antisense) panel comprising a plurality of oligonucleotides, wherein each of the oligonucleotides is represented by the formula 5′-I_{A}(V_{A})ₙ₄-3′, wherein n4 represents the length of said (V_{A})ₙ₄ measured by codon, n4 is variable and an integer;

a 3′ stop codon (sense) panel comprising a plurality of oligonucleotides, wherein each of the oligonucleotides is represented by the formula 5′-(C_{S})ₙ₅T_{S}-3′, wherein n5 represents the length of said (C_{S})ₙ₅ measured by codon, n5 is variable and an integer;

a 3′ stop codon (antisense) panel comprising a plurality of oligonucleotides, wherein each of the oligonucleotides is represented by the formula 5′-T_{A}(C_{A})ₙ₆-3′, wherein n6 represents the length of said (C_{A})ₙ₆ measured by codon, n6 is variable and an integer;

a 3′ UTR (sense) panel comprising a plurality of oligonucleotides, wherein each of the oligonucleotides is represented by the formula 5′-T_{S}(V_{S})ₙ₇-3′, wherein n7 represents the length of said (V_{S})ₙ₇ measured by codon, n7 is variable and an integer;

a 3′ UTR (antisense) panel comprising a plurality of oligonucleotides, wherein each of the oligonucleotides is represented by the formula 5′-(V_{A})ₙ₈T_{A}-3′, wherein n8 represents the length of said (V_{A})ₙ₈ measured by codon, n8 is variable and an integer;

a 5′ restriction endonuclease (sense) panel comprising a plurality of oligonucleotides, wherein each of the oligonucleotides is represented by the formula 5′-R_{S}(C_{S})ₙ₉-3′, wherein n9 represents the length of said (C_{S})ₙ₉ measured by codon, n9 is variable and an integer;

a 5′ restriction endonuclease (antisense) panel comprising a plurality of oligonucleotides, wherein each of the oligonucleotides is represented by the formula 5′-(C_{A})ₙ₁₀R_{A}-3′, wherein n10 represents the length of said (C_{S})ₙ₁₀ measured by codon, n10 is variable and an integer;

a 3′ restriction endonuclease (sense) panel comprising a plurality of oligonucleotides, wherein each of the oligonucleotides is represented by the formula 5′-(C_{S})ₙ₁₁R_{S}-3′, wherein n11 represents the length of said (C_{S})ₙ₁₁ measured by codon, n11 is variable and an integer;

a 3′ restriction endonuclease (antisense) panel comprising a plurality of oligonucleotides, wherein each of the oligonucleotides is represented by the formula 5′-R_{A}(C_{A})ₙ₁₂-3′, wherein n12 represents the length of said (C_{A})ₙ₁₂ measured by codon, n12 is variable and an integer;

a 5′ restriction endonuclease (sense) panel comprising a plurality of oligonucleotides, wherein each of the oligonucleotides is represented by the formula 5′-E_{S}(V_{S})ₙ₁₃-3′, wherein n13 represents the length of said (V_{S})ₙ₁₃ measured by codon, n13 is variable and an integer;

a 5′ restriction endonuclease (antisense) panel comprising a plurality of oligonucleotides, wherein each of the oligonucleotides is represented by the formula 5′-(V_{A})ₙ₁₄E_{A}-3′, wherein n14 represents the length of said (V_{A})ₙ₁₄ measured by codon, n14 is variable and an integer;

a 3′ restriction endonuclease (sense) panel comprising a plurality of oligonucleotides, wherein each of the oligonucleotides is represented by the formula 5′-(V_{S})ₙ₁₅E_{S}-3′, wherein n15 represents the length of said (V_{S})ₙ₁₅ measured by codon, n15 is variable and an integer;

a 3′ restriction endonuclease (antisense) panel comprising a plurality of oligonucleotides, wherein each of the oligonucleotides is represented by the formula 5′-E_{A}(V_{A})ₙ₁₆-3′, wherein n16 represents the length of said (V_{A})ₙ₁₆ measured by codon, n16 is variable and an integer;

a between 5′ and 3′ (sense) panel comprising a plurality of oligonucleotides, wherein each of the oligonucleotides is represented by the formula 5′-(C_{S})ₙ₁₇-3′, wherein n17 represents the length of said (C_{S})ₙ₁₇ measured by codon, n17 is variable and an integer;

a between 5′ and 3′ (antisense) panel comprising a plurality of oligonucleotides, wherein each of the oligonucleotides is represented by the formula 5′-(C_{A})ₙ₁₈-3′, wherein n18 represents the length of said (C_{A})ₙ₁₈ measured by codon, n18 is variable and an integer;

an oligonucleotide panel comprising a plurality of oligonucleotides, wherein each of the said oligonucleotides is represented by the formula 5′-(C_{S})ₙ₁₉-3′, wherein each said oligonucleotide further comprises a linker at either 5′-end or 3′-end of said oligonucleotide, the said linker is an amino acid coding codon in sense orientation, n19 represents the length of said (C_{S})ₙ₁₉ measured by codon, n19 is variable and an integer;

an oligonucleotide panel comprising a plurality of oligonucleotides, wherein each of the said oligonucleotides is represented by the formula 5′-(C_{S})ₙ₂₀-3′, wherein each said oligonucleotide further comprises a linker at either 5′-end or 3′-end of said oligonucleotide, the said linker is two consecutive amino acid coding codons in sense orientation, n20 represents the length of said (C_{S})ₙ₂₀ measured by codon, n20 is variable and an integer;

an oligonucleotide panel comprising a plurality of oligonucleotides, wherein each of the said oligonucleotides is represented by the formula 5′-(C_{S})ₙ₂₁-3′, each said oligonucleotide further comprises a linker at either 5′-end or 3′-end of said oligonucleotide, the said linker is three consecutive amino acid coding codons in sense orientation, n21 represents the length of said (C_{S})ₙ₂₁ measured by codon, n21 is variable and an integer;

an oligonucleotide panel comprising a plurality of oligonucleotides, wherein each of the said oligonucleotides is represented by the formula 5′-(C_{S})ₙ₂₂-3′, each said oligonucleotide further comprises a linker at either 5′-end or 3′-end of said oligonucleotide, the said linker is a codon comprising one universal base, n22 represents the length of said (C_{S})ₙ₂₂ measured by codon, n22 is variable and an integer;

an oligonucleotide panel comprising a plurality of oligonucleotides, wherein each of the said oligonucleotides is represented by the formula 5′-(C_{S})ₙ₂₃-3′, wherein each said oligonucleotide further comprises a linker at either 5′-end or 3′-end of said oligonucleotide, the said linker is a codon comprising two universal bases, n23 represents the length of said (C_{S})ₙ₂₃ measured by codon, n23 is variable and an integer;

an oligonucleotide panel comprising a plurality of oligonucleotides, wherein each of the said oligonucleotides is represented by the formula 5′-(C_{S})ₙ₂₄-3′, wherein each said oligonucleotide further comprises a linker at either 5′-end or 3′-end of said oligonucleotide, the said linker is a codon comprising three universal bases, n24 represents the length of said (C_{S})ₙ₂₄ measured by codon, n24 is variable and an integer;

an oligonucleotide panel comprising a plurality of oligonucleotides, wherein each of the said oligonucleotides is represented by the formula 5′-(C_{A})ₙ₂₅-3′, wherein each said oligonucleotide further comprises a linker at either 5′-end or 3′-end of said oligonucleotide, the said linker is an amino acid coding codon in antisense orientation, n25 represents the length of said (C_{A})ₙ₂₅ measured by codon, n25 is variable and an integer;

an oligonucleotide panel comprising a plurality of oligonucleotides, wherein each of the said oligonucleotides is represented by the formula 5′-(C_{A})ₙ₂₆-3′, wherein each said oligonucleotide further comprises a linker at either 5′-end or 3′-end of said oligonucleotide, the said linker is two consecutive amino acid coding codons in antisense orientation, n26 represents the length of said (C_{A})ₙ₂₆ measured by codon, n26 is variable and an integer;

an oligonucleotide panel comprising a plurality of oligonucleotides, wherein each of the said oligonucleotides is represented by the formula 5′-(C_{A})ₙ₂₇-3′, wherein each said oligonucleotide further comprises a linker at either 5′-end or 3′-end of said oligonucleotide, the said linker is three consecutive amino acid coding codons in antisense orientation, n27 represents the length of said (C_{A})ₙ₂₇ measured by codon, n27 is variable and an integer;

an oligonucleotide panel comprising a plurality of oligonucleotides, wherein each of the said oligonucleotides is represented by the formula 5′-(C_{A})ₙ₂₈-3′, wherein each said oligonucleotide further comprises a linker at either 5′-end or 3′-end of said oligonucleotide, the said linker is a codon comprising one universal base, n28 represents the length of said (C_{A})ₙ₂₈ measured by codon, n28 is variable and an integer;

an oligonucleotide panel comprising a plurality of oligonucleotides, wherein each of the said oligonucleotides is represented by the formula 5′-(C_{A})ₙ₂₉-3′, wherein each said oligonucleotide further comprises a linker at either 5′-end or 3′-end of said oligonucleotide, the said linker is a codon comprising two universal bases, n29 represents the length of said (C_{A})ₙ₂₉ measured by codon, n29 is variable and an integer;

an oligonucleotide panel comprising a plurality of oligonucleotides, wherein each of the said oligonucleotides is represented by the formula 5′-(C_{A})ₙ₃₀-3′, wherein each said oligonucleotide further comprises a linker at either 5′-end or 3′-end of said oligonucleotide, the said linker is a codon comprising three universal bases, n30 represents the length of said (C_{A})ₙ₃₀ measured by codon, n30 is variable and an integer;

an oligonucleotide panel comprising a plurality of oligonucleotides, wherein each of the said oligonucleotides is represented by said formula 5′-(V_{S})ₙ₃₁-3′, wherein each said oligonucleotide further comprising a linker at either 5′-end or 3′-end of said oligonucleotide, the said linker is a codon in sense orientation, n31 represents the length of said (V_{S})ₙ₃₁ measured by codon, n31 is variable and an integer;

an oligonucleotide panel comprising a plurality of oligonucleotides, wherein each of the said oligonucleotides is represented by said formula 5′-(V_{S})ₙ₃₂-3′, wherein each said oligonucleotide further comprising a linker at either 5′-end or 3′-end of said oligonucleotide, the said linker is two consecutive codons in sense orientation, n32 represents the length of said (V_{S})ₙ₃₂ measured by codon, n32 is variable and an integer;

an oligonucleotide panel comprising a plurality of oligonucleotides, wherein each of the said oligonucleotides is represented by said formula 5′-(V_{S})ₙ₃₃-3′, wherein each said oligonucleotide further comprising a linker at either 5′-end or 3′-end of said oligonucleotide, the said linker three consecutive codons in sense orientation, n33 represents the length of said (V_{S})ₙ₃₃ measured by codon, n33 is variable and an integer;

an oligonucleotide panel comprising a plurality of oligonucleotides, wherein each of the said oligonucleotides is represented by said formula 5′-(V_{S})ₙ₃₄-3′, wherein each said oligonucleotide further comprising a linker at either 5′-end or 3′-end of said oligonucleotide, the said linker is a codon comprising one universal base, n34 represents the length of said (V_{S})ₙ₃₄ measured by codon, n34 is variable and an integer;

an oligonucleotide panel comprising a plurality of oligonucleotides, wherein each of the said oligonucleotides is represented by said formula 5′-(V_{S})ₙ₃₅-3′, wherein each said oligonucleotide further comprising a linker at either 5′-end or 3′-end of said oligonucleotide, the said linker is a codon comprising two universal bases, n35 represents the length of said (V_{S})ₙ₃₅ measured by codon, n35 is variable and an integer;

an oligonucleotide panel comprising a plurality of oligonucleotides, wherein each of the said oligonucleotides is represented by said formula 5′-(V_{S})ₙ₃₆-3′, wherein each said oligonucleotide further comprising a linker at either 5′-end or 3′-end of said oligonucleotide, the said linker is a codon comprising three universal bases, n36 represents the length of said (V_{S})ₙ₃₆ measured by codon, n36 is variable and an integer;

an oligonucleotide panel comprising a plurality of oligonucleotides, wherein each of the said oligonucleotides is represented by said formula 5′-(V_{S})ₙ₃₇-3′, wherein each said oligonucleotide further comprising a linker at either 5′-end or 3′-end of said oligonucleotide, the said linker is a codon comprising one LNA, n37 represents the length of said (V_{S})ₙ₃₇ measured by codon, n37 is variable and an integer;

an oligonucleotide panel comprising a plurality of oligonucleotides, wherein each of the said oligonucleotides is represented by said formula 5′-(V_{S})ₙ₃₈-3′, wherein each said oligonucleotide further comprising a linker at either 5′-end or 3′-end of said oligonucleotide, the said linker is a codon comprising two LNAs, n38 represents the length of said (V_{S})ₙ₃₈ measured by codon, n38 is variable and an integer;

an oligonucleotide panel comprising a plurality of oligonucleotides, wherein each of the said oligonucleotides is represented by said formula 5′-(V_{S})ₙ₃₉-3′, wherein each said oligonucleotide further comprising a linker at either 5′-end or 3′-end of said oligonucleotide, the said linker is a codon comprising three LNAs, n39 represents the length of said (V_{S})ₙ₃₉ measured by codon, n39 is variable and an integer;

an oligonucleotide panel comprising a plurality of oligonucleotides, wherein each of the said oligonucleotides is represented by said formula 5′-(V_{S})ₙ₄₀-3′, wherein each said oligonucleotide further comprising a linker at either 5′-end or 3′-end of said oligonucleotide, the said linker is a codon comprising one MF, n40 represents the length of said (V_{S})ₙ₄₀ measured by codon, n40 is variable and an integer;

an oligonucleotide panel comprising a plurality of oligonucleotides, wherein each of the said oligonucleotides is represented by said formula 5′-(V_{S})ₙ₄₁-3′, wherein each said oligonucleotide further comprising a linker at either 5′-end or 3′-end of said oligonucleotide, the said linker is a codon comprising two MFs, n41 represents the length of said (V_{S})ₙ₄₁ measured by codon, n41 is variable and an integer;

an oligonucleotide panel comprising a plurality of oligonucleotides, wherein each of the said oligonucleotides is represented by said formula 5′-(V_{S})ₙ₄₂-3′, wherein each said oligonucleotide further comprising a linker at either 5′-end or 3′-end of said oligonucleotide, the said linker is a codon comprising three MFs, n42 represents the length of said (V_{S})ₙ₄₂ measured by codon, n42 is variable and an integer;

an oligonucleotide panel comprising a plurality of oligonucleotides, wherein each of the said oligonucleotides is represented by said formula 5′-(V_{S})ₙ₄₃-3′, wherein each said oligonucleotide further comprising a linker at either 5′-end or 3′-end of said oligonucleotide, the said linker is a codon comprising one PNA, n43 represents the length of said (V_{S})ₙ₄₃ measured by codon, n43 is variable and an integer;

an oligonucleotide panel comprising a plurality of oligonucleotides, wherein each of the said oligonucleotides is represented by said formula 5′-(V_{S})ₙ₄₄3′, wherein each said oligonucleotide further comprising a linker at either 5′-end or 3′-end of said oligonucleotide, the said linker is a codon comprising two PNAs, n44 represents the length of said (V_{S})ₙ₄₄ measured by codon, n44 is variable and an integer;

an oligonucleotide panel comprising a plurality of oligonucleotides, wherein each of the said oligonucleotides is represented by said formula 5′-(V_{S})ₙ₄₅-3′, wherein each said oligonucleotide further comprising a linker at either 5′-end or 3′-end of said oligonucleotide, the said linker is a codon comprising three PNAs, n45 represents the length of said (V_{S})ₙ₄₅ measured by codon, n45 is variable and an integer;

an oligonucleotide panel comprising a plurality of oligonucleotides, wherein each of the said oligonucleotides is represented by said formula 5′-(V_{S})ₙ₄₆-3′, wherein each said oligonucleotide further comprising a linker at either 5′-end or 3′-end of said oligonucleotide, the said linker is a codon comprising one 2′-MOE, n46 represents the length of said (V_{S})ₙ₄₆ measured by codon, n46 is variable and an integer;

an oligonucleotide panel comprising a plurality of oligonucleotides, wherein each of the said oligonucleotides is represented by said formula 5′-(V_{S})ₙ₄₇-3′, wherein each said oligonucleotide further comprising a linker at either 5′-end or 3′-end of said oligonucleotide, the said linker is a codon comprising two 2′-MOEs, n47 represents the length of said (V_{S})ₙ₄₇ measured by codon, n47 is variable and an integer;

an oligonucleotide panel comprising a plurality of oligonucleotides, wherein each of the said oligonucleotides is represented by said formula 5′-(V_{S})ₙ₄₈-3′, wherein each said oligonucleotide further comprising a linker at either 5′-end or 3′-end of said oligonucleotide, the said linker is a codon comprising three 2′-MOEs, n48 represents the length of said (V_{S})ₙ₄₈ measured by codon, n48 is variable and an integer;

an oligonucleotide panel comprising a plurality of oligonucleotides, wherein each of the said oligonucleotides is represented by said formula 5′-(V_{S})ₙ₄₉-3′, wherein each said oligonucleotide further comprising a linker at either 5′-end or 3′-end of said oligonucleotide, the said linker is a codon comprising one PS, n49 represents the length of said (V_{S})ₙ₄₉ measured by codon, n49 is variable and an integer;

an oligonucleotide panel comprising a plurality of oligonucleotides, wherein each of the said oligonucleotides is represented by said formula 5′-(V_{S})ₙ₅₀-3′, wherein each said oligonucleotide further comprising a linker at either 5′-end or 3′-end of said oligonucleotide, the said linker is a codon comprising two PSs, n50 represents the length of said (V_{S})ₙ₅₀ measured by codon, n50 is variable and an integer;

an oligonucleotide panel comprising a plurality of oligonucleotides, wherein each of the said oligonucleotides is represented by said formula 5′-(V_{S})ₙ₅₁-3′, wherein each said oligonucleotide further comprising a linker at either 5′-end or 3′-end of said oligonucleotide, the said linker is a codon comprising three PSs, n51 represents the length of said (V_{S})ₙ₅₁ measured by codon, n51 is variable and an integer;

an oligonucleotide panel comprising a plurality of oligonucleotides, wherein each of the said oligonucleotides is represented by said formula 5′-(V_{S})ₙ₅₂oligo-d(T)_{S}-3′, wherein the length of said d(T)_{S} is measured by nucleotide, the said s is variable and an integer, the value of the said s is from 21 to 6, wherein n52 represents the length of said (V_{S})ₙ₅₂ measured by codon, n52 is variable and an integer;

an oligonucleotide panel comprising a plurality of oligonucleotides, wherein each of the said oligonucleotides is represented by said formula 5′-oligo-d(T)_{S}-3′, wherein the length of said d(T)_{S} is measured by nucleotide, the said s is variable and an integer, the value of the said s is from 21 to 6; and combinations thereof.

According to each said formula, each of said oligonucleotide panels comprise substantially all of said oligonucleotides.

According to each said formula, each of said oligonucleotide panels consist essentially of said oligonucleotides.

According to one said formula, each of the said oligonucleotides of entire length is organized into different sets, each said sets has at least two identical oligonucleotides, the said sets are further organized into different GC identical panels within the specific selections of GC content; wherein each said oligonucleotides of entire length is represented by n, the said n is a variable and integer, the said n represents n1+1, n2+1, n3+1, n4+1, n5+1, n6+1, n7+1, n8+1, n9+2, n10+2, n11+2, n12+2, n13+2, n14+2, n15+2, n16, n17, n18, n19+1, n20+2, n21+3, n22+1, n23+1, n24+1, n25+1, n26+2, n27+3, n28+1, n29+1, n30+1, n31+1 n32+2, n33+3, n34+1, n35+1, n36+1, and n37+s respectively; wherein the said specific selections of GC content are 0%, 16.67%, 33.33%, 50%, 66.67%, 83.33% and 100% when n equals two; wherein the said specific selections of GC content are 0%, 11.11%, 22.22%, 33.33%, 44.44%, 55.56%, 66.67%, 77.78%, 88.89% and 100% when n equals three; wherein the said specific selections of GC content are 0%, 8.33%, 16.67%, 25%, 33.33%, 41.67%, 50%, 58.33%, 66.67%, 75%, 83.33%, 91.67% and 100% when n equals four; wherein the said specific selections of GC content are 0%, 6.67%, 13.33%, 20%, 26.67%, 33.33%, 40%, 46.67%, 53.33%, 60%, 66.67%, 73.33%, 80%, 86.67%, 93.33% and 100% when n equals five; wherein the said specific selections of GC content are 0%, 5.56%, 11.11%, 16.67%, 22.22%, 27.78%, 33.33%, 38.89%, 44.44%, 50%, 55.56%, 61.11%, 66.67%, 72.22%, 77.78%, 83.33%, 88.89%, 94.44% and 100% when n equals six; wherein the said specific selections of GC content are 0%, 4.76%, 9.52%, 14.29%, 19.05%, 23.81%, 28.57%, 33.33%, 38.10%, 42.86%, 47.62%, 52.38%, 57.14%, 61.90%, 66.67%, 71.43%, 76.19%, 80.95%, 85.71%, 90.48%, 95.24% and 100% when n equals seven, wherein the said specific selections of GC content are 0%, 4.17%, 8.33%, 12.50%, 16.67%, 20.83%, 25%, 29.17%, 33.33%, 37.50%, 41.67%, 45.53%, 50%, 54.17%, 58.33%, 62.50%, 66.67%, 70.83%, 75%, 79.17%, 83.33%, 87.50%, 91.67%, 95.83% and 100% when n equals eight;

each of the said oligonucleotide GC identical panel, wherein each of the said oligonucleotides is represented by a formula selected from a group of formulae described above, wherein each of the said oligonucleotides is immobilized or linked or associate or attached or integrated to a carrier for delivery such as Lentiviruses, Adenoviruses, lipidoids, amphoteric liposomes, nanoparticles such as chitosan nanoparticles and other suitable carriers for antisense oligonucleotide delivery known in the art. In a set of each said oligonucleotide, the said set comprising at least two copies of the said oligonucleotide. The said oligonucleotide comprises at least two said sets. As will be appreciated by one of skilled in the art, the panels may be used alone or in combination. According to each said formula, each of said oligonucleotide panels comprise substantially all of said oligonucleotides. According to each said formula, each of said oligonucleotide panels consist essentially of said oligonucleotides.

According to an application aspect of the invention, there is a kit(s) provided PCR oligonucleotide primer(s) for identifying and amplifying targeting sequences within a sample comprising at least one oligonucleotide selected from the group consisting of:

an oligonucleotide represented by the formula 5′-I_{S}(C_{S})ₙ₁-3′, wherein n1 represents the length of said (C_{S})ₙ₁ measured by codon, n1 is variable and an integer;

an oligonucleotide represented by the formula 5′-(C_{A})ₙ₂I_{A}-3′, wherein n2 represents the length of said (C_{A})ₙ₂ measured by codon, n2 is variable and an integer;

an oligonucleotide represented by the formula 5′-(V_{S})ₙ₃I_{S}-3′, wherein n3 represents the length of said (V_{S})ₙ₃ measured by codon, n3 is variable and an integer;

an oligonucleotide represented by the formula 5′-I_{A}(V_{A})ₙ₄-3′, wherein n4 represents the length of said (V_{A})ₙ₄ measured by codon, n4 is variable and an integer;

an oligonucleotide represented by the formula 5′-(C_{S})ₙ₅T_{S}-3′, wherein n5 represents the length of said (C_{S})ₙ₅ measured by codon, n5 is variable and an integer;

an oligonucleotide represented by the formula 5′-T_{A}(C_{A})ₙ₆-3′, wherein n6 represents the length of said (C_{A})ₙ₆ measured by codon, n6 is variable and an integer;

an oligonucleotide represented by the formula 5′-T_{S}(V_{S})ₙ₇-3′, wherein n7 represents the length of said (V_{S})ₙ₇ measured by codon, n7 is variable and an integer;

an oligonucleotide represented by the formula 5′-(V_{A})ₙ₈T_{A}-3′, wherein n8 represents the length of said (V_{A})ₙ₈ measured by codon, n8 is variable and an integer;

an oligonucleotide represented by the formula 5′-R_{S}(C_{S})ₙ₉-3′, wherein n9 represents the length of said (C_{S})ₙ₉ measured by codon, n9 is variable and an integer;

an oligonucleotide represented by the formula 5′-(C_{A})ₙ₁₀R_{A}-3′, wherein n10 represents the length of said (C_{S})ₙ₁₀ measured by codon, n10 is variable and an integer;

an oligonucleotide represented by the formula 5′-(C_{S})ₙ₁₁R_{S}-3′, wherein n11 represents the length of said (C_{S})ₙ₁₁ measured by codon, n11 is variable and an integer;

an oligonucleotide represented by the formula 5-R_{A}(C_{A})ₙ₁₂-3′, wherein n12 represents the length of said (C_{A})ₙ₁₂ measured by codon, n12 is variable and an integer;

an oligonucleotide represented by the formula 5′-E_{S}(V_{S})ₙ₁₃-3′, wherein n13 represents the length of said (V_{S})ₙ₁₃ measured by codon, n13 is variable and an integer;

an oligonucleotide represented by the formula 5′-(V_{A})ₙ₁₄E_{A}-3′, wherein n14 represents the length of said (V_{A})ₙ₁₄ measured by codon, n14 is variable and an integer;

an oligonucleotide represented by the formula 5′-(V_{S})ₙ₁₅E_{S}-3′, wherein n15 represents the length of said (V_{S})ₙ₁₅ measured by codon, n15 is variable and an integer;

an oligonucleotide represented by the formula 5′-E_{A}(V_{A})ₙ₁₆-3′, wherein n16 represents the length of said (V_{A})ₙ₁₆ measured by codon, n16 is variable and an integer;

an oligonucleotide represented by the formula 5′-(C_{S})ₙ₁₇-3′, wherein n17 represents the length of said (C_{S})ₙ₁₇ measured by codon, n17 is variable and an integer;

an oligonucleotide represented by the formula 5′-(C_{A})ₙ₁₈-3′, wherein n18 represents the length of said (C_{A})ₙ₁₈ measured by codon, n18 is variable and an integer;

an oligonucleotide represented by the formula 5′-(C_{S})ₙ₁₉-3′, wherein each said oligonucleotide further comprises a linker at either 5′-end or 3′-end of said oligonucleotide, the said linker is an amino acid coding codon in sense orientation, n19 represents the length of said (C_{S})ₙ₁₉ measured by codon, n19 is variable and an integer;

an oligonucleotide represented by the formula 5′-(C_{S})ₙ₂₀-3′, wherein each said oligonucleotide further comprises a linker at either 5′-end or 3′-end of said oligonucleotide, the said linker is two consecutive amino acid coding codons in sense orientation, n20 represents the length of said (C_{S})ₙ₂₀ measured by codon, n20 is variable and an integer;

an oligonucleotide represented by the formula 5′-(C_{S})ₙ₂₁-3′, wherein each said oligonucleotide further comprises a linker at either 5′-end or 3′-end of said oligonucleotide, the said linker is three consecutive amino acid coding codons in sense orientation, n21 represents the length of said (C_{S})ₙ₂₁ measured by codon, n21 is variable and an integer;

an oligonucleotide represented by the formula 5′-(C_{S})ₙ₂₂-3′, wherein each said oligonucleotide further comprises a linker at either 5′-end or 3′-end of said oligonucleotide, the said linker is a codon comprising one universal base, n22 represents the length of said (C_{S})ₙ₂₂ measured by codon, n22 is variable and an integer;

an oligonucleotide represented by the formula 5′-(C_{S})ₙ₂₃-3′, wherein each said oligonucleotide further comprises a linker at either 5′-end or 3′-end of said oligonucleotide, the said linker is a codon comprising two universal bases, n23 represents the length of said (C_{S})ₙ₂₃ measured by codon, n23 is variable and an integer;

an oligonucleotide represented by the formula 5′-(C_{S})ₙ₂₄-3′, wherein each said oligonucleotide further comprises a linker at either 5′-end or 3′-end of said oligonucleotide, the said linker is a codon comprising three universal bases, n24 represents the length of said (C_{S})ₙ₂₄ measured by codon, n24 is variable and an integer;

an oligonucleotide represented by the formula 5′-(C_{A})ₙ₂₅-3′, wherein each said oligonucleotide further comprises a linker at either 5′-end or 3′-end of said oligonucleotide, the said linker is an amino acid coding codon in antisense orientation, n25 represents the length of said (C_{A})ₙ₂₅ measured by codon, n25 is variable and an integer;

an oligonucleotide represented by the formula 5′-(C_{A})ₙ₂₆-3′, wherein each said oligonucleotide further comprises a linker at either 5′-end or 3′-end of said oligonucleotide, the said linker is two consecutive amino acid coding codons in antisense orientation, n26 represents the length of said (C_{A})ₙ₂₆ measured by codon, n26 is variable and an integer;

an oligonucleotide represented by the formula 5′-(C_{A})ₙ₂₇-3′, wherein each said oligonucleotide further comprises a linker at either 5′-end or 3′-end of said oligonucleotide, the said linker is three consecutive amino acid coding codons in antisense orientation, n27 represents the length of said (C_{A})ₙ₂₇ measured by codon, n27 is variable and an integer;

an oligonucleotide represented by the formula 5′-(C_{A})ₙ₂₈-3′, wherein each said oligonucleotide further comprises a linker at either 5′-end or 3′-end of said oligonucleotide, the said linker is a codon comprising one universal base, n28 represents the length of said (C_{A})ₙ₂₈ measured by codon, n28 is variable and an integer;

an oligonucleotide represented by the formula 5′-(C_{A})ₙ₂₉-3′, wherein each said oligonucleotide further comprises a linker at either 5′-end or 3′-end of said oligonucleotide, the said linker is a codon comprising two universal bases, n29 represents the length of said (C_{A})ₙ₂₉ measured by codon, n29 is variable and an integer;

an oligonucleotide represented by the formula 5′-(C_{A})ₙ₃₀-3′, wherein each said oligonucleotide further comprises a linker at either 5′-end or 3′-end of said oligonucleotide, the said linker is a codon comprising three universal bases, n30 represents the length of said (C_{A})ₙ₃₀ measured by codon, n30 is variable and an integer;

an oligonucleotide represented by the formula 5′-(V_{S})ₙ₃₁-3′, wherein each said oligonucleotide further comprising a linker at either 5′-end or 3′-end of said oligonucleotide, the said linker is a codon in sense orientation, n31 represents the length of said (V_{S})ₙ₃₁ measured by codon, n31 is variable and an integer;

an oligonucleotide represented by the formula 5′-(V_{S})ₙ₃₂-3′, wherein each said oligonucleotide further comprising a linker at either 5′-end or 3′-end of said oligonucleotide, the said linker is two consecutive codons in sense orientation, n32 represents the length of said (V_{S})ₙ₃₂ measured by codon, n32 is variable and an integer;

an oligonucleotide represented by the formula 5′-(V_{S})ₙ₃₃-3′, wherein each said oligonucleotide further comprising a linker at either 5′-end or 3′-end of said oligonucleotide, the said linker three consecutive codons in sense orientation, n33 represents the length of said (V_{S})ₙ₃₃ measured by codon, n33 is variable and an integer;

an oligonucleotide represented by the formula 5′-(V_{S})ₙ₃₄-3′, wherein each said oligonucleotide further comprising a linker at either 5′-end or 3′-end of said oligonucleotide, the said linker is a codon comprising one universal base, n34 represents the length of said (V_{S})ₙ₃₄ measured by codon, n34 is variable and an integer;

an oligonucleotide represented by the formula 5′-(V_{S})ₙ₃₅-3′, wherein each said oligonucleotide further comprising a linker at either 5′-end or 3′-end of said oligonucleotide, the said linker is a codon comprising two universal bases, n35 represents the length of said (V_{S})ₙ₃₅ measured by codon, n35 is variable and an integer;

an oligonucleotide represented by the formula 5′-(V_{S})ₙ₃₆-3′, wherein each said oligonucleotide further comprising a linker at either 5′-end or 3′-end of said oligonucleotide, the said linker is a codon comprising three universal bases, n36 represents the length of said (V_{S})ₙ₃₆ measured by codon, n36 is variable and an integer;

an oligonucleotide represented by the formula 5′-(V_{S})ₙ₃₇-3′, wherein each said oligonucleotide further comprising a linker at either 5′-end or 3′-end of said oligonucleotide, the said linker is a codon comprising one LNA, n37 represents the length of said (V_{S})ₙ₃₇ measured by codon, n37 is variable and an integer;

an oligonucleotide represented by the formula 5′-(V_{S})ₙ₃₈-3′, wherein each said oligonucleotide further comprising a linker at either 5′-end or 3′-end of said oligonucleotide, the said linker is a codon comprising two LNAs, n38 represents the length of said (V_{S})ₙ₃₈ measured by codon, n38 is variable and an integer;

an oligonucleotide represented by the formula 5′-(V_{S})ₙ₃₉-3′, wherein each said oligonucleotide further comprising a linker at either 5′-end or 3′-end of said oligonucleotide, the said linker is a codon comprising three LNAs, n39 represents the length of said (V_{S})ₙ₃₉ measured by codon, n39 is variable and an integer;

an oligonucleotide represented by the formula 5′-(V_{S})ₙ₄₀-3′, wherein each said oligonucleotide further comprising a linker at either 5′-end or 3′-end of said oligonucleotide, the said linker is a codon comprising one MF, n40 represents the length of said (V_{S})ₙ₄₀ measured by codon, n40 is variable and an integer;

an oligonucleotide represented by the formula 5′-(V_{S})ₙ₄₁-3′, each said oligonucleotide further comprising a linker at either 5′-end or 3′-end of said oligonucleotide, the said linker is a codon comprising two MFs, n41 represents the length of said (V_{S})ₙ₄₁ measured by codon, n41 is variable and an integer;

an oligonucleotide represented by the formula 5′-(V_{S})ₙ₄₂-3′, wherein each said oligonucleotide further comprising a linker at either 5′-end or 3′-end of said oligonucleotide, the said linker is a codon comprising three MFs, n42 represents the length of said (V_{S})ₙ₄₂ measured by codon, n42 is variable and an integer;

an oligonucleotide represented by the formula 5′-(V_{S})ₙ₄₃-3′, wherein each said oligonucleotide further comprising a linker at either 5′-end or 3′-end of said oligonucleotide, the said linker is a codon comprising one PNA, n43 represents the length of said (V_{S})ₙ₄₃ measured by codon, n43 is variable and an integer;

an oligonucleotide represented by the formula 5′-(V_{S})ₙ₄₄-3′, wherein each said oligonucleotide further comprising a linker at either 5′-end or 3′-end of said oligonucleotide, the said linker is a codon comprising two PNAs, n44 represents the length of said (V_{S})ₙ₄₄ measured by codon, n44 is variable and an integer;

an oligonucleotide represented by the formula 5′-(V_{S})ₙ₄₅-3′, wherein each said oligonucleotide further comprising a linker at either 5′-end or 3′-end of said oligonucleotide, the said linker is a codon comprising three PNAs, n45 represents the length of said (V_{S})ₙ₄₅ measured by codon, n45 is variable and an integer;

an oligonucleotide represented by the formula 5′-(V_{S})ₙ₄₆-3′, wherein each said oligonucleotide further comprising a linker at either 5′-end or 3′-end of said oligonucleotide, the said linker is a codon comprising one 2′-MOE, n46 represents the length of said (V_{S})ₙ₄₆ measured by codon, n46 is variable and an integer;

an oligonucleotide represented by the formula 5′-(V_{S})ₙ₄₇-3′, wherein each said oligonucleotide further comprising a linker at either 5′-end or 3′-end of said oligonucleotide, the said linker is a codon comprising two 2′-MOEs, n47 represents the length of said (V_{S})ₙ₄₇ measured by codon, n47 is variable and an integer;

an oligonucleotide represented by the formula 5′-(V_{S})ₙ₄₈-3′, wherein each said oligonucleotide further comprising a linker at either 5′-end or 3′-end of said oligonucleotide, the said linker is a codon comprising three 2′-MOEs, n48 represents the length of said (V_{S})ₙ₄₈ measured by codon, n48 is variable and an integer;

an oligonucleotide represented by the formula 5′-(V_{S})ₙ₄₉-3′, wherein each said oligonucleotide further comprising a linker at either 5′-end or 3′-end of said oligonucleotide, the said linker is a codon comprising one PS, n49 represents the length of said (V_{S})ₙ₄₉ measured by codon, n49 is variable and an integer;

an oligonucleotide represented by the formula 5′-(V_{S})ₙ₅₀-3′, wherein each said oligonucleotide further comprising a linker at either 5′-end or 3′-end of said oligonucleotide, the said linker is a codon comprising two PSs, n50 represents the length of said (V_{S})ₙ₅₀ measured by codon, n50 is variable and an integer;

an oligonucleotide represented by the formula 5′-(V_{S})ₙ₅₁-3′, wherein each said oligonucleotide further comprising a linker at either 5′-end or 3′-end of said oligonucleotide, the said linker is a codon comprising three PSs, n51 represents the length of said (V_{S})ₙ₅₁ measured by codon, n51 is variable and an integer;

an oligonucleotide represented by the formula 5′-oligo-d(T)_{S}-3′, wherein the length of said d(T)_{S} is measured by nucleotide, the said s is variable and an integer, the value of the said s is from 21 to 6; and combinations thereof.

According to each said formula, each of said oligonucleotide panels comprise substantially all of said oligonucleotides. According to each said formula, each of said oligonucleotide panels consist essentially of said oligonucleotides.

As will be appreciated by one of skilled in the art, n1 to n46 individually may be any positive, non-zero integer. That is, within a given kit or panel, n1 may be 3 and n2 may be 2; alternatively, for example, both n1 and n2 may be 2. In other embodiments, n1 to n46 may individually be an integer from 1-8, from 1-7, from 1-6, from 1-5 or from 1-4. As will be appreciated by one of skilled the art, a given single panel may consist of 2 or more sets of sense oligonucleotides or antisense oligonucleotides of one of the above-described formulae; 5 or more sets of sense oligonucleotides or antisense oligonucleotides of one of the above-described formulae; 10 or more sets of sense oligonucleotides or antisense oligonucleotides of one of the above-described formulae; 15 or more sets of sense oligonucleotides or antisense oligonucleotides of one of the above-described formulae; 20 or more sets of sense oligonucleotides or antisense oligonucleotides of one of the above-described formulae; 25 or more sets of sense oligonucleotides or antisense oligonucleotides of one of the above-described formulae; or 50 or more sets of sense oligonucleotides or antisense oligonucleotides of one of the above-described formulae; 100 or more sets of sense oligonucleotides or antisense oligonucleotides of one of the above-described formulae; or 200 or more sets of sense oligonucleotides or antisense oligonucleotides of one of the above-described formulae; 300 or more sets of sense oligonucleotides or antisense oligonucleotides of one of the above-described formulae; or 500 or more sets of sense oligonucleotides or antisense oligonucleotides of one of the above-described formulae; 1,000 or more sets of sense oligonucleotides or antisense oligonucleotides of one of the above-described formulae; or 2,000 or more sets of sense oligonucleotides or antisense oligonucleotides of one of the above-described formulae; 3,000 or more sets of sense oligonucleotides or antisense oligonucleotides of one of the above-described formulae; or 5,000 or more sets of sense oligonucleotides or antisense oligonucleotides of one of the above-described formulae; 10,000 or more sets of sense oligonucleotides or antisense oligonucleotides of one of the above-described formulae; or 20,000 or more sets of sense oligonucleotides or antisense oligonucleotides of one of the above-described formulae; 50,000 or more sets of sense oligonucleotides or antisense oligonucleotides of one of the above-described formulae; or 100,000 or more sets of sense oligonucleotides or antisense oligonucleotides of one of the above-described formulae; 200,000 or more sets of sense oligonucleotides or antisense oligonucleotides of one of the above-described formulae; or 500,000 or more sets of sense oligonucleotides or antisense oligonucleotides of one of the above-described formulae; and in one preferred embodiment, the said GC Identical Panels could be further sub-classified according to GC content after the incorporation of LNA. In another preferred embodiment, the Tm of the said GC Identical Panels could be further adjusted by the incorporation of appropriate number of LNA. In other embodiments, the said GC Identical Panels could be adjusted by the incorporation of appropriate number of LNA. In other embodiments, the said GC Identical Panels could be adjusted by the incorporation of appropriate number of MF. In other embodiments, the said GC Identical Panels could be adjusted by the incorporation of appropriate number of PNA. In other embodiments, the said GC Identical Panels could be adjusted by the incorporation of appropriate number of 2′-MOE. In other embodiments, the said GC Identical Panels could be adjusted by the incorporation of appropriate number of PS.

In yet other embodiments, a panel may comprise substantially all of the sense oligonucleotides or antisense oligonucleotides of one of the above-described formulae. In one another embodiments, a panel may consist essentially of said sense oligonucleotides or antisense oligonucleotides according to one of the above-described formulae. In other embodiments of the invention, each sense oligonucleotides or of antisense oligonucleotides of the panel may consist essentially of an sense oligonucleotides or antisense oligonucleotides according to the specific formula for the respective panel, as discussed herein and hereinafter.

According to a derivative aspect of the invention, there is a kit(s) provided for identifying targeting antibodies within a sample comprising at least one of the following:

a N-terminal restriction endonuclease peptide panel comprising a plurality of peptides, wherein each of the peptides is represented by the formula N-terminal-R_{E}(A)ₙ₃₈-C-terminal, wherein n38 represents the length of (A)ₙ₃₈ measured by amino acid, n38 is variable and an integer;

a C-terminal restriction endonuclease peptide panel comprising a plurality of peptides, wherein each of the peptides is represented by the formula N-terminal-(A)ₙ₃₉R_{E}-C-terminal, wherein n39 represents the length of (A)ₙ₃₉ measured by amino acid, n39 is variable and an integer;

a N-terminal peptide panel comprising a plurality of peptides, wherein each of the peptides is represented by the formula N-terminal-M(A)ₙ₄₀-C-terminal, wherein n40 represents the length of (A)ₙ₄₀ measured by amino acid, n40 is variable and an integer;

a C-terminal peptide panel comprising a plurality of peptides, wherein each of the peptides is represented by the formula N-terminal-(A)ₙ₄₁-C-terminal, wherein n41 represents the length of (A)ₙ₄₁ measured by amino acid, n41 is variable and an integer;

a peptide panel comprising a plurality of peptides, wherein each of the peptides is represented by the formula N-terminal-(A)ₙ₄₂-C-terminal, wherein each said peptide further comprises a linker at neither N-terminal or C-terminal of said peptide, the said linker being is an amino acid encoded by an initiation codon, wherein n42 represents the length of (A)ₙ₄₂ measured by amino acid, n42 is variable and an integer;

a peptide panel comprising a plurality of peptides, wherein each of the peptides is represented by the formula N-terminal-(A)ₙ₄₃-C-terminal, wherein each said peptide further comprises a linker at neither N-terminal or C-terminal of said peptide, the said linker being is an amino acid encoded by a codon, wherein n43 represents the length of (A)ₙ₄₃ measured by amino acid, n43 is variable and an integer;

a peptide panel comprising a plurality of peptides, wherein each of the peptides is represented by the formula N-terminal-(A)ₙ₄₄-C-terminal, wherein each said peptide further comprises a linker at neither N-terminal or C-terminal of said peptide, the said linker being is two consecutive amino acids encoded by two codons, wherein n44 represents the length of (A)ₙ₄₄ measured by amino acid, n44 is variable and an integer;

a peptide panel comprising a plurality of peptides, wherein each of the peptides is represented by the formula N-terminal-(A)ₙ₄₅-C-terminal, wherein each said peptide further comprises a linker at neither N-terminal or C-terminal of said peptide, the said linker being is two consecutive amino acid deduced from a two codon restriction endonuclease recognition site, wherein n45 represents the length of (A)ₙ₄₅ measured by amino acid, n45 is variable and an integer;

a peptide panel comprising a plurality of peptides, wherein each of the peptides is represented by the formula N-terminal-(A)ₙ₄₆-C-terminal, wherein each said peptide further comprises a linker at neither N-terminal or C-terminal of said peptide, the said linker being is three two consecutive amino acids encoded by three codons, wherein n46 represents the length of (A)ₙ₄₆ measured by amino acid, n46 is variable and an integer; and combinations thereof.

### DESCRIPTION OF THE PREFERRED EMBODIMENTS

Unless defined otherwise, all technical and scientific terms used herein have the same meaning as commonly understood by one of ordinary skilled in the art to which the invention belongs. Although any methods and materials similar or equivalent to those described herein can be used in the practice or testing of the present invention, the preferred methods and materials are now described. All publications mentioned hereunder are incorporated herein by reference.

### Definitions

“Oligonucleotide” refers to polymeric forms of nucleotides of a given length of a given single-stranded nucleic acid molecule which include sense strand and antisense strand. As used herein, the length of oligonucleotide is preferably measured by codon. In general, the length is at least one codon long, or preferably at least two, three, four, five, six, seven, eight, nine or ten codons long but preferably no more than ten codons long. As will be appreciated by one of skilled in the art, oligonucleotide includes deoxyribonucleotides (DNAs), ribonucleotides (RNAs) and their corresponding analogs and derivatives thereof. For example, Locked Nucleic Acid (LNA), Peptide Nucleic Acid (PNA), Morpholino phosphoroamidate (MF), 2′-O-Methoxyethyl oligonucleotide(s) (2′-MOE), 2′-O-Methyl (2′-OME), Phosphorothioate (PS), Phosphoroamidate, Methylphosphonate and Universal base belong to the said analogs and derivatives. Oligonucleotides include all formats of chemical modifications or and substitutions, which are both on and in between of nucleotides within a given oligonucleotide. One ordinary skilled in the relevant art would recognize that said chemical modifications and substitutions include but are by no means limited to chemical modifications or substitutions on the molecular structures of pentose sugar, phosphate group and nitrogenous base of said oligonucleotides. For example, methylation of the naturally occurring nucleotides and analogs is one of the formats of chemical modifications. Modifications of internucleotide linkages, for example but by no means limited to phosphonates, methyl phosphonates, phosphoroamidites, phosphotriesters, phosphorothioates, phosphorodithioates, 2′-5′ linkages, non-phosphorus linkages and the like are included. One ordinary skilled in the relevant art would recognize that said chemical modifications and substitutions include but are by no means limited to chimeric oligonucleotides. Oligonucleotides may be labeled with radio isotopes, for example, .sup.32P or .sup.33P or .sup.35S or the like. Alternatively oligonucleotides may be labeled with other molecules that provide a detectable signal, either directly or indirectly, for example but by no means limited to fluorescent dyes, biotin, digoxigenin, alkaline phosphatase and the like.

“Antisense oligonucleotide” refers to polymeric forms of nucleotides of a given length of a single antisense stranded of nucleic acid molecule. As used herein, the length of antisense oligonucleotide is preferably measured by antisense codon. In general, the length is at least one antisense codon long, or preferably at least two, three, four, five, six, seven, eight, nine or ten antisense codons long but preferably no more than ten antisense codons long. As will be appreciated by one of skilled in the art, antisense oligonucleotide includes deoxyribonucleotides (DNAs), ribonucleotides (RNAs) and their corresponding analogs and derivatives thereof. For example, Locked Nucleic Acid (LNA), Peptide Nucleic Acid (PNA), Morpholino phosphoroamidate (MF), 2′-O-Methoxyethyl oligonucleotide(s) (2′-MOE), 2′-O-Methyl (2′-OME), Phosphorothioate (PS), Phosphoroamidate, Methylphosphonate and Universal base belong to the said analogs and derivatives. Antisense oligonucleotides include all formats of chemical modifications or and substitutions, which are both on and in between of nucleotides within a given antisense oligonucleotide. One ordinary skilled in the relevant art would recognize that said chemical modifications and substitutions include but are by no means limited to chemical modifications or substitutions on the molecular structures of pentose sugar, phosphate group and nitrogenous base of said antisense oligonucleotides. For example, methylation of the naturally occurring nucleotides and analogs is one of the formats of chemical modifications. Modifications of internucleotide linkages, for example but by no means limited to phosphonates, methyl phosphonates, phosphoroamidites, phosphotriesters, phosphorothioates, phosphorodithioates, 2′-5′ linkages, non-phosphorus linkages and the like are included. One ordinary skilled in the relevant art would recognize that said chemical modifications and substitutions include but are by no means limited to chimeric oligonucleotides. Antisense oligonucleotides may be labeled with radio isotopes, for example, .sup.32P or .sup.33P or .sup.35S or the like. Alternatively antisense oligonucleotides may be labeled with other molecules that provide a detectable signal, either directly or indirectly, for example but by no means limited to fluorescent dyes, biotin, digoxigenin, alkaline phosphatase and the like.

“Sequence of orientation” refers to a selected sense sequence or known sense sequence for the orientation of the entire sense sequence which is measured by codon or expressed codon (essential amino acid). For a non-limiting example, 5′-AGC in 5′-AGCGCACTC is sequence of orientation or known sequence which is a selected sequence for orientation of the entire sense sequence of 5′-AGCGCACTC, wherein n represents the length of the sense sequence measured by codon, wherein m represents the length of the sense sequence of orientation measured by codon, wherein n=3, wherein m=1, wherein n−m=2.

“Antisense sequence of orientation” refers to a selected antisense sequence or known antisense sequence for orientation of the entire antisense sequence which is measured by antisense codon. For a non-limiting example, GCT-3′ in 5′-GTGTGCGCT-3′ is the antisense sequence of orientation or known antisense sequence which is a selected antisense sequence for orientation of the entire antisense sequence of 5′-GTGTGCGCT-3′, wherein n represents the length of the antisense sequence measured by antisense codon, wherein m represents the length of the antisense sequence of orientation measured by antisense codon, wherein n=3, wherein m=1, wherein n−m=2.

“Panel” refers to a plurality of reagents, for example, oligonucleotides or antisense oligonucleotides. The panel may be immobilized or linked or associate or attached or integrated to a carrier for delivery such as Lentiviruses, Adenoviruses, lipidoids, amphoteric liposomes, nanoparticles such as chitosan nanoparticles and other suitable carriers for antisense oligonucleotide delivery known in the art. In a set of each said oligonucleotide or antisense oligonucleotide, the said set comprising at least two copies of the said oligonucleotide or antisense oligonucleotide. The said oligonucleotide or antisense oligonucleotide comprises at least two said sets. The panels may be used alone or in combination. The said oligonucleotide or antisense oligonucleotide panels comprise substantially all of said oligonucleotides or antisense oligonucleotide. According to each said formula, each of said oligonucleotide panels consist essentially of said oligonucleotides or antisense oligonucleotides. The entire panel or individual oligonucleotides or antisense oligonucleotide thereof may be in a substantially aqueous phase.

“Set” refers to an organizational format for a plurality of reagents, such as oligonucleotides or antisense oligonucleotides on a panel. Each set has at least two copies of an oligonucleotide or antisense oligonucleotide. Usually, each set possesses at least more than two copies of an oligonucleotide or more than two copies of an antisense oligonucleotide. In some embodiments, each of the said set may have at least two copies of one distinctive oligonucleotide or antisense oligonucleotide. In some embodiments, each of the said distinctive oligonucleotide or antisense oligonucleotide in a set has the identical length. In some embodiments, all the said distinctive oligonucleotides or antisense oligonucleotides of all the sets of the entire panel may have the identical length. In other embodiments, all the said distinctive oligonucleotides or antisense oligonucleotides of all the sets of the entire panel may have both the identical length and GC content.

“GC Identical Panel” refers to a format of an oligonucleotide or antisense oligonucleotide panel. The GC Identical Panel consists of sets of oligonucleotides or antisense oligonucleotides that are all identical in GC content. In one preferred embodiment, none of the oligonucleotide or antisense oligonucleotide sequences of a set are identical to other sets within a given panel; but the said oligonucleotide or antisense oligonucleotide sequences are all identical in GC content in each set within a panel. In another preferred embodiment, none of the oligonucleotide or antisense oligonucleotide sequences of a set are identical to other sets within a given panel, but the said oligonucleotide or antisense oligonucleotide sequences are all identical in GC content and length in each set within a panel.

“Genetic signature” or “marker” refers to a biological characteristic of, for example, a gene, mRNA, peptide, an ORF sequence, a nucleic acid sequence, a peptide sequence, antigen, antibody, cell, cell line, tissue, organ, individual or organism. Examples of genetic signatures or marker include but are by no means limited to locations and the immediate adjacent regions of start and stop codons within a gene, locations and the immediate adjacent regions of restriction enzyme sites within a gene, locations and the immediate adjacent regions of promoter sequences within a gene, presence of antigens of a specific amino acid sequence, presence of antibodies recognizing a specific amino acid sequence in a sample, expression pattern(s) or expression fingerprint(s) or expression profile(s) of mRNA(s), cDNA(s), gene(s), genome, peptide(s), Protein(s), cell(s), cell line(s) and the like.

“Hybridization” refers to an interaction between two strands of nucleic acids by hydrogen bonds in accordance with the rules of Watson-Crick DNA complementarity, Hoogstein binding, or other sequence-specific binding known in the art. Hybridization can be performed under different stringent hybridization conditions known in the art. Under appropriate stringent conditions, hybridization between the two complementary strands could reach at 60% or above, 61% or above, 62% or above, 63% or above, 64% or above, 65% or above, 66% or above, 67% or above, 68% or above, 69% or above, 70% or above, 71% or above, 72% or above, 73% or above, 74% or above, 75% or above, 76% or above, 77% or above, 78% or above, 79% or above, 80% or above, 81% or above, 82% or above, 83% or above, 84% or above, 85% or above, 86% or above, 87% or above, 88% or above, 89% or above, 90% or above, 91% or above, 92% or above, 93% or above, 94% or above, 95% or above, 96% or above, 97% or above, 98% or above, 99% or above in the reactions. For a non-limiting example, one of the said stringent conditions is hybridization in 6× Sodium Chloride/Sodium Citrate (SCC) at 42° C. 12 hrs. in water bath; subsequently being washed twice by 0.2×SSC, 0.1% SDS solution at 50° C. in water bath for 30 minutes and being final washed three times by 0.1×SSC, 0.1% SDS solution at 65° C. in water bath for 30 minutes.

“Substantially all” refers to the fact that a sufficient number of individuals or sets or groups or panels are present that the desired result can be obtained or determined. For example, regarding the use of an antisense oligonucleotide library, “substantially all” members of a specific formula means that enough of the respective antisense oligonucleotides represented by the specific formula are present in the library such that it is a reasonable prediction that the desired result may be obtained. Examples of suitable desired results are discussed in detail herein. As will be appreciated by one of skilled in the art, the exact value of “substantially all” is context dependent and shall of course depend on many factors, such as how the library is being used, the length of the antisense oligonucleotides, the GC content and Tm of antisense oligonucleotides, the way of Tm adjustment and how the material being screened as well as other factors. “Substantially all” may be for example 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or 99.99% of the antisense oligonucleotides represented by a specific formula.

“Consisting essentially of” means that the described molecules consist of those oligonucleotides or antisense oligonucleotides as described in the formulae listed as well as other components which are in the scope and spirit of the invention. As will be appreciated by one of skilled in the art, in the case of the antisense oligonucleotides, examples include but are by no means limited to antisense oligonucleotides containing Universal base or Locked Nucleic Acid (LNA) or Peptide Nucleic Acid (PNA) or Morpholino phosphoroamidate (MF) or 2′-O-Methoxyethyl oligonucleotide(s) (2′-MOE) or 2′-O-Methyl (2′-OME) or Phosphorothioate (PS) or Phosphoroamidate or Methylphosphonate or other chemical modified oligonucleotide(s) as described herein.

“Discrete” in regards the positioning of an oligonucleotide or antisense oligonucleotide on a carrier refers to the fact that the oligonucleotide or antisense oligonucleotide or set thereof is positioned such that a signal therefore can be detected unambiguously. As will be appreciated by one of skilled in the art, what is and isn't a discrete position shall depend largely on the reporting signal used, the platform and the detection method as well as other factors well known to one of skilled in the art.

“Plurality” refers to 2 or more.

“Strands of the Double Helix of Nucleic Acids” refers to two strands of each helix of nucleic acids: A sense strand is often termed as a non-template strand or coding strand whereas, an anti-sense strand is often termed as a template strand or non-coding strand.

“Any codon” refers to any one of the 64 nucleotide triplets of the genetic code.

“Any antisense codon” refers to any one of the antisense corresponding sequence counterpart of the 64 nucleotide triplets of the genetic code.

“Sense” orientation or strand refers to the coding strand or the complementary strand of the non-coding strand or the complementary strand of the antisense strand or the non-template strand of a double stranded DNA molecule. The initial sense strand is the strand of DNA transcribed into pre-mRNA strand. The pre-mRNA strand undergoes intron deletion prior to become mRNA strand.

“Antisense” orientation or strand refers to the non-coding strand or complementary strand of the coding strand or complementary strand of the initial sense strand or the template strand of a double-stranded DNA molecule. The antisense strand is the template for pre-mRNA strand or and mRNA strand synthesis.

“Antisense amino acid coding codon” refers to an antisense codon complementary to a codon which encodes an amino acid. In most cases, 61 codons encode for the 20 essential amino acids. In accordance with Watson-Crick DNA complementary rule, the said 61 codons have corresponding 61 antisense codons. As an example, 5′-AGG is a sense codon which codes for arginine. The corresponding antisense codon is 5′-CCT. In mammalian mitochondria, there are specific 60 codons encode for the 20 essential amino acids and 60 antisense corresponding codons as the counterpart.

“Antisense initiation codon” refers to an antisense codon complementary to a codon that may function as the start codon. In most cases, the sense initiation codon is 5′-ATG; the antisense initiation codon is 5′-CAT. As discussed herein, other initiation codons may be used in the invention, for example, 5′-ATA, which is the start codon in mammalian mitochondria. Other initiation codons include but are by no means limited to 5′-GTG, 5′-ATA, 5′-TTG, 5′-ACG and 5′-CTG.

“Antisense termination codon” refers to an antisense codon complementary to a codon that may function as the stop codon. In most cases, there are three major sense stop codons: 5′-TAA, 5′-TGA and 5′-TAG. There are three major corresponding antisense stop codons: 5′-TTA, 5′-TCA and 5′-CTA. As discussed herein, other sense termination codons and their corresponding antisense termination codons may be used in the invention, for example, 5′-AGA, 5′-AGG, 5′-TAA/5′-UAA and 5′-TAG/5′-UAG, which are the sense stop codons in mammalian mitochondria.

“Locked Nucleic Acids” refers to but is by no means limited to an oligonucleotide that contains one or more 2′-O, 4′-methylene-beta-D-robofuranosyl nucleotide monomer(s) which is a member of Locked Nucleic Acids (LNA) family. LNA is water soluble. It possesses increasing thermal stability, mismatch discriminating capacity and high affinity towards complementary DNA and RNA molecules. It improves the performance of short PCR primer, sense and antisense oligonucleotide significantly.

“Universal base” refers to molecules capable of substituting for binding to any one of A, C, G, T and U in nucleic acids by forming hydrogen bonds without significant structure destabilization. The oligonucleotide incorporated with the universal base analogues is able to function as a probe in hybridization, as a primer in PCR and DNA sequencing. Examples of universal bases include but are by no means limited to 5′-nitroindole-2′-deoxyriboside, 3-nitropyrrole, inosine and pypoxanthine.

“Oligo-d(T)_{S}” refers to a plurality of consecutive thymidine nucleotides represented by the formula 5′-oligo-d(T)_{S}-3′, wherein the length of said d(T)_{S} is measured by nucleotide, the said s is a variable and integer, the value of the said s is from 30 to 6. The length of 5′-oligo-d(T)_{S}-3′ could be measured by 5′-TTT as well.

The present invention provides a general universal genetic algorithm, from which stems a series of universal genetic algorithms. It provides a universal calculation formula for the total number of sense and antisense sequences at a given length measured by either the number of codon or antisense codon or L-amino acid encoded by codon as the unit when the strand and the orientation for a sequence have been determined. There are two strands of DNA and RNA, namely sense strand and antisense strand. The orientation for a sequence of a DNA or RNA could be located at either 5′-end or 3′-end of its sequence. The orientation for a peptide sequence, the product of a gene, could be located at either N-terminal or C-terminal of its sequence. The length of the sequence is measured by codon. The length measurement could be converted to the measurement unit of single nucleotide by multiplying by 3.

The algorithms are applicable to sense and antisense strands of a gene and all the corresponding gene products, such as mRNA, cDNA, antisense RNA, antisense cDNA, peptide and protein. The general universal genetic algorithm is presented herein:

Y=X.sup.(n−m)

1. Definition of X

Nucleic Acids:

(1) Sense Strand:
X: The number of all distinct codons. X is a variable. X is an integer. X is not equal zero.
X is from 1 to infinity. At the current evolutionary stage: For all distinct codons, X=64. For all distinct codons that encode L-amino acid, X=61.

(2) Antisense Strand:
X: The number of all distinct antisense codons. X is a variable. X is an integer. X is not equal zero. X is from 1 to infinity. At the current evolutionary stage: For all distinct antisense codons, X=64. For all distinct antisense L-amino acid codons, X=61.

Peptides:
X: The number of all distinct L-amino acids encoded by at least one codon. X is a variable. X is an integer. X is not equal zero. X is from 1 to infinity. At the current evolutionary stage: 20 distinct essential L-amino acids that are encoded by 61 distinct corresponding codons. X=20.

2. Definition of n

Nucleic Acids:

(1) Sense Strand:
n: number of all codons arranged linearly without overlapping per sense sequence including selected sense sequence of orientation (m) in within. Sense sequence of orientation is a selected sense sequence. n is a variable. n is an integer. n is not equal zero. n<infinity. n represents the entire length of sense sequence measured by codon (triplet of nucleotides). n represents serial numbers of codons counted from either 5′-end or 3′-end of the sense sequence.

(2) Antisense Strand:
n: number of all antisense codons arranged linearly without overlapping per antisense sequence including selected antisense sequence of orientation (m) in within. Antisense sequence of orientation is a selected antisense sequence. n is a variable. n is an integer. n is not equal zero. n<infinity. n represents the entire length of the antisense sequence measured by antisense codon. n represents serial numbers of antisense codons counted from either 5′-end or 3′-end of the antisense sequence.

Peptides:
n: number of all L-amino acids arranged linearly without overlapping per sequence including selected sequence of orientation (m) in within. n is a variable. n is an integer. n is not equal zero. n<infinity. n represents the length of sequence measured by L-amino acids encoded by codons. n represents the number of amino acids counted from either N-terminal or C-terminal of a peptide or protein sequence.

3. Definition of m

Nucleic Acids:

(1) Sense Strand:
m: number of all codons of sense sequence of orientation located at either 5′-end or 3′-end of the entire sense sequence. The sense sequence of orientation is a selected sense sequence. For example, if there is no sequence of orientation located at either 5′-end or 3′-end of the entire sense sequence, m=zero. If a sense sequence started from adjacent downstream to 5′-ATG in 5′ to 3′ direction, m=1. If a sense sequence started from adjacent upstream to 3′-AGT (5′-TGA) in 3′ to 5′ direction, m=1. If a sense sequence started from adjacent downstream to 5′-GAATTC (EcoR I recognition sense sequence) in 5′ to 3′ direction, m=2. If a sense sequence started from adjacent downstream to 5′-CACACAGGAGAAAAGCCA (SEQ ID No. 12) (sense conservative motif of six amino acids of a zinc finger gene family) in 5′ to 3′ direction, m=6. m is a variable. m is from zero to n. m<n. m is an integer.

(2) Antisense Strand:
m: number of all antisense codons of antisense sequence of orientation located at either 5′-end or 3′-end of the entire antisense sequence. The antisense sequence of orientation is a selected antisense sequence. For example, if there is no antisense sequence of orientation located at either 5′-end or 3′-end of the beginning of the entire antisense sequence, m=zero. If an antisense sequence started from adjacent upstream to 3′-TAC in 3′ to 5′ direction, m=1. If an antisense sequence started from adjacent downstream to 3′-ACT (5′-TCA) in 5′ to 3′ direction, m=1. If an antisense sequence started from adjacent upstream to 5′-GAATTC (EcoR I recognition antisense sequence) in 3′ to 5′ direction, m=2. If an antisense sequence started from adjacent upstream to 5′-TGGCTTTTCTCCTGTGTG (SEQ ID No. 13) (antisense conservative motif of six amino acids of a zinc finger gene family) in 3′ to 5′ direction, m=6. m is a variable. m is from zero to n. m<n. m is an integer.

Peptides:
m: number of all amino acids of sequence of orientation per entire sequence located at either N-terminal or C-terminal. For example, if there is no sequence of orientation located at either IN-terminal or C-terminal of the entire sequence, m=zero. if a sequence started from adjacent downstream to an amino acid encoded by a start codon, such as Methionine encoded by 5′-ATG, in N-terminal to C-terminal direction m=1. If a sequence started adjacent upstream to from one amino acid encoded by a codon in C-terminal to N-terminal direction, m=1. If a sequence started from adjacent downstream to N-EF (two amino acids encoded by EcoR I recognition sequence) in N-terminal to C-terminal direction, m=2. If a sequence started from adjacent downstream to NH₂—HTGEFP (SEQ ID No. 14) (conservative motif of six amino acids of zinc finger gene family) in N-terminal to C-terminal direction, m=6. m is a variable. m is from zero to n. m<n. m is an integer.

With knowledge of each of the 64 codons and 20 L-amino acids, the inventive universal genetic algorithm of Y=X.sup.(n−m) provides a quantitative vehicle to deduce all possible sequence(s) of either nucleic acid or peptide of a given length. Starting with the universal genetic algorithm, a series of genetic algorithms have been derived therefrom, as discussed herein. It provides a universal calculation formula for the total number of sequences of sense strand, antisense strand of nucleic acids and peptides of a given length measured by either codon or antisense codon or L-amino acid encoded by codon when the orientation direction has been determined. The length measured by codons can convert to the length measured by single nucleotides by multiplying three (×3). The inventive methodologies are codon-based, which selectively exclude nonsense codons that do not exist in the ORF sequence in the designing oligonucleotide sequences. A series of libraries, such as oligonucleotide probe libraries have been established accordingly as presented herein. The said oligonucleotides can be utilized in reactions in aqueous phases, such as RT-PCR, PCR, Touchdown PCR and Real-time PCR or on the surface of solid phases, such as DNA Microarrays, Dot and filter hybridizations. To address a specific problem of gene expression and regulations, the above mentioned libraries could be used alone or and in combination. The above mentioned libraries could be integrated or and included into a singular product or and in one method.

Generic Library Construction

Each of the distinct polydeoxyoligonucleotides or polyoligonucleotides thereafter of a given length that was measured by the number of codons are linear polymers of molecules covalently joined by deoxynucleotides or nucleotides respectively. Each of the distinct deoxyoligonucleotides is covalently joined together with each other by phosphodiester bonds between 3′-hydroxyl group of the preceding nucleotide and 5′-phosphate group of the immediately adjacent nucleotide in 5′ towards 3′ orientation. The same is true for the oligonucleotides.

Each of the distinct polydeoxyoligonucleotides or polyoligonucleotides thereafter of a given length that was measured by the number of codons was being produced a corresponding antisense polydeoxyoligonucleotides or polyoligonucleotides that was measured by the number of antisense codons in accordance with Watson-Crick DNA complementary rule and vice versa.

Each of the distinct polydeoxyoligonucleotides or polyoligonucleotides thereafter of a given length that was measured by the number of codons was being translated into corresponding expressed-codon-based peptides in accordance with Central Dogma, which consist of L-amino acids. Each of the distinct L-amino acids of the translated peptides is covalently joined together with each other by peptide bonds between carboxylic acid groups of the preceding amino acid and amino groups of the immediately adjacent amino acid in N-terminal towards C-terminal orientation.

Each of the distinct translated peptides thereafter is used as a distinct antigen in the production of the primary specific monoclonal or multiclonal antibodies respectively. Each of the distinct monoclonal or multiclonal antibodies produced by using each distinct translated peptide is used as a distinct antigen in the production of the secondary specific monoclonal and multiclonal antibodies respectively.

Generic ORF Oligonucleotide Libraries with 5′-Start Codon Orientation

For example, at each 5′ end of the most ORF sequences, 5′-ATG occupies the first codon position which orients the entire ORF sequence from 5′ towards 3′. The second codon position in succession of ORF sequence is occupied by one of the 61 codons. The third codon position in succession of the ORF sequence is occupied by one of the 61 codons as well as each of the subsequent sequential codon positions in 5′ towards 3′ direction thereafter. The numbers of the distinctive 5′-ATG oriented ORF sequences increase quantitatively with increasing length. The said numbers could be calculated as long as the specific length (n) and (m) were given according to algorithm of 61.sup.(n−m). In one embodiment, 9-mer 5′-ATG oriented ORF sequence is three-codon-length long. 5′-ATG is selected one-codon-length-long sequence of orientation. Therefore, n=3, m=1, E=n−m. E is exponent. 61.sup.(3-1)=3,721. The total numbers of distinctive 9-mer 5′-ATG oriented ORF sequences are 3,721. When n=6, m=1, (n−m)=5. The n^{th} codon occupies nucleotide positions (3n-2) to (3n) in 5′-ATG oriented n-codon-length-long sequence. Each of nucleotide positions of the n^{th} codon in 5′-oriented triplet format is (3n−2), (3n−1) and (3n) respectively.

In one preferred embodiment, a collection of all 3,721 distinctive 9-mer 5′-ATG oriented ORF sequences has formed a generic 9-mer oligonucleotide library, which is capable to be used as a generic and all-purpose 9-mer oligonucleotide ingredient, probe and primer library. In another preferred embodiment, according to each said formula, each of said oligonucleotide library comprises substantially all of said oligonucleotides. In yet another preferred embodiment, according to each said formula, each of said oligonucleotide library consists essentially of said oligonucleotides. In another preferred embodiment, a collection of above 3,721 distinctive 9-mer 5′-ATG oriented oligonucleotide sequences has formed a 9-mer generic sense-codon-based DNA or and RNA oligonucleotide library accordingly. In one preferred embodiment, the complete collection of above 3,721 distinctive 9-mer 5′-ATG oriented sense oligonucleotide sequences has formed a 9-mer generic sense-codon-based oligonucleotide library. In one preferred embodiment, according to Watson-Crick DNA complementary rule, a corresponding 9-mer antisense 5′-CAT oriented generic antisense-codon-based oligonucleotide library has been produced and vice versa. In one other preferred embodiment, a series of Methionine oriented three-peptides, as expressed 9-mer 5′-ATG oriented oligonucleotides, have been produced either directly from mentioned sense oligonucleotides or indirectly from its corresponding antisense oligonucleotides and vice versa. Collection of all 400 distinctive Methionine oriented three-peptide sequences has formed a Methionine oriented three-peptide library, which becomes a specialized three-peptide library such as a peptide ingredient library.

Generic ORF Sense Oligonucleotide Libraries with 3′-Stop Codon Orientation

As discussed above, there are three major stop codons (5′-TAA, 5′-TGA, 5′-TAG). Only one stop codon is at 3′-end of a given ORF sequence. In a given ORF, For example, one stop codon (5′-TGA) at 3′ end occupies the first codon position which orients the entire ORF sequence from 3′ towards 5′ direction. The second codon position in succession of ORF sequence is occupied by one of the 61 codons. The third codon position in succession of ORF sequence is occupied by one of the 61 codons as well as each of the subsequent sequential codon positions in 3′ towards 5′ direction thereafter. The numbers of the distinctive 5′-TGA oriented ORF sequences increase with increasing length. The said numbers could be calculated as long as the specific length (n) and (m) were given according to algorithm of 61.sup.(n−m). In one embodiment, 9-mer 5′-TGA oriented ORF sequence is three-codon-length-long. 5′-TGA is selected one-codon-length-long sequence of orientation. Therefore, n=3, m=1, E=n−m. E is exponent. 61.sup.(3-1)=3,721. The total numbers of distinctive 9-mer 5′-TGA oriented ORF sequences are 3,721. The n^{th} codon occupies nucleotide positions (3n) to (3n-2) in 5′-TGA oriented n-codon-length long sequence. Each of nucleotide positions of the n^{th} codon in 5′-oriented triplet format is (3n), (3n−1) and (3n−2) respectively. Thus, the total numbers of distinctive 9-mer 5′-TGA oriented sequences are 3,721. The total numbers of distinctive 9-mer 5′-TAG oriented sequences are 3,721. The total numbers of distinctive 9-mer 5′-TAA oriented sequences are 3,721.

In one preferred embodiment, a collection of all the above distinctive 9-mer stop codon oriented sequences (3,721.times.3) has formed a generic 9-mer oligonucleotide library, which can be used for multiple purposes such as forming an ingredient or a probe library on a generic arrays. In another preferred embodiment, according to each said formula, each of said oligonucleotide library comprises substantially all of said oligonucleotides. In yet another preferred embodiment, according to each said formula, each of said oligonucleotide library consists essentially of said oligonucleotides. In one preferred embodiment, a collection of all 3,721 distinctive 9-mer 5′-TAG oriented ORF sequences has formed a generic 9-mer oligonucleotide library, which is capable to be used as a generic and all-purpose 9-mer oligonucleotide ingredient, probe and primer library. In another preferred embodiment, a collection of above 3,721 distinctive 9-mer 5′-TAG oriented oligonucleotide sequences has formed a 9-mer generic sense-codon-based DNA or and RNA oligonucleotide library accordingly. In one preferred embodiment, the complete collection of above 3,721 distinctive 9-mer 5′-TAG oriented sense oligonucleotide sequences has formed a 9-mer generic sense-codon-based oligonucleotide library. In one preferred embodiment, according to Watson-Crick DNA complementary rule, a corresponding 9-mer antisense 5′-CTA oriented generic antisense-codon-based oligonucleotide library has been produced and vice versa. In one other preferred embodiment, in accordance with Central Dogma, a series of Methionine oriented three-peptides, as expressed 9-mer 5′-TAG oriented oligonucleotides, have been produced either directly from mentioned sense oligonucleotides or indirectly from its corresponding antisense oligonucleotides and vice versa. Collection of all 400 distinctive Methionine oriented three-peptide sequences has formed a Methionine oriented three-peptide library, which becomes a specialized three-peptide library such as a peptide ingredient library.

Generic ORF Sense Oligonucleotide Libraries with Two-Codon-Restriction-Endonuclease-Recognition Sequence Orientations

The restriction-endonuclease-recognition sequence of two-codon is selected from the group of restriction endonucleases, without limiting the generality of the foregoing, which exclude any and all stop codons within the recognition sequence comprising: Aat II, Acc65 I, Acl I, Afe I, Afl II, Age I, Apa I, ApaL I, Ase I, Avr II, BamHl, BfrBl, Bgl II, Bme1580 I, BmgB I, BseY I, Btr I, BsiW I, BspD I, BspE I, BsrB I, BsrG I, BssH II, BssS I, Bst B I, BstZ17 I, Cla I, Dra I, Eag I, EcoR I, EcoR V, Fsp I, Hind III, Hpa I, Kas I, Kpn I, Mfe I, Mlu I, Msc I, Nae I, Nar I, Nco I, Nde I, NgoM IV, Nhe I, Nru I, Nsi I, PaeR7 I, Pci I, Pml I, PspOM I, Pst I, Pvu I Pvu II, Sac I, Sac II, Sal I, Sca I, Sfo I, Sma I, SnaB I, Spe I, Sph I, Ssp I, Stu I, Tli I, Xba I, Xho I, Xma I, Acc I, BsaW I, BsiHKA I, Bsp1286 I, MspA1 I, Sty I. The excluded restriction endonucleases with two-codon-recognition sequence are Bcl I, BspH I and Psi I.

(1) Generic ORF Sense Oligonucleotide Libraries with 5′-Two-Codon-Restriction-Endonuclease-Recognition Sequence Orientation

For example, 5′-GACGTC is two-codon-recognition sequence of Aat II. At each 5′ end of ORF sequence, 5′-GACGTC occupies the consecutive first and second codon positions that orient the entire ORF sequence from 5′ towards 3′ direction. The third codon position in succession of ORF sequence is occupied by one of the 61 codons. The fourth codon position in succession of ORF sequence is occupied by one of the 61 codons as well as each of subsequent sequential codon positions in 5′ towards 3′ direction thereafter. The numbers of the distinctive 5′-GACGTC oriented ORF sequences increase quantitatively to the length increasing. The said numbers could be calculated as long as the specific length (n) and (m) were given according to algorithm of 61.sup.(n−m). In one embodiment, 12-mer 5′-GACGTC oriented ORF sequence is four-codon-length-long. 5′-GACGTC is selected two-codon-length-long sequence of orientation. Therefore, n=4, m=2, E=n−m. E is exponent. 61.sup.(4-2)=3,721. The total numbers of distinctive 12-mer 5′-GACGTC oriented ORF sequences are 3,721. The n^{th} codon occupies nucleotide positions (3n-2) to (3n) in 5′-GACGTC oriented n-codon-length-long sequence. Each of nucleotide positions of the n^{th} codon in 5′-oriented triplet format is (3n−2), (3n−1) and (3n) respectively.

In one preferred embodiment, the collection of all 3,721 distinctive 12-mer 5′-GACGTC oriented ORF sequences have formed a generic 12-mer oligonucleotide library, which is capable to be used for all-purpose such as ingredient or probe or primer library. In another preferred embodiment, according to each said formula, each of said oligonucleotide library comprises substantially all of said oligonucleotides. In yet another preferred embodiment, according to each said formula, each of said oligonucleotide library consists essentially of said oligonucleotides.

In another preferred embodiment, a collection of above 3,721 distinctive 12-mer 5′-GACGTC oriented oligonucleotide sequences has formed a 12-mer generic sense-codon-based DNA or and RNA oligonucleotide library accordingly. In one preferred embodiment, the complete collection of above 3,721 distinctive 12-mer 5′-GACGTC oriented sense oligonucleotide sequences has formed a 12-mer generic sense-codon-based oligonucleotide library. In one preferred embodiment, according to Watson-Crick DNA complementary rule, a corresponding 12-mer antisense 5′-GACGTC oriented generic antisense-codon-based oligonucleotide library could be produced and vice versa. In one preferred embodiment, in accordance with Central Dogma, a series of NH₂-DV oriented four-peptides, as expressed 12-mer 5′-GACGTC oriented oligonucleotides, have been produced either directly from mentioned sense oligonucleotides or indirectly from its corresponding antisense oligonucleotides and vice versa. Collection of all 400 distinctive NH₂-DV oriented four-peptide sequences has formed a NH₂-DV oriented four-peptide library, which becomes a specialized four-peptide library such as an ingredient or antigen or episode library.

(2) Generic ORF Sense Oligonucleotide Libraries with 3′-Two-Codon-Restriction-Endonuclease-Recognition Sequence Orientation

For example, 5′-GACGTC is two-codon-recognition sequence of Aat II. At each 3′ end of ORF sequence, 5′-GACGTC occupies the consecutive first and second codon positions that orient the entire ORF sequence from 3′ towards 5′ direction. The third codon position in succession of ORF sequence is occupied by one of the 61 codons. The fourth codon position in succession of ORF sequence is occupied by one of the 61 codons as well as each of subsequent sequential codon positions in 3′ towards 5′ direction thereafter. The numbers of the distinctive 5′-GACGTC oriented ORF sequences increase with increasing length. The said numbers could be calculated as long as the specific length (n) and (m) were given according to algorithm of 61.sup.(n−m). In one embodiment, 12-mer 5′-GACGTC oriented ORF sequence is four-codon-length-long. 5′-GACGTC is selected two-codon-length-long sequence of orientation. Therefore, n=4, m=2, E=n−m. E is exponent. 61.sup.(4-2)=3,721. The total numbers of distinctive 12-mer 5′-GACGTC oriented ORF sequences are 3,721. The n^{th} codon occupies nucleotide positions (3n) to (3n-2) in 5′-GACGTC oriented n-codon-length long sequence. Each of nucleotide positions of the n^{th} codon in 5′-oriented triplet format is (3n), (3n−1) and (3n−2) respectively.

In one preferred embodiment, a collection of all the 3,721 distinctive 12-mer 5′-GACGTC oriented ORF sequences has formed a generic 12-mer oligonucleotide library, which is capable to be used for multiple purposes such as an ingredient or probe or primer library. In another preferred embodiment, 61 codons have replaced by 60 specific mammalian mitochondria codons. Collection of all 3,600 distinctive 12-mer 5′-GACGTC oriented mammalian mitochondria sequences has formed a 12-mer sense codon-based oligonucleotide library, which becomes a specialized 12-mer sense codon-based oligonucleotide of ingredient or probe or primer library for mammalian mitochondria. In yet another preferred embodiment, in accordance with Watson-Crick DNA complementary rule, 12-mer 5′-GACGTC oriented antisense-codon-based mammalian mitochondria oligonucleotide library was being produced precisely from its molecular mirror of 12-mer 5′-GACGTC oriented sense oligonucleotide library and vice versa.

Generic 5′-UTR and 3′-UTR Sense Oligonucleotide Libraries with Two-Codon-Restriction-Endonuclease-Recognition Sequence Orientations

The restriction-endonuclease-recognition sequence of two-codon is selected from the group of restriction endonucleases, without limiting the generality of the foregoing, which include any and all stop codons within the recognition sequence comprising: Aat II, Acc65 I, Acl I, Afe I, Afl II, Age I, Apa I, ApaL I, Ase I, Avr II, BamHl, BfrBl, Bgl II, Bme1580 I, BmgB I, BseY I, Btr I, BsiW I, BspD I, BspE I, BsrB I, BsrG I, BssH II, BssS I, Bst B I, BstZ17 I, Cla I, Dra I, Eag I, EcoR I, EcoR V, Fsp I, Hind III, Hpa I, Kas I, Kpn I, Mfe I, Mlu I, Msc I, Nae I, Nar I, Nco I, Nde I, NgoM IV, Nhe I, Nru I, Nsi I, PaeR7 I, Pci I, Pml I, PspOM I, Pst I, Pvu I Pvu II, Sac I, Sac II, Sal I, Sca I, Sfo I, Sma I, SnaB I, Spe I, Sph I, Ssp I, Stu I, Tli I, Xba I, Xho I, Xma I, Acc I, BsaW I, BsiHKA I, Bsp1286 I, MspA1 I, Sty I, Bcl I, BspH I and Psi I.

(1) Generic 5′-UTR and 3′-UTR Sense Oligonucleotide Libraries with 5′-Two-Codon-Restriction-Endonuclease-Recognition Sequence Orientation

For example, 5′-GACGTC is two-codon-recognition sequence of Aat II. At each 5′ end of non-coding sequence, 5′-GACGTC occupies the consecutive first and second codon positions that orient the entire non-coding sequence from 5′ towards 3′ direction. The third codon position in succession of non-coding sequence is occupied by one of the 64 codons. The fourth codon position in succession of non-coding sequence is occupied by one of the 64 codons as well as each of subsequent sequential codon positions in 5′ towards 3′ direction thereafter. The numbers of the distinctive 5′-GACGTC oriented non-coding sequences increase with increasing length. The said numbers could be calculated as long as the specific length (n) and (m) were given according to algorithm of 64.sup.(n−m). In one embodiment, 12-mer 5′-GACGTC oriented non-coding sequence is four-codon-length-long. 5′-GACGTC is selected two-codon-length-long sequence of orientation. Therefore, n=4, m=2, E=n−m. E is exponent. 64.sup.(n-2)=4,096. The total numbers of distinctive 12-mer 5′-GACGTC oriented non-coding sequences are 4,096. The n^{th} codon occupies nucleotide positions (3n−2) to (3n) in 5′-GACGTC oriented n-codon-length long sequence. Each of nucleotide positions of the n^{th} codon in 5′-oriented triplet format is (3n−2), (3n−1) and (3n) respectively.

In one preferred embodiment, a collection of all the 4,096 distinctive 12-mer 5′-GACGTC oriented non-coding sequences has formed a generic 12-mer oligonucleotide library, which is capable to be used for multiple purposes such as an ingredient or probe or primer library. In another preferred embodiment, according to each said formula, each of said oligonucleotide library comprises substantially all of said oligonucleotides. In yet another preferred embodiment, according to each said formula, each of said oligonucleotide library consists essentially of said oligonucleotides.

In another preferred embodiment, a collection of above 4,096 distinctive 12-mer 5′-GACGTC oriented oligonucleotide sequences has formed a 12-mer generic sense-codon-based DNA or and RNA oligonucleotide library accordingly. In one preferred embodiment, the complete collection of above 4,096 distinctive 12-mer 5′-GACGTC oriented sense oligonucleotide sequences has formed a 12-mer generic sense-codon-based oligonucleotide library. In one preferred embodiment, according to Watson-Crick DNA complementary rule, a corresponding antisense 12-mer 5′-GACGTC oriented generic antisense-codon-based oligonucleotide library has been produced and vice versa.

(2) Generic ORF Sense Oligonucleotide Libraries with 3′-Two-Codon-Restriction-Endonuclease-Recognition Sequence Orientation

For example, 5′-GACGTC is two-codon-recognition sequence of Aat II. At each 3′ end of non-coding sequence, 5′-GACGTC occupies the consecutive first and second codon positions that orient the entire non-coding sequence from 3′ towards 5′ direction. The third codon position in succession of non-coding sequence is occupied by one of the 64 codons. The fourth codon position in succession of non-coding sequence is occupied by one of the 64 codons as well as each of the subsequent sequential codon positions in 3′ towards 5′ direction thereafter. The numbers of the distinctive 5′-GACGTC oriented non-coding sequences increase with increasing length. The said numbers could be calculated as long as the specific length (n) and (m) were given according to algorithm of 64.sup.(n−m). In one embodiment, 12-mer 5′-GACGTC oriented non-coding sequence is four-codon-length-long. 5′-GACGTC is selected two-codon-length-long sequence of orientation. Therefore, n=4, m=2, E=n−m. E is exponent. 64.sup.(n-2)=4,096. The total numbers of distinctive 12-mer 5′-GACGTC oriented non-coding sequences are 4,096. The n^{th} codon occupies nucleotide positions (3n) to (3n−2) in 5′-GACGTC oriented n-codon-length long sequence. Each of nucleotide positions of the n^{th} codon in 5′-oriented triplet format is (3n), (3n−1) and (3n−2) respectively.

In one preferred embodiment, a collection of all the 4,096 distinctive 12-mer 5′-GACGTC oriented non-coding sequences has formed a generic 12-mer oligonucleotide library, which can be used as all-purpose of ingredient or probe or primer library. In another preferred embodiment, according to each said formula, each of said oligonucleotide library comprises substantially all of said oligonucleotides. In yet another preferred embodiment, according to each said formula, each of said oligonucleotide library consists essentially of said oligonucleotides.

In another preferred embodiment, a collection of above 4,096 distinctive 12-mer 5′-GACGTC oriented oligonucleotide sequences has formed a 12-mer generic sense-codon-based DNA or and RNA oligonucleotide library accordingly. In one preferred embodiment, the complete collection of above 4,096 distinctive 12-mer 5′-GACGTC oriented sense oligonucleotide sequences has formed a 12-mer generic sense-codon-based oligonucleotide library. In one preferred embodiment, according to Watson-Crick DNA complementary rule, a corresponding 12-mer antisense 5′-GACGTC oriented generic antisense-codon-based oligonucleotide library has been produced and vice versa.

### Generic ORF Sense Oligonucleotide Libraries

### (1) 5′-Generic ORF Sense Oligonucleotide Libraries

At each 5′ end of ORF sequence, one of the 61 codons occupies the first codon position that orients the entire ORF sequence from 5′ towards 3′ direction. The second codon position in succession of ORF sequence is occupied by one of the 61 codons. The third codon position in succession of ORF sequence is occupied by one of the 61 codons as well as each of subsequent sequential codon positions in 5′ towards 3′ direction thereafter. The numbers of the distinctive 5′-one-codon oriented ORF sequences increase with increasing length. The said numbers could be calculated as long as the specific length (n) and (m) were given according to algorithm of 61.sup.(n−m). In one embodiment, 9-mer 5′-one-codon oriented ORF sequence is three-codon-length-long. 5′-one-codon is selected one-codon-length-long sequence of orientation. Therefore, n=3, m=1, E=n−m. E is exponent. 61.sup.(3-1)=3,721. The total numbers of distinctive 9-mer 5′-one-codon oriented ORF sequences are 226,981 (3,721.times.61). The n^{th} codon occupies nucleotide positions (3n-2) to (3n) in 5′-one-codon oriented n-codon-length-long sequence. Each of nucleotide positions of the n^{th} codon in 5′-oriented triplet format is (3n−2), (3n−1) and (3n) respectively.

In one preferred embodiment, a collection of all the 226,981 distinctive 9-mer one-codon oriented ORF sequences has formed a generic 9-mer oligonucleotide library, which can be used as a generic 9-mer oligonucleotide probe and primer library. In another preferred embodiment, according to each said formula, each of said oligonucleotide library comprises substantially all of said oligonucleotides. In yet another preferred embodiment, according to each said formula, each of said oligonucleotide library consists essentially of said oligonucleotides.

In another preferred embodiment, a collection of above 226,981 distinctive 9-mer one-codon oriented oligonucleotide sequences has formed a 9-mer generic sense-codon-based DNA or and RNA oligonucleotide library accordingly. In one preferred embodiment, the complete collection of above 226,981 distinctive 9-mer one-codon oriented sense oligonucleotide sequences has formed a 9-mer generic sense-codon-based oligonucleotide library. In one preferred embodiment, according to Watson-Crick DNA complementary rule, a corresponding 9-mer antisense one-codon oriented generic antisense-codon-based oligonucleotide library has been produced and vice versa. In one other preferred embodiment, in accordance with Central Dogma, a series of one amino acid oriented four-peptides, as expressed 9-mer one-codon oriented oligonucleotides, have been produced either directly from mentioned sense oligonucleotides or indirectly from its corresponding antisense oligonucleotides and vice versa.

### (2) 3′-Generic ORF Oligonucleotide Libraries

At each 3′ end of ORF sequence, one of the 61 codons occupies the first codon position that orients the entire ORF sequence from 3′ towards 5′ direction. The second codon position in succession of ORF sequence is occupied by one of the 61 codons. The third codon position in succession of ORF sequence is occupied by one of the 61 codons as well as each of subsequent sequential codon positions in 3′ towards 5′ direction thereafter. The numbers of the distinctive 3′-one-codon oriented ORF sequences increase with increasing length. The said numbers could be calculated as long as the specific length (n) and (m) were given according to algorithm of 61.sup.(n−m). In one embodiment, 9-mer 3′-one-codon oriented ORF sequence is three-codon-length-long. 3′-one-codon is selected one-codon-length-long sequence of orientation. Therefore, n=3, m=1, E=n−m. E is exponent. 61.sup.(3-1)=3,721. The total numbers of distinctive 9-mer 3′-one-codon oriented ORF sequences are 226,981 (3,721.times.61). The n^{th} codon occupies nucleotide positions (3n) to (3n-2) in 3′-one-codon oriented n-codon-length-long sequence. Each of nucleotide positions of the n^{th} codon in 3′-oriented triplet format is (3n−2), (3n−1) and (3n) respectively.

In one preferred embodiment, a collection of all the 226,981 distinctive 9-mer one-codon oriented ORF sequences has formed a generic 9-mer oligonucleotide library, which can be used as an oligonucleotide probe and primer library. In another preferred embodiment, according to each said formula, each of said oligonucleotide library comprises substantially all of said oligonucleotides. In yet another preferred embodiment, according to each said formula, each of said oligonucleotide library consists essentially of said oligonucleotides.

In another preferred embodiment, a collection of above 226,981 distinctive 9-mer one-codon oriented oligonucleotide sequences has formed a 9-mer generic sense-codon-based DNA or and RNA oligonucleotide library accordingly. In one preferred embodiment, the complete collection of above 226,981 distinctive 9-mer one-codon oriented sense oligonucleotide sequences has formed a 9-mer generic sense-codon-based oligonucleotide library. In one preferred embodiment, according to Watson-Crick DNA complementary rule, a corresponding 9-mer antisense one-codon oriented generic antisense-codon-based oligonucleotide library has been produced and vice versa. In one other preferred embodiment, in accordance with Central Dogma, a series of one amino acid oriented three-peptides, as expressed 9-mer one-codon oriented oligonucleotides, have been produced either directly from mentioned sense oligonucleotides or indirectly from its corresponding antisense oligonucleotides and vice versa.

### (3) Generic ORF Sense Hexamer Oligonucleotide Library

In one embodiment, two codons were selected from the group consisting of the 61 codons at each time. By adding all possible combinations of two codons from the 61 codons without any overlap and repetition, Generic ORF Sense Hexamer Oligonucleotide Library is synthesized. It comprises 3,721 distinct deoxyoligonucleotides or 3,721 distinct oligonucleotides. Each of the deoxyoligonucleotides or oligonucleotides is two-codon-length-long (3.times.2 nucleotides) with 5′ towards 3′ direction. Any and all of the stop codons is excluded. The algorithm for construction of Generic ORF Sense Hexamer Oligonucleotide Library is 61.sup.n which is under the conditions: n=2, 61.sup.2=3,721; each of the 61 codons occupies the first codon position at 5′-end; eventually, a collection of 3,721 distinct hexamer oligonucleotides forms an oligonucleotide library.

In one preferred embodiment, according to each said formula, each of said oligonucleotide library comprises substantially all of said oligonucleotides. In yet another preferred embodiment, according to each said formula, each of said oligonucleotide library consists essentially of said oligonucleotides. In one other preferred embodiment, in accordance with Central Dogma, a series of one amino acid oriented two-peptides, as expressed 6-mer one-codon oriented oligonucleotides, have been produced either directly from mentioned sense oligonucleotides or indirectly from its corresponding antisense oligonucleotides and vice versa. Collection of all 400 distinctive one amino acid oriented two-peptide sequences has formed a one amino acid oriented two-peptide library, which becomes a generic two-peptide library.

### Generic 3′-Start Codon Oriented 5′-UTR Sense Oligonucleotide Libraries

For example, one start codon, such as 5′-ATG is added at 3′ end of 5′-UTR, 5′-ATG occupies the first codon position that orients the entire 5′-UTR sequence from 3′ towards 5′ direction. The second codon position in succession of 5′-UTR sequence is occupied by one of the 64 codons. The third codon position in succession of 5′-UTR sequence is occupied by one of the 64 codons as well as each of subsequent sequential codon positions in 3′ towards 5′ direction thereafter. The numbers of the distinctive 5′-ATG oriented 5′-UTR sequences increase with increasing length. The said numbers could be calculated as long as the specific length (n) and (m) were given according to algorithm of 64.sup.(n−m). In one embodiment, 9-mer 5′-ATG oriented 5′-UTR sequence is three-codon-length-long. 5′-ATG is selected one-codon-length-long sequence of orientation. Therefore, n=3, m=1, E=n−m. E is exponent. 64.sup.(3-1)=4,096. The total numbers of distinctive 9-mer 5′-ATG oriented 5′-UTR sequences are 4,096. The negative sign in front of n only indicates that codon position is in 5′-UTR. Therefore, the comparison of the absolute value of n and m does not take the negative sign into consideration. Based on the said principle, when m<n<infinity, the codon position is (m-n); the n^{th} codon occupies 5′-ATG oriented 5′-UTR nucleotide positions 3(1-n) to 3(1-n)+2 in 3′- towards 5′-direction when n>m, m=1. According to the said principle, each of nucleotide positions of the n^{th} codon in 5′ oriented triplet formation is 3(1-n), 3(1-n)+1 and 3(1-n)+2 respectively when n>m, m=1.

In one preferred embodiment, a collection of all 4,096 distinctive 9-mer 5′-ATG oriented 5′-UTR sequences has formed a generic oligonucleotide library. In one preferred embodiment, according to each said formula, each of said oligonucleotide library comprises substantially all of said oligonucleotides. In yet another preferred embodiment, according to each said formula, each of said oligonucleotide library consists essentially of said oligonucleotides.

In another preferred embodiment, a collection of above 4,096 distinctive 9-mer 5′-ATG oriented 5′-UTR oligonucleotide sequences has formed a 9-mer generic sense-codon-based DNA or and RNA oligonucleotide library accordingly. In one preferred embodiment, the complete collection of above 4,096 distinctive 9-mer 5′-ATG oriented 5′-UTR sense oligonucleotide sequences has formed a 9-mer generic sense-codon-based oligonucleotide library. In one preferred embodiment, according to Watson-Crick DNA complementary rule, a corresponding 9-mer antisense one-codon oriented generic antisense-codon-based oligonucleotide library has been produced and vice versa.

### Generic 5′-Stop Codon Oriented 3′-UTR Oligonucleotide Libraries

As discussed above, there are three major stop codons (5′-TAA, 5′-TGA, 5′-TAG). For example, one stop codon, such as 5′-TGA is added at 5′ end of 3′-UTR, 5′-TGA occupies the first codon position that orients the entire 3′-UTR sequence from 5′ to 3′. The second codon position in succession of 3′-UTR sequence is occupied by one of the 64 codons. The third codon position in succession of 3′-UTR sequence is occupied by one of the 64 codons as well as each of subsequent sequential codon positions in 5′ towards 3′ direction thereafter. The numbers of the distinctive 5′-TGA oriented 3′-UTR sequences increase with increasing length. The said numbers could be calculated as long as the specific length (n) and (m) were given according to algorithm of 64.sup.(n−m). In one embodiment, 9-mer 5′-TGA oriented 3′-UTR sequence is three-codon-length-long. 5′-TGA is selected one-codon-length-long sequence of orientation. Therefore, n=3, m=1, E=n−m. E is exponent. 64.sup.(3-1)=4,096. The total numbers of distinctive 9-mer 5′-TGA oriented 3′-UTR sequences are 4,096. The n^{th} codon occupies nucleotide positions (3n−2) to (3n) of the 5′-TGA oriented 3′-UTR sequence of n-codon-length-long. Each of nucleotide positions of the n^{th} codon in 5′-oriented triplet format is (3n−2), (3n−1) and (3n) respectively.

In one preferred embodiment, a collection of all the distinctive 9-mer 5′-stop codon oriented 3′-UTR sequences (4,096.times. 3) has formed a 9-mer generic oligonucleotide library. In one preferred embodiment, according to each said formula, each of said oligonucleotide library comprises substantially all of said oligonucleotides. In yet another preferred embodiment, according to each said formula, each of said oligonucleotide library consists essentially of said oligonucleotides.

In another preferred embodiment, a collection of above 4,096 distinctive 9-mer 5′-TGA oriented 3′-UTR oligonucleotide sequences has formed a 9-mer generic sense-codon-based DNA or and RNA oligonucleotide library accordingly. In one preferred embodiment, the complete collection of above 4,096 distinctive 9-mer 5′-TGA oriented 3′-UTR sense oligonucleotide sequences has formed a 9-mer generic sense-codon-based oligonucleotide library. In one preferred embodiment, according to Watson-Crick DNA complementary rule, a corresponding 9-mer antisense one-codon oriented generic antisense-codon-based oligonucleotide library has been produced and vice versa.

### Generic Antisense Start Codon Oriented Antisense Libraries

Antisense oligonucleotides with their analogues and derivatives, such as LNA are designed to bind their complementary sequences of mRNA. The bindings often inhibit the expression of the target peptides and proteins. Its application has a wide spectrum from clinical therapy (Stein et al., Science 261: 1004-1012, 1993) to food processing industry (Bachem et al., Bio/Technol. 12: 1101-1105, 1994).

Another concern is the suitable targeting areas for antisense oligonucleotide. There are a number of typical targeting locations of genes for antisense design, such as the 5′-cap region, the translation initiation region and the termination region. 5′-ATG and downstream sequences are generally regarded as the more promising target locations for antisense inhibition.

For example, at each 3′-end of antisense ORF sequence, the first antisense codon position is solely occupied by antisense start codon, such as 3′-TAC in 3′ towards 5′ direction. The second antisense codon position adjacent to the 5′ end of the anti-sense start codon, such as 3′-TAC is occupied by one of 61 antisense codons in 3′ towards 5′ direction. The third antisense codon position in succession of antisense ORF sequence is occupied by one of the 61 antisense codons as well as each of subsequent sequential antisense codon positions in 3′ towards 5′ direction thereafter. The numbers of the distinctive 3′-TAC oriented antisense ORF sequences increase with increasing length. The said numbers could be calculated as long as the specific length (n) and (m) were given according to algorithm of 61.sup.(n−m). In one embodiment, 3′-TAC oriented antisense ORF sequence is three-antisense-codon-length-long. 3′-TAC is selected antisense-one-codon-length-long antisense sequence of orientation. Therefore, n=3, m=1, E=n−m. E is exponent. 61.sup.(3-1)=3,721. The total numbers of distinctive 9-mer 3′-TAC oriented antisense ORF sequences are 3,721.

In one preferred embodiment, a collection of all the 3,721 distinctive 9-mer 3′-TAC oriented antisense ORF sequences has formed a generic 9-mer antisense oligonucleotide library, which can be used as a standardized and all-purpose, universal 9-mer antisense oligonucleotide probe and primer library. In one preferred embodiment, according to each said formula, each of said antisense oligonucleotide library comprises substantially all of said antisense oligonucleotides. In yet another preferred embodiment, according to each said formula, each of said antisense oligonucleotide library consists essentially of said antisense oligonucleotides. In one preferred embodiment, according to Watson-Crick DNA complementary rule, a corresponding 9-mer sense 5′-ATG oriented generic sense-codon-based oligonucleotide library has been produced and vice versa. In one other preferred embodiment, in accordance with Central Dogma, a series of Methionine oriented three-peptides, as expressed 9-mer 5′-ATG oriented oligonucleotides, have been produced either directly from mentioned corresponding sense oligonucleotides or indirectly from its corresponding antisense oligonucleotides and vice versa. Collection of all 400 distinctive Methionine oriented three-peptide sequences has formed a Methionine oriented three-peptide library, which becomes a specialized three-peptide library such as a peptide ingredient library.

Generic Peptide Libraries with N-terminal Orientation

For example, Methionine or Formylmethionine occupies the first amino acid position of peptide of N-terminal. The second amino acid position immediately adjacent to Methionine or Formylmethionine is occupied by one of the 20 Essential Amino Acids (EAA) in N-terminal towards C-terminal direction. The third amino acid position in succession of the peptide sequence is occupied by one of the 20 EAA as well as each of subsequent sequential amino acid positions in N-terminal towards C-terminal direction thereafter. The numbers of the distinctive Methionine or Formylmethionine oriented peptide increase with increasing length. The said numbers could be calculated as long as the specific length (n) and (m) were given according to the algorithm of 20.sup.(n−m). In one embodiment, Methionine selected one-amino-acid-length-long oriented sequence. Therefore, n=6, m=1, E=n−m. E is exponent. 20.sup.(6-1)=3,200,000. The total numbers of distinctive Methionine oriented 6-peptide sequences are 3,200,000.

In one preferred embodiment, according to each said formula, each of said peptide library comprises substantially all of said peptides. In yet another preferred embodiment, according to each said formula, each of said peptide library consists essentially of said peptides. In one preferred embodiment, a collection of all 3,200,000 distinctive Methionine oriented 6-peptide sequences has formed a generic 6-peptide library, which is capable to be used as a standardized, universal and all-purpose 6-peptide ingredient or antigen or epitope library. In one other preferred embodiment, in accordance with Central Dogma, 3,721 distinctive 9-mer 5′-ATG oriented oligonucleotides have been produced from the corresponding 400 distinctive Methionine oriented three-peptide sequences. Collection of all 3,721 distinctive 9-mer 5′-ATG oriented oligonucleotide sequences has formed a 9-mer 5′-ATG oriented oligonucleotide library, which becomes a specialized 9-mer oligonucleotide library such as, an oligonucleotide ingredient or probe or primer library. In one another preferred embodiment, in accordance with Watson-Crick DNA complementary rule, a 9-mer 5′-CAT oriented antisense oligonucleotide library was being produced precisely from its molecular mirror of 9-mer 5′-ATG oriented sense oligonucleotide library and vice versa.

Generic Peptide Libraries with C-terminal Orientation

As discussed above, one stop codon is at 3′-end of ORF sequence wherein peptide is released during protein synthesis. For example, the first amino acid position of C-terminal of peptide or protein may be occupied by one of the 20 EAA in C-terminal towards N-terminal direction. The second amino acid position in succession of C-terminal oriented peptide sequence is occupied by one of the 20 EAA in C-terminal towards N-terminal direction. The third amino acid position in succession of the peptide sequence is occupied by one of the 20 EAA as well as each of subsequent sequential amino acid positions in C-terminal towards N-terminal direction thereafter. The numbers of the distinctive C-terminal oriented peptide increase with increasing length. The said numbers could be calculated as long as the specific length (n) and (m) were given according to algorithm of 20.sup.(n−m). In one embodiment, One of the 20 EAA oriented 6-peptide sequence is six amino acids length long. One of the 20 EAA is selected one-amino-acid-length-long oriented sequence. Therefore, n=6, m=1, E=n−m. E is exponent. 20.sup.(6-1)=3,200,000. The total number of distinctive C-terminal oriented 6-peptide sequences is 64,000,000 (3,200,000.times.20). In one preferred embodiment, according to each said formula, each of said peptide library comprises substantially all of said peptides. In yet another preferred embodiment, according to each said formula, each of said peptide library consists essentially of said peptides. In one preferred embodiment, a collection of all 64,000,000 distinctive C-terminal oriented 6-peptide sequences has formed a generic 6-peptide library, which can be used as a standardized, universal and all-purpose 6-peptide ingredient or antigen or epitope library.

### Generic Peptide Libraries

### (1) Generic Peptide Libraries Between N-Terminal and C-Terminal of N-Terminal Orientation

For example, the first amino acid position at N-terminal is occupied by one of the 20 EAA. The second amino acid position immediately adjacent to the first amino acid position is occupied by one of the 20 EAA in N-terminal towards C-terminal direction. The third amino acid position in succession of the peptide sequence is occupied by one of the 20 EAA as well as each of subsequent sequential amino acid positions in N-terminal towards C-terminal direction thereafter. Therefore, the n^{th} amino acid position is occupied by one of the 20 essential amino acids in N-terminal towards C-terminal direction within a peptide sequence of n amino acids long. There are total 20.sup.(n-1).times.20 or 20.sup.(n−m).times.20 distinct n-peptide-length-long peptide of N-terminal oriented sequences. The numbers of the distinctive N-terminal oriented peptide increase with increasing length. The said numbers could be calculated as long as the specific length (n) and (m) were given according to algorithm of 20.sup.(n−m). In one embodiment, when n=6, m=1, E=n−m, 20.sup.(6-1)=3,200,000. The total number of distinctive N-terminal oriented 6-peptide sequences is 64,000,000 (3,200,000.times. 20).

In one preferred embodiment, according to each said formula, each of said peptide library comprises substantially all of said peptides. In yet another preferred embodiment, according to each said formula, each of said peptide library consists essentially of said peptides. In one preferred embodiment, a collection of all above 64,000,000 distinctive N-terminal oriented 6-peptide sequences has formed a generic 6-peptide library, which can be used as a standardized, universal and all-purpose 6-peptide ingredient or antigen or epitope library.

### (2) Generic Peptide Libraries Between N-Terminal and C-Terminal of C-Terminal Orientation

For example, the first amino acid position at C-terminal is occupied by one of the 20 EAA. The second amino acid position immediately adjacent to the first amino acid position is occupied by one of the 20 EAA in C-terminal towards N-terminal direction. The third amino acid position in succession of the peptide sequence is occupied by one of the 20 EAA as well as each of subsequent sequential amino acid positions in C-terminal towards N-terminal direction thereafter. Therefore, the n^{th} amino acid position is occupied by one of the 20 EAA in C-terminal towards N-terminal direction within a peptide sequence of n amino acids long. There are total 20.sup.(n−m).times.20 distinct n-amino-acid-length-long peptides of C-terminal oriented sequences. The numbers of the distinctive C-terminal oriented peptide increases with increasing length. The said numbers could be calculated as long as the specific length (n) and (m) were given according to algorithm of 20.sup.(n−m). In one embodiment, when n=6, m=1, E=n−m, 20.sup.(6-1)=3,200,000. The total number of distinctive C-terminal oriented 6-peptide sequences is 64,000,000 (3,200,000.times.20).

In one preferred embodiment, according to each said formula, each of said peptide library comprises substantially all of said peptides. In yet another preferred embodiment, according to each said formula, each of said peptide library consists essentially of said peptides. In one preferred embodiment, a collection of all above 64,000,000 distinctive C-terminal oriented 6-peptide sequences has formed a standardized universal 6-peptide library, which can be used as a standardized, universal and all-purpose 6-peptide antigen or epitope library.

Generic Peptide Libraries with Two-Amino-Acid of Restriction-Endonuclease-Recognition Sequence Orientations

The restriction endonuclease is selected from the group of restriction endonucleases, which have two-codon-recognition sequences that excluded any and all stop codons within the two codons. Examples of suitable restriction endonucleases include but are by no means limited to Aat II, Acc65 I, Acl I, Afe I, Afl II, Age I, Apa I, ApaL I, Ase I, Avr II, BamHl, BfrBl, Bgl II, Bme1580 I, BmgB I, BseY I, Btr I, BsiW I, BspD I, BspE I, BsrB I, BsrG I, BssH II, BssS I, Bst B I, BstZ17 I, Cla I, Dra I, Eag I, EcoR I, EcoR V, Fsp I, Hind III, Hpa I, Kas I, Kpn I, Mfe I, Mlu I, Msc I, Nae I, Nar I, Nco I, Nde I, NgoM IV, Nhe I, Nru I, Nsi I, PaeR7 I, Pci I, Pml I, PspOM I, Pst I, Pvu I Pvu II, Sac I, Sac II, Sal I, Sca I, Sfo I, Sma I, SnaB I, Spe I, Sph I, Ssp I, Stu I, Tli I, Xba I, Xho I, Xma I, Acc I, BsaW I, BsiHKA I, Bsp1286 I, MspA1 I, and Sty I. The excluded restriction endonucleases with two-codon recognition sequence are Bcl I, BspH I and Psi I. In one embodiment, the preferred panel of peptides comprising two amino acids deduced from the above restriction endonuclease recognition sequences.

(1) Generic Peptide Libraries with Two-Amino-Acid of Restriction-Endonuclease-Recognition Sequence of N-Terminal Orientation

For example, 5′-GACGTC is two-codon-recognition sequence of restriction endonuclease Aat II. NH₂-DV is encoded by 5′-GACGTC. In some embodiments, a two-amino-acid peptide from a restriction-endonuclease-recognition sequence is placed at N-terminal of a peptide. For example, NH₂-DV is placed at the consecutive first and second amino acids' positions of N-terminal of peptide, which orients the entire peptide sequence from N-terminal towards C-terminal. The consecutive first and second amino acid positions of peptide are solely occupied by selected two-amino-acid of two-codon-restriction-endonuclease-recognition sequence of, e.g. NH₂-DV in N-terminal towards C-terminal direction. The third amino acid position adjacent to C-terminal of NH₂-DV (the first and second amino acids' positions) is occupied by one of the 20 EAA in N-terminal towards C-terminal orientation. The fourth amino acid position in succession of peptide sequence is occupied by one of the 20 EAA as well as each of subsequent sequential amino acid positions in N-terminal towards C-terminal direction thereafter. Therefore, the nth amino acid position of peptide is occupied by one of 20.sup.(n-2) or 20.sup.(Erers) amino acids in N-terminal towards C-terminal orientated manner within n-peptide-length-long sequences. Erers means Exponent of restriction-endonuclease-recognition sequence. Erers is exponent. NH₂-DV is selected two-amino-acid-length-long sequence of orientation. In one embodiment, when n=6, m=2, Erers=n−m, 20.sup.(n-2)=160,000.

In one preferred embodiment, according to each said formula, each of said peptide library comprises substantially all of said peptides. In yet another preferred embodiment, according to each said formula, each of said peptide library consists essentially of said peptides. In one preferred embodiment, a collection of all the above 160,000 distinctive NH₂-DV oriented 6-peptide sequences has formed a standardized universal 6-peptide library, which can be used as a standardized, universal and all-purpose 6-peptide ingrediant or antigen or epitope library.

In one other preferred embodiment, in accordance with Central Dogma, 3,721 distinctive 12-mer 5′-GACGTC oriented oligonucleotides have been produced from the corresponding 400 distinctive NH₂-DV oriented four-peptide sequences. Collection of all 3,721 distinctive 12-mer 5′-GACGTC oriented oligonucleotide sequences has formed a 12-mer 5′-GACGTC oriented oligonucleotide library, which becomes a specialized 12-mer oligonucleotide library such as, an oligonucleotide ingredient or probe or primer library. In one another preferred embodiment, in accordance with Watson-Crick DNA complementary rule, a 12-mer antisense 5′-GACGTC oriented antisense oligonucleotide library was being produced precisely from its molecular mirror of 9-mer sense 5′-GACGTC oriented sense oligonucleotide library and vice versa.

(2) Generic Peptide Libraries with Two-Amino-Acid of Restriction-Endonuclease-Recognition Sequence of C-Terminal Orientation

Similarly, 5′-GACGTC is two-codon-recognition sequence of restriction endonuclease Aat II. DV-COOH is encoded by 5′-GACGTC. In one embodiment, a two-amino-acid peptide from a restriction-endonuclease-recognition sequence is placed at C-terminal of peptide. For example, DV-COOH is placed at the consecutive first and second amino acids positions of C-terminal of peptide, which orients the entire peptide sequence from C-terminal towards N-terminal direction. The consecutive first and second amino acid positions' of peptide is solely occupied by selected two-amino-acid of the two-codon-restriction-endonuclease-recognition sequence, e.g. DV-COOH in C-terminal towards N-terminal direction. The third amino acid position adjacent to N-terminal of DV-COOH (the first and second amino acids' positions) is occupied by one of the 20 EAA in C-terminal towards N-terminal direction. The fourth amino acid position in succession of peptide sequence is occupied by one of the 20 EAA as well as each of subsequent sequential amino acid positions in C-terminal towards N-terminal direction thereafter. Therefore, the nth amino acid position of peptide is occupied by one of 20.sup.(n-2) or 20.sup.(Erers) amino acids in C-terminal towards N-terminal orientated manner within n-peptide-length-long sequences. Erers means Exponent of restriction-endonuclease-recognition sequence. Erers is exponent. DV-COOH is selected two amino acids length long sequence of orientation. In another embodiment, when n=6, m=2, Erers=n−m, 20.sup.(n-2)=160,000.

In one preferred embodiment, according to each said formula, each of said peptide library comprises substantially all of said peptides. In yet another preferred embodiment, according to each said formula, each of said peptide library consists essentially of said peptides. In another preferred embodiment, a collection of all the above 160,000 distinctive DV-COOH oriented 6-peptide sequences has formed a standardized universal 6-peptide library, which can be used as a standardized, universal and all-purpose 6-peptide antigen or epitope library.

### GC Identical Oligonucleotide Panels

Poisson distribution of GC content reflects the GC contents of those oligonucleotide libraries which have been constructed according to algorithm of 61.sup.(n−m), wherein m=1.

In one embodiment, 3,721 distinctive 9-mer 5′-ATG oriented oligonucleotides of a library have been classified into seven GC Identical Panels according to GC content as following: (1) 64 distinctive oligonucleotides of 77.8% GC content, (2) 384 distinctive oligonucleotides of 66.7% GC content, (3) 928 distinctive oligonucleotides of 55.6% GC content, (4) 1,168 distinctive oligonucleotides of 44.4% GC content, (5) 820 distinctive oligonucleotides of 33.3% GC content, (6) 308 distinctive oligonucleotides of 22.2% GC content and (7) 49 distinctive oligonucleotides of 11.1% GC content. Each of the said panels includes all necessary and suitable positive and negative controls known in the art.

In another embodiment, 3,721 distinctive 9-mer 5′-TGA oriented oligonucleotides of a library have been classified into seven GC Identical Panels according to GC content as following: (1) 64 distinctive oligonucleotides with 77.8% GC content, (2) 384 distinctive oligonucleotides with 66.7% GC content, (3) 928 distinctive oligonucleotides with 55.6% GC content, (4) 1,168 distinctive oligonucleotides with 44.4% GC content, (5) 820 distinctive oligonucleotides with 33.3% GC content, (6) 308 distinctive oligonucleotides with 22.2% GC content and (7) 49 distinctive oligonucleotides with 11.1% GC content. Each of the said panels includes all necessary and suitable positive and negative controls known in the art.

In an alternative embodiment, 3,721 distinctive 9-mer 5′-TAG oriented oligonucleotides of a library have been classified into seven GC Identical Panels according to GC content as following: (1) 64 distinctive oligonucleotides with 77.8% GC content, (2) 384 distinctive oligonucleotides with 66.7% GC content, (3) 928 distinctive oligonucleotides with 55.6% GC content, (4) 1,168 distinctive oligonucleotides with 44.4% GC content, (5) 820 distinctive oligonucleotides with 33.3% GC content, (6) 308 distinctive oligonucleotides with 22.2% GC content and (7) 49 distinctive oligonucleotides with 11.1% GC content. Each of the said panels includes all necessary and suitable positive and negative controls known in the art.

In one embodiment, 4,096 distinctive 9-mer 5′-ATG oriented oligonucleotides of a library have been classified into seven GC Identical Panels according to GC content as following: (1) 64 distinctive oligonucleotides with 77.8% GC content, (2) 384 distinctive oligonucleotides with 66.7% GC content, (3) 960 distinctive oligonucleotides with 55.6% GC content, (4) 1,280 distinctive oligonucleotides with 44.4% GC content, (5) 960 distinctive oligonucleotides with 33.3% GC content, (6) 384 distinctive oligonucleotides with 22.2% GC content and (7) 64 distinctive oligonucleotides with 11.1% GC content. Each of the said panels includes all necessary and suitable positive and negative controls known in the art.

In another embodiment, 4,096 distinctive 9-mer 5′-TGA oriented oligonucleotides of a library have been classified into seven GC Identical Panels according to GC content as following: (1) 64 distinctive oligonucleotides with 77.8% GC content, (2) 384 distinctive oligonucleotides with 66.7% GC content, (3) 960 distinctive oligonucleotides with 55.6% GC content, (4) 1,280 distinctive oligonucleotides with 44.4% GC content, (5) 960 distinctive oligonucleotides with 33.3% GC content, (6) 384 distinctive oligonucleotides with 22.2% GC content and (7) 64 distinctive oligonucleotides with 11.1% GC content. Each of the said panels includes all necessary and suitable positive and negative controls known in the art.

In another embodiment, 4,096 distinctive 9-mer 5′-TAG oriented oligonucleotides of a library have been classified into seven GC Identical Panels according to GC content as following: (1) 64 distinctive oligonucleotides with 77.8% GC content, (2) 384 distinctive oligonucleotides with 66.7% GC content, (3) 960 distinctive oligonucleotides with 55.6% GC content, (4) 1,280 distinctive oligonucleotides with 44.4% GC content, (5) 960 distinctive oligonucleotides with 33.3% GC content, (6) 384 distinctive oligonucleotides with 22.2% GC content and (7) 64 distinctive oligonucleotides with 11.1% GC content. Each of the said panels includes all necessary and suitable positive and negative controls known in the art.

In yet another embodiment, 4,096 distinctive 12-mer antisense 5′-GGATCC (BamH I) oriented antisense oligonucleotides of an antisense library have been classified into seven GC Identical Panels according to GC content as following: (1) 64 distinctive antisense oligonucleotides with 91.7% GC content, (2) 384 distinctive antisense oligonucleotides with 75% GC content, (3) 960 distinctive antisense oligonucleotides with 66.7% GC content, (4) 1,280 distinctive antisense oligonucleotides with 58.3% GC content, (5) 960 distinctive antisense oligonucleotides with 50% GC content, (6) 384 distinctive antisense oligonucleotides with 41.7% GC content and (7) 64 distinctive antisense oligonucleotides with 33.3% GC content (Table 2). Each of the said panels includes all necessary and suitable positive and negative controls known in the art.

In some embodiments, antisense oligonucleotides with 77.8% GC content or greater are grouped together while antisense oligonucleotides with 11.1% GC content or less are grouped together respectively.

In other embodiments, the antisense oligonucleotides within a library that have the identical length and identical orientation are grouped according to GC content, which may subsequently be regrouped into a sub-library or sub GC Identical Antisense Oligonucleotide Panels. Each of the said sub GC Identical Antisense Oligonucleotide Panels includes all necessary and suitable positive and negative controls known in the art.

In one embodiment, the oligonucleotides of a given GC Identical Panel or sub GC Identical Panel have been elongated by adding a codon consisting of three consecutive universal bases, wherein said universal bases are selected from the group comprising 5′-nitroindole-2′-deoxyriboside, 3-nitropyrrole, inosine, pypoxanthine and combinations thereof. The said codon is being covalently linked at 5′-end of each said oligonucleotides.

In one another embodiment, the antisense oligonucleotides of a given GC Identical Panel or sub GC Identical Panel have been elongated by adding an antisense codon consisting of three consecutive universal bases, wherein said universal bases are selected from the group comprising 5′-nitroindole-2′-deoxyriboside, 3-nitropyrrole, inosine, pypoxanthine and combinations thereof. The said antisense codon is being covalently linked at 3′-end of each said antisense oligonucleotides.

In one embodiment, the oligonucleotides of a given GC Identical Panel or sub GC Identical Panel have been elongated by adding a codon consisting of three consecutive universal bases, wherein said universal bases are selected from the group comprising 5′-nitroindole-2′-deoxyriboside, 3-nitropyrrole, inosine, pypoxanthine and combinatorial thereof. The said codon is being covalently linked at 3′-end of each said oligonucleotides.

In one another embodiment, the antisense oligonucleotides of a given GC Identical Panel or sub GC Identical Panel have been elongated by adding an antisense codon consisting of three consecutive universal bases, wherein said universal bases are selected from the group comprising 5′-nitroindole-2′-deoxyriboside, 3-nitropyrrole, inosine, pypoxanthine and combinatorial thereof. The said antisense codon is being covalently linked at 5′-end of each said antisense oligonucleotides.

In one embodiment, each of the oligonucleotides of a given GC Identical Panel or sub GC Identical Panel has been incorporated with at least one LNA. Tm has been increased by about 2° C. degrees per each incorporated LNA, such as 2′-O, 4′-methylene-beta-D-robofuranosyl nucleotide monomer.

In one another embodiment, each of the antisense oligonucleotides of a given GC Identical Panel or sub GC Identical Panel has been incorporated with at least one LNA. Tm has been increased by about 2° C. degrees per each incorporated LNA, such as 2′-O, 4′-methylene-beta-D-robofuranosyl nucleotide monomer.

In one preferred embodiment, each of the 820 distinctive 9-mer 5′-ATG oriented sense oligonucleotides of a GC Identical Panel, wherein the said GC Identical Panel has 33.3% GC content, contains eight 2′-O, 4′-methylene-beta-D-robofuranosyl nucleotide monomer(s) within its 9-mer sense sequence. After the incorporation of LNA, Tm of each said sense oligonucleotide has been adjusted from 28° C. degrees to 42° C. degrees for both PCR and hybridization.

In one preferred embodiment, oligonucleotides or antisense oligonucleotides with at least one or more of LNA of a given GC Identical Panel or sub GC Identical Panel have been elongated by adding a codon consisting of three consecutive universal bases, wherein said universal bases are selected from the group comprising 5′-nitroindole-2′-deoxyriboside, 3-nitropyrrole, inosine, pypoxanthine and combinations thereof. The said codon is being covalently linked at 5′-end of each of the said oligonucleotides or antisense oligonucleotides

In another preferred embodiment, oligonucleotides or antisense oligonucleotides with at least two or more of LNA of a given GC Identical Panel or sub GC Identical Panel have been elongated by adding a codon consisting of three consecutive universal bases, wherein said universal bases are selected from the group comprising 5′-nitroindole-2′-deoxyriboside, 3-nitropyrrole, inosine, pypoxanthine and combinations thereof. The said codon is covalently linked at 3′-end of each of the said oligonucleotides or antisense oligonucleotides.

Each of the said oligonucleotides or antisense oligonucleotides of identical GC content is immobilized or linked or associate or attached or integrated to a carrier for delivery such as Lentiviruses, Adenoviruses, lipidoids, amphoteric liposomes, nanoparticles such as chitosan nanoparticles and other suitable carriers for antisense oligonucleotide delivery known in the art. In a set of each said oligonucleotide or antisense oligonucleotide, the said set comprising at least two copies of the said oligonucleotide or antisense oligonucleotide. The said oligonucleotide or antisense oligonucleotide comprises at least two said sets. The panels may be used alone or in combination. The said oligonucleotide or antisense oligonucleotide panels comprise substantially all of said oligonucleotides or antisense oligonucleotide. According to each said formula, each of said oligonucleotide panels consists essentially of said oligonucleotides or antisense oligonucleotides. The entire panel or individual oligonucleotide or antisense oligonucleotide thereof may be in a substantially aqueous phase. The Tm is being adjusted precisely according to the corresponding GC content or the numbers of incorporated LNA or both. In one preferred embodiment, each of the said oligonucleotides or antisense oligonucleotides which have the identical length and GC content interact with their targeting sequences either on a surface of a carrier or in aqueous phase under identical hybridization conditions determined by the calculation of Tm.

In one embodiment, a GC Identical Panel comprises substantially all of the oligonucleotides of one of the above-described formulae.

In one embodiment, a GC Identical Panel comprises substantially all of the antisense oligonucleotides of one of the above-described formulae.

In other embodiment, a GC Identical Panel comprises essentially of the oligonucleotides of one of the above-described formulae.

In other embodiment, a GC Identical Panel comprises essentially of the antisense oligonucleotides of one of the above-described formulae.

In another embodiment, each oligonucleotide of a GC Identical Panel consists substantially all of an oligonucleotide according to the specific formula for the respective panel.

In another embodiment, each antisense oligonucleotide of a GC Identical Panel consists substantially all of an antisense oligonucleotide according to the specific formula for the respective panel.

In one another embodiment, each oligonucleotide of a GC Identical Panel consists essentially of an oligonucleotide according to the specific formula for the respective panel.

In one another embodiment, each antisense oligonucleotide of a GC Identical Panel consists essentially of an antisense oligonucleotide according to the specific formula for the respective panel.

As will be appreciated by one of skilled in the art, a given single panel may consist of 2 or more sets of oligonucleotides or antisense oligonucleotides of one of the above-described formulae; 5 or more sets of oligonucleotides or of peptides of one of the above-described formulae; 10 or more sets of oligonucleotides or of peptides of one of the above-described formulae; 15 or more sets of oligonucleotides or antisense oligonucleotides of one of the above-described formulae; 20 or more sets of oligonucleotides or antisense oligonucleotides of one of the above-described formulae; 25 or more sets of oligonucleotides or antisense oligonucleotides of one of the above-described formulae; or 50 or more sets of oligonucleotides antisense oligonucleotides of one of the above-described formulae; 100 or more sets of oligonucleotides or antisense oligonucleotides of one of the above-described formulae; or 200 or more sets of oligonucleotides or antisense oligonucleotides of one of the above-described formulae; 300 or more sets of oligonucleotides or antisense oligonucleotides of one of the above-described formulae; or 500 or more sets of oligonucleotides or antisense oligonucleotides of one of the above-described formulae; 1,000 or more sets of oligonucleotides or antisense oligonucleotides of one of the above-described formulae; or 2,000 or more sets of oligonucleotides or antisense oligonucleotides of one of the above-described formulae; 3,000 or more sets of oligonucleotides or antisense oligonucleotides of one of the above-described formulae; or 5,000 or more sets of oligonucleotides or antisense oligonucleotides of one of the above-described formulae; 10,000 or more sets of oligonucleotides or antisense oligonucleotides of one of the above-described formulae; or 20,000 or more sets of oligonucleotides or antisense oligonucleotides of one of the above-described formulae; 50,000 or more sets of oligonucleotides or antisense oligonucleotides of one of the above-described formulae; or 100,000 or more sets of oligonucleotides or antisense oligonucleotides of one of the above-described formulae; 200,000 or more sets of oligonucleotides or antisense oligonucleotides of one of the above-described formulae; or 500,000 or more sets of oligonucleotides or antisense oligonucleotides of one of the above-described formulae.

### Synthesis of Oligonucleotide and Peptide

In one preferred embodiment, synthesis of oligonucleotides was carried out by phoshoramidite methods, such as Caruthers et al., Nucleic Acids Res. Symp. Ser. 7: 215-223, 1980; Beaucage et al., Tetrahedron Lett. 22: 1859-1862, 1981; McBride et al., Tetrahedron Lett. 24: 245-248, 1983; and Beaucage et al., Tetrahedron Lett. 48: 2223-2311, 1992; all of which are incorporated herein by reference in their entirety for all purposes.

In one preferred embodiment, the synthesis of oligonucleotides was processed by the H-phoshonate methods, such as Garegg et al., Chem. Scripta 25: 280-282, 1985; Garegg et al., Chem. Scripta 26: 59-62, 1986; Garegg et al., Tetrahedron Lett. 27: 4051-4054, 1986; Froehler et al., Nucleic Acids Res., 14: 5399-5407, 1986; Froehler et al., Tetrahedron Lett. 27: 469-4472, 1986; Froehler et al., Tetrahedron Lett. 27: 5575-5578, 1986; all of which are incorporated herein by reference in their entirety for all purposes.

In another preferred embodiment, the synthesis of oligonucleotides was carried out by an automated nucleic acid synthesizer which includes but is by no means limited to, ABI 381-A, ABI 391, ABI 392, ABI 3900 and Expedite® 8909 Nucleic Acid Synthesizer of PE Applied Biosystems® at a 0.2 μM scale using standard protocols in accordance with the manual of the manufacturer. Prior to the coupling step on a solid phase, the synthesized oligonucleotides then were purified, desalted and lyophilized at different grades of purity such as, PCR®-grade (ethanol precipitation to remove the salt), Probe-grade (purified by HPLC) and Gene-synthesis-grade (purified by polyacrylamide gel electrophoresis. The said purification methods and procedures are known to those of skilled in the art.

In one preferred embodiment, at specific defined discrete positions on a solid phase such as, a surface on silicon. In another preferred embodiment, the in-situ synthesis of oligonucleotides was carried out by photolithographic methods such as described by Fodor et al., Science 251: 767-773, 1991; Pease et al., Proc. Natl. Acad. Sci. U.S.A. 91: 5022-5026, 1994; Lockhart et al., Nat. Biotechol. 14: 1675, 1996; Pirrung et al., U.S. Pat. No. 5,143,854, 1992; Fodor et al., U.S. Pat. No. 5,445,934, 1995; Fodor et al., U.S. Pat. No. 5,510,270, 1996; Fodor et al., U.S. Pat. No. 5,800,992, 1998; all of which are incorporated herein by reference in their entirety for all purposes.

In another preferred embodiment, at specific defined discrete position on the surface of glass plate, in-situ synthesis of oligonucleotides was processed in accordance with methods as described by Southern et al., Genomic 13: 1008-1017, 1992; Maskos et al., Nucleic Acids Res. 20: 1679-1684, 1992; Southern et al., Nucleic Acids Res. 22: 1368-1373, 1994; all of which are incorporated herein by reference in their entirety for all purposes.

In another embodiment, in-situ synthesis of oligonucleotides and deposition on the perfluroinated hydrophobic surface of silicon dioxide was processed by Ink-jet printer heads as described by Blanchard et al., Biosensors & Bioelectronics 11: 687-690. This is incorporated herein by reference in its entirety for all purposes.

At the present time, the synthesis of oligonucleotides and peptides has become mature technology and standard laboratory operation procedures. It is the same for production of monoclonal antibodies. Moreover, many companies, such as Sigma-Genosys, Life Technologies and Washington Biotechnology Inc., provide routine service to produce the custom designed oligonucleotide, peptide and monoclonal antibodies tailored to different requirements and purposes. Those conditions allow one of ordinary skilled in the art to prepare oligonucleotides, peptides and monoclonal antibodies with undue experimentation.

### Analogues and Derivatives of Oligonucleotide and Peptide

Oligonucleotides or and antisense oligonucleotides deduced according to algorithm of 61.sup.(n−m) and 64.sup.(n−m) may contain restriction endonuclease recognition sequence(s) or promoter sequence(s) which include but are by no means limited to bacteriophage SP6, T3 and T7 sequence(s). The said oligonucleotides or and antisense oligonucleotides including both DNA and RNA oligonucleotides, which may have one or two or three or four or five or six universal base analogue(s) which include but are by no means limited to 5′-Nitroindole, 3-nitropyrrole, inosine and pypoxamthine. The said oligonucleotides may contain chemical modifications and substitutions on sugars, phosphates, phosphodiester bonds, bases, base analogues, universal bases and polyamide respectively or combinatorial. For example, the said chemical modifications and substitutions include but are by no means limited to 2′-O-alkylribose, 2′-O-Methylribonucleotide, Methylphosphonates, Morpholine, Phosphorothioate, Phosphordithioate, Sulfamate, H-phosphonate, phosphoroamidates, phosphotriesters, [(alpha)]-anomeric and the like. The said oligonucleotide analogues include but are by no means limited to Peptide Nucleic Acid (PNA), Locked Nucleic Acid (LNA), Locked Nucleic Acid (LNA), Peptide Nucleic Acid (PNA), Morpholino phosphoroamidate (MF), 2′-O-Methoxyethyl oligonucleotide(s) (2′-MOE), 2′-O-Methyl (2′-OME), Phosphoroamidate, Methylphosphonate and Universal base. The said oligonucleotides analogues include the modified nucleotide units, which possess energy emission patterns of a light emitting chemical compound or a quenching compound such as, hypoxanthine, mercaptopurine, selenopurine, 2-aminopurine, 2,4-diselenouracil and 2,4-dithiouracil. Additionally, the said modifications and substitutions include modifications and substitutions known or under development or to be developed to the extent that such alterations facilitate or have no negative affect when the said oligonucleotides or and antisense oligonucleotides hybridize to complementary targeting sequences in vitro or vivo. The said oligonucleotides or and antisense oligonucleotides may contain minor deletions, insertions and additions of codons or bases to the extent that such alterations facilitate or do not negatively affect when the said oligonucleotides or and antisense oligonucleotides hybridize complementary targeting sequences in vitro or vivo. The said oligonucleotides or and antisense oligonucleotides may be DNA, RNA, cDNA, mRNA, Anti-sense DNA, Anti-sense mRNA, DNA-RNA hybrid, and Peptide Nucleic Acids (PNA) in the format of either single strand or double strands. The said oligonucleotides or and antisense oligonucleotides may be labeled by a chemical composition(s), which produces specific detectable signal by radioactive ray, electromagnetic radiation, immunochemistry, biochemistry and photochemistry. Those labeling chemical composition include but are by no means limited to radioisotopes such as 3.sup.H, 14.sup.C, 32.sup.P, 33.sup.P, and 35.sup.S.; biotin; fluorescent molecules such as fluorescein isothiocyanate (FITC), Texas red, green fluorescent protein, rhodamines, tetramenthylrhodamine isothiocyanate (TRITC), 4, 4-difluoro-4-bora-3a, 4a-diaza-s-indacene, lissamine, 5′-carboxy-fluorescein, 2′,7′-dimethoxy-4′,5′-dichloro-6 carboxy-fluorescein, phycoerythrin, Cy2, Cy3, Cy3.5, Cy5, Cy5.5, Cy7; enzymes such as alkaline phosphates, horse radish peroxidase; substrates; nucleotide chromophores; chemiluminescent moieties; bioluminescent moieties; phosphorescent compounds, magnetic particles. The analogues and derivatives also include natural peptide, polypeptide and protein which contained the chemical modifications or and substitutions on amino acid and or on its analogous structures which deviates from and within the said peptide, polypeptide and protein sequences. The said chemical modifications on amino acid and on its analogous include but are by no means limited to hydroxylation, methylation, acetylation, carboxylation and phosphorylation. It also includes the addition of lipids and carbohydrate polymers to the side chains of amino acid residues of the said peptides, polypeptides and proteins.

One of the purposes of chemical modifications on those said oligonucleotides, particularly antisense oligonucleotides in the invention is to level nuclease resistance. The pharmacological effect of antisense oligonucleotides is depend on a number of aspects, which include but are by no means to be limited to the stability of the species in the presence of nucleases, penetration of cell membrane, reaching the targets and the fidelity of the hybridization. Those chemical modifications have taken many forms such as modification on sugar moiety, base ring and sugar-phosphate backbone.

In one preferred embodiment, the melting temperature of pre-synthesized oligonucleotides or and antisense oligonucleotides has been adjusted by incorporation of appropriate number of LNA monomer(s) in their sequences. In other preferred embodiment, Tm of pre-synthesized oligonucleotides or and antisense oligonucleotides has been adjusted to 40° C. by incorporation of an appropriate number of LNA monomer(s) in their sequences. In other preferred embodiments, Tm of pre-synthesized oligonucleotides or and antisense oligonucleotides has been adjusted between 40° C. to 50° C. under suitable hybridization conditions for oligonucleotides or and antisense oligonucleotides by incorporation of an appropriate number of LNA monomer(s) in their sequences. In one preferred embodiment, the incorporation of LNA and adjustment of pre-synthesized oligonucleotides or and antisense oligonucleotides have been performed according to the methods described by Beaucage et al., Tetrahedron Lett., 48(12) 2223-2311, 1992; Beaucage et al., Tetrahedron Lett., 49(28) 6123-6194, 1993; Imsnish et al., U.S. Pat. No. 6,268,490, 2001; Tolstrup et al., Nucleic Acids Res., 31: 3758-3762, 2003; all of which are incorporated herein by reference in their entirety for all purposes.

Overall, the methods of preparing, synthesizing, modification and application for both antisense and sense oligonucleotides include but are by no means to be limited to U.S. Pat. Nos. 7,495,088; 7,235,650; 7,138,517; 7,115,738; 7,037,646; 6,919,439; 6,900,301; 6,900,297; 6,828,434; 6,756,496; 6,653,458; 6,639,061; 6,537,973; 6,531584; 6,495,671; 6,399,754; 6,395,548; 6,339,066; 6,307,040; 6,271,357; 6,242,428; 6,214,551; 6,207,649; 6,200,960; 6,197,584; 6,121,433; 6,060,458; 6,025,482; 6,005,087; 5,977,083; 5,969,118; 5,965,721; 5,96,425; 5,939,402; 5,872,232; 5,859,221; 5,852,182; 5,808,027; 5,792,844; 5,783,682; 5,661,1345,637,573; 5,620,963; 5,618,704; 5,610,289; 5,607,923; 5,602,240; 5,599,797; 5,587,361; 5,576,302; 5,565,555; 5,541,307; 5,489,677; 5,386,023; 5,256,648; U.S. Pat. Appl. Nos. 20040014644; 20030045705; 20020155989; all of which are incorporated herein by reference in their entirety for all purposes.

### EXAMPLES

The following examples are intended to provide detailed illustrations of the present invention but are by no means limited to the invention thereof.

### Example 1

5′-End Start Codon Oriented Codon-Based Oligonucleotide Library Construction

A library with 5′-end start codon orientation was constructed. For example, a library of oligonucleotides consists of all possible combinations of 61 codons with a start codon, such as 5′-ATG, as 5′-end terminal codon for each oligonucleotide at a given length and a peptide library corresponding to amino acids sequences deduced from amino acid coding sequences. The length of the entire sequence (n) of each oligonucleotide including selected sequence of orientation (m) in within was measured by codon. n is an integer. m is an integer. n>m. m=1. 5′-ATG is selected sequence of orientation within the entire sequence of each oligonucleotide of the library. The length of selected sequence of orientation (m) was measured by codon or expressed codon. As will be appreciated by one of skilled in the art, the result of this arrangement is that the oligonucleotides will preferentially hybridize to regions of template strand (antisense) of genomic DNA, or 1^{st} single strand of cDNA upstream of and including an antisense start codon, such as 5′-CAT within the antisense coding region of antisense ORF in 5′ towards 3′ direction due to the fact that sequences corresponding to termination codons are specifically excluded. As will be appreciated by ordinary skilled in the art, in accordance with Watson-Crick DNA complementary rule, a corresponding antisense-codon-based antisense oligonucleotide library was being constructed as well and vice versa. In accordance with Central Dogma, a series of corresponding peptides, as expressed oligonucleotides, have been produced either directly from mentioned sense oligonucleotides or indirectly from its corresponding antisense oligonucleotides and vice versa.

### Example 2

3′-End Antisense Start Codon Oriented Antisense-Codon-Based Oligonucleotide Library Construction

A library with 3′-end antisense start codon orientation was constructed. For example, a library of antisense oligonucleotides consists of all possible combinations of 61 antisense amino acid coding codons with an antisense start codon, such as 5′-CAT, as 3′-end terminal antisense codon for each antisense oligonucleotide at a given length. 61 antisense amino acid coding codons are referred to 61 antisense codons hereafter. The length of the entire antisense sequence (n) of each antisense oligonucleotide including selected antisense sequence of orientation (m) in within was measured by antisense codon. n is an integer. m is an integer. n>m. m=1. 5′-CAT is selected antisense sequence of orientation of the entire antisense sequence within each antisense oligonucleotide of the library. The length of selected antisense sequence of orientation (m) was measured by antisense codon. As will be appreciated by one of skilled in the art, these antisense oligonucleotides will preferentially hybridize to regions of non-template strand (sense) of genomic DNA, or mRNA or 2^{nd} single strand of cDNA downstream of and including a start codon such as 5′-ATG within the coding region of ORF in 5′ towards 3′ direction due to the fact that antisense sequences corresponding to antisense termination codons are specifically excluded. As will be appreciated by ordinary skilled in the art, in accordance with Watson-Crick DNA complementary rule, a corresponding sense-codon-based oligonucleotide library was being constructed as well and vice versa. In accordance with Central Dogma, a series of corresponding peptides, as expressed oligonucleotides, have been produced either indirectly from mentioned antisense oligonucleotides or directly from its corresponding sense oligonucleotides and vice versa.

### Example 3

3′-End Antisense Start Codon Oriented Antisense-Codon-Based Mammalian Mitochondria Oligonucleotide Library Construction

A library with 3′-end antisense start codon orientation was constructed. For example, a library of mammalian mitochondria antisense oligonucleotides consists of all possible combinations of 60 mammalian mitochondria antisense amino acid coding codons with an antisense start codon, such as 5′-TAT, as 3′-end terminal antisense codon for each antisense mammalian mitochondria oligonucleotide at a given length. 60 mammalian mitochondria antisense amino acid coding codons are referred to 60 mammalian mitochondria antisense codons hereafter. The length of the entire antisense sequence (n) of each antisense oligonucleotide including selected antisense sequence of orientation (m) in within was measured by antisense codon. n is an integer. m is an integer. n>m. m=1. 5′-TAT is selected antisense sequence of orientation of the entire antisense sequence within each antisense oligonucleotide of the library. The length of selected antisense sequence of orientation (m) was measured by antisense codon. As will be appreciated by one of skilled in the art, these antisense oligonucleotides will preferentially hybridize to regions of non-template strand (sense) of mammalian mitochondria DNA, or mRNA or 2^{nd} single strand of cDNA downstream of and including a start codon such as 5′-ATA within the coding region in 5′ towards 3′ direction due to the fact that antisense sequences corresponding to antisense termination codons are specifically excluded. As will be appreciated by ordinary skilled in the art, in accordance with Watson-Crick DNA complementary rule, a corresponding mammalian mitochondria sense codon-based RNA oligonucleotide library was being constructed as well and vice versa. In accordance with Central Dogma, a series of corresponding peptides, as expressed oligonucleotides of mammalian mitochondria, have been produced either indirectly from mentioned antisense oligonucleotides or directly from its corresponding sense oligonucleotides and vice versa.

### Example 4

3′-End Stop Codon Oriented Codon-Based Oligonucleotide Library Construction

A library with 3′-end stop codon orientation was constructed. For example, a library of oligonucleotides consists of all possible combinations of 61 codons with a stop codon, such as 5′-TGA as 3′-end terminal codon for each oligonucleotide at a given length and a peptide library corresponding to amino acids sequences deduced from amino acid coding sequences excluding 3′-end stop codon. The length of the entire sequence (n) of each oligonucleotide including selected sequence of orientation (m) in within was measured by codon. n is an integer. m is an integer. n>m. m=1. 5′-TGA is selected sequence of orientation within the entire sequence of each oligonucleotide of the library. The length of selected sequence of orientation (m) was measured by codon or expressed codon. As will be appreciated by one of skilled in the art, the result of this arrangement is that the oligonucleotides will preferentially hybridize to regions of template strand (antisense) of genomic DNA, or 1^{st} single strand of cDNA downstream of and including an antisense stop codon such as 5′-TCA within the antisense coding region of antisense ORF in 5′ towards 3′ direction due to the fact that sequences corresponding to termination codons are specifically excluded. As will be appreciated by ordinary skilled in the art, in accordance with Watson-Crick DNA complementary rule, a corresponding antisense-codon-based RNA oligonucleotide library was being constructed as well and vice versa. In accordance with Central Dogma, a series of corresponding peptides, as expressed oligonucleotides, have been produced either directly from mentioned sense oligonucleotides or indirectly from its corresponding antisense oligonucleotides and vice versa. In accordance with Central Dogma, a series of corresponding peptides, as expressed oligonucleotides, have been produced either directly from mentioned sense oligonucleotides or indirectly from its corresponding antisense oligonucleotides and vice versa.

### Example 5

5′-End Antisense Stop Codon Oriented Antisense-Codon-Based Oligonucleotide Library Construction

A library with 5′-end antisense stop codon orientation was constructed. For example, a library of antisense oligonucleotides consists of all possible combinations of 61 antisense codons with an antisense stop codon, such as 5′-TCA, as 5′-end antisense terminal codon for each antisense oligonucleotide at a given length. The length of the entire antisense sequence (n) of each antisense oligonucleotide including selected antisense sequence of orientation (m) in within was measured by antisense codon. n is an integer. m is an integer. n>m. m=1. 5′-TCA is selected antisense sequence of orientation within the entire antisense sequence of each antisense oligonucleotide of the library. The length of selected antisense sequence of orientation (m) was measured by antisense codon. As will be appreciated by one of skilled in the art, these antisense oligonucleotides will preferentially hybridize to regions of non-template strand (sense) of genomic DNA, or mRNA or 2^{nd} single strand of cDNA upstream of and including a stop codon such as 5′-TGA within the coding region of ORF in 5′ towards 3′ direction due to the fact that antisense sequences corresponding to antisense termination codons are specifically excluded. As will be appreciated by ordinary skilled in the art, in accordance with Watson-Crick DNA complementary rule, a corresponding sense codon-based oligonucleotide library was being constructed as well and vice versa. In accordance with Central Dogma, a series of corresponding peptides, as expressed oligonucleotides, have been produced either indirectly from mentioned antisense oligonucleotides or directly from its corresponding sense oligonucleotides and vice versa.

### Example 6

5′-End Antisense Stop Codon Oriented Antisense-Codon-Based Mammalian Mitochondria Oligonucleotide Library Construction

A library with 5′-end antisense stop codon orientation was constructed. For example, a library of Mammalian Mitochondria antisense oligonucleotides consists of all possible combinations of 60 Mammalian Mitochondria antisense codons with an antisense stop codon, such as 5′-TCT, as 5′-end antisense terminal codon for each antisense oligonucleotide at a given length. The length of the entire antisense sequence (n) of each Mammalian Mitochondria antisense oligonucleotide including selected antisense sequence of orientation (m) in within was measured by antisense codon. n is an integer. m is an integer. n>m. m=1. 5′-TCT is selected antisense sequence of orientation within the entire antisense sequence of each Mammalian Mitochondria antisense oligonucleotide of the library. The length of selected antisense sequence of orientation (m) was measured by antisense codon. As will be appreciated by one of skilled in the art, these Mammalian Mitochondria antisense oligonucleotides will preferentially hybridize to regions of non-template strand (sense) of Mammalian Mitochondria DNA, or mRNA or 2^{nd} single strand of cDNA upstream of and including a stop codon such as 5′-AGA within the coding region in 5′ towards 3′ direction due to the fact that antisense sequences corresponding to antisense termination codons are specifically excluded. As will be appreciated by ordinary skilled in the art, in accordance with Watson-Crick DNA complementary rule, a corresponding mammalian mitochondria sense-codon-based oligonucleotide library was being constructed as well and vice versa. In accordance with Central Dogma, a series of corresponding peptides, as expressed oligonucleotides of mammalian mitochondria, have been produced either indirectly from mentioned antisense oligonucleotides or directly from its corresponding sense oligonucleotides and vice versa.

### Example 7

5′-End Two-Codon-Restriction-Enzyme-Recognition Sequence Oriented Codon-Based Oligonucleotide Library Construction

A library with orientations of two-codon-restriction-enzyme-recognition sequence at either 5′-end or 3′-end was constructed. For example, a library of oligonucleotides consists of all possible combinations of 61 codons with a two-codon-restriction-enzyme-recognition sequence, such as 5′-GACGTC (Aat II), as 5′-end terminal oriented two consecutive codons for each oligonucleotide at a given length and a peptide library corresponding to amino acids sequences deduced from amino acid coding sequences. The length of the entire sequence (n) of each oligonucleotide including selected sequence of orientation (m) in within was measured by codon. n is an integer. m is an integer. n>m. m=2. 5′-GACGTC (Aat II) is selected sequence of orientation within the entire sequence of each oligonucleotide of the library. The length of selected sequence of orientation (m) was measured by codon or expressed codon. As will be apparent to one of skilled in the art, the restriction-endonuclease-recognition sequences exclude termination codons within their recognition sequences in the library. The result of this arrangement is that the oligonucleotides will preferentially hybridize to regions of template strand (antisense) of genomic DNA, or 1^{st} single strand of cDNA upstream of and including an antisense-two-codon-restriction-endonuclease-recognition sequence, such as 5′-GACGTC (Aat II), within the antisense coding region of antisense ORF in 5′ towards 3′ direction due to the fact that sequences corresponding to termination codons are specifically excluded. As will be appreciated by ordinary skilled in the art, in accordance with Watson-Crick DNA complementary rule, a corresponding antisense-codon-based oligonucleotide library was being constructed as well and vice versa. In accordance with Central Dogma, a series of corresponding peptides, as expressed oligonucleotides, have been produced either directly from mentioned sense oligonucleotides or indirectly from its corresponding antisense oligonucleotides and vice versa.

### Example 8

3′-End Antisense-Two-Codon-Restriction-Enzyme-Recognition Sequence Oriented Antisense-Codon-Based Oligonucleotide Library Construction

A library with orientations of antisense-two-codon-restriction-endonuclease-recognition sequence at either 3′-end or 5′-end was constructed. For example, a library of antisense oligonucleotides consists of all possible combinations of 61 antisense codons with an antisense-two-codon-restriction-endonuclease-recognition sequence, such as 5′-GACGTC (Aat II), as 3′-end terminal two consecutive antisense codons for each antisense oligonucleotide at a given length. The length of the entire antisense sequence (n) of each antisense oligonucleotide including selected antisense sequence of orientation (m) in within was measured by antisense codon. n is an integer. m is an integer. n>m. m=2. 5′-GACGTC (Aat II) is selected antisense sequence of orientation within the entire antisense sequence of each antisense oligonucleotide of the library. The length of selected antisense sequence of orientation (m) was measured by antisense codon. As will be apparent to one of skilled in the art, antisense restriction endonuclease recognition sequences exclude antisense termination codons within their antisense recognition sequence in the library. The result of this arrangement is that these antisense oligonucleotides will preferentially hybridize to regions of non-template (sense) strand of genomic DNA, or mRNA or 2^{nd} single strand of cDNA downstream of and including a two-codon-restriction-enzyme-recognition sequence, such as 5′-GACGTC (Aat II), within the coding region of ORF in 5′ towards 3′ direction due to the fact that antisense sequences corresponding to antisense termination codons are specifically excluded. As will be appreciated by ordinary skilled in the art, in accordance with Watson-Crick DNA complementary rule, a corresponding sense-codon-based oligonucleotide library was being constructed as well and vice versa. In accordance with Central Dogma, a series of corresponding peptides, as expressed oligonucleotides, have been produced either indirectly from mentioned antisense oligonucleotides or directly from its corresponding sense oligonucleotides and vice versa.

### Example 9

3′-End Antisense-Two-Codon-Restriction-Enzyme-Recognition Sequence Oriented Mammalian Mitochondria Antisense-Codon-Based Oligonucleotide Library Construction

A library with orientations of either 3′-end antisense-two-codon-restriction-endonuclease-recognition sequence at either 3′-end or 5′-end was constructed. For example, a library of Mammalian Mitochondria antisense oligonucleotides consists of all possible combinations of 60 Mammalian Mitochondria antisense codons with an antisense-two-codon-restriction-endonuclease-recognition sequence, such as 5′-GACGTC (Aat II), as 3′-end terminal two consecutive antisense codons for each Mammalian Mitochondria antisense oligonucleotide at a given length. The length of the entire antisense sequence (n) of each Mammalian Mitochondria antisense oligonucleotide including selected antisense sequence of orientation (m) in within was measured by antisense codon. n is an integer. m is an integer. n>m. m=2. 5′-GACGTC (Aat II) is selected antisense sequence of orientation within the entire antisense sequence of each Mammalian Mitochondria antisense oligonucleotide of the library. The length of selected antisense sequence of orientation (m) was measured by antisense codon. As will be apparent to one of skilled in the art, antisense restriction endonuclease recognition sequences exclude Mammalian Mitochondria antisense termination codons within their antisense recognition sequence in the library. The result of this arrangement is that these Mammalian Mitochondria antisense oligonucleotides will preferentially hybridize to regions of non-template (sense) strand of Mammalian Mitochondria DNA, or mRNA or 2^{nd} single strand of Mammalian Mitochondria cDNA downstream of and including a two-codon-restriction-enzyme-recognition sequence, such as 5′-GACGTC (Aat II), within the coding region in 5′ towards 3′ direction due to the fact that antisense sequences corresponding to antisense termination codons are specifically excluded. As will be appreciated by ordinary skilled in the art, in accordance with Watson-Crick DNA complementary rule, a corresponding mammalian mitochondria sense-codon-based oligonucleotide library was being constructed as well and vice versa. In accordance with Central Dogma, a series of corresponding peptides, as expressed oligonucleotides of mammalian mitochondria, have been produced either indirectly from mentioned antisense oligonucleotides or directly from its corresponding sense oligonucleotides and vice versa.

### Example 10

5′-End Two-Codon-Restriction-Enzyme-Recognition Sequence Oriented Non-Coding Region Sense Oligonucleotide Library Construction

A library with orientations of two-codon-restriction-enzyme-recognition sequence at either 5′-end sequence or 3′-end was constructed. For example, a library of oligonucleotides consists of all possible combinations of 64 codons with a two-codon-restriction-enzyme-recognition sequence, such as 5′-TCATGA (BspH I), as 5′-end terminal oriented two consecutive codons for each oligonucleotide at a given length. The length of the entire sequence (n) of each oligonucleotide including selected sequence of orientation (m) in within was measured by codon. n is an integer. m is an integer. n>m. m=2. 5′-TCATGA (BspH I) is selected sequence of orientation within the entire sequence of each oligonucleotide of the library. The length of selected sequence of orientation (m) was measured by codon. As will be apparent to one of skilled in the art, the restriction endonuclease recognition sequences include termination codons within their recognition sequences are included in the library. The result of this arrangement is that the oligonucleotides will preferentially hybridize to regions of template strand (antisense) of genomic DNA, or 1^{st} single strand of cDNA upstream of and including an antisense-two-codon-restriction-endonuclease-recognition sequence, such as 5′-TCATGA (BspH I), within the antisense 5′-UTR or upstream of and including an antisense-two-codon-restriction-endonuclease-recognition sequence, such as 5′-TCATGA (BspH I), within the antisense 3′-UTR regions in 5′ towards 3′ direction due to the fact that sequences corresponding to termination codons are specifically included. As will be appreciated by ordinary skilled in the art, in accordance with Watson-Crick DNA complementary rule, a corresponding antisense-codon-based oligonucleotide library was being constructed as well and vice versa.

### Example 11

3′-End Antisense-Two-Codon-Restriction-Enzyme-Recognition Sequence Oriented Non-Coding Region Antisense Oligonucleotide Library Construction

A library with orientations of antisense-two-codon-restriction-endonuclease-recognition sequence at either 3′-end or 5′-end was constructed. For example, a library of antisense oligonucleotides consists of all possible combinations of 64 antisense codons with an antisense-two-codon-restriction-endonuclease-recognition sequence, such as 5′-TCATGA (BspH I), as the 3′-end terminal two consecutive antisense codons for each antisense oligonucleotide at any given length. The length of the entire antisense sequence (n) of each antisense oligonucleotide including selected antisense sequence of orientation (m) in within was measured by antisense codon. n is an integer. m is an integer. n>m. m=2. 5′-TCATGA (BspH I) is selected antisense sequence of orientation within the entire antisense sequence of each antisense oligonucleotide of the library. The length of selected antisense sequence of orientation (m) was measured by antisense codon. As will be apparent to one of skilled in the art, antisense restriction endonuclease recognition sequences include antisense termination codons within their antisense recognition sequence are included in the library. The result of this arrangement is that these antisense oligonucleotides will preferentially hybridize to regions of non-template (sense) strand of genomic DNA, or mRNA or 2^{nd} single strand of cDNA downstream of and including a two-codon-restriction-enzyme-recognition sequence, such as 5′-TCATGA (BspH I) within 5′-UTR or downstream of and including an two-codon restriction endonuclease recognition sequence, such as 5′-TCATGA (BspH I), within 3′-UTR regions in 5′ towards 3′ direction due to the fact that antisense sequences corresponding to termination codons are specifically included. As will be appreciated by ordinary skilled in the art, in accordance with Watson-Crick DNA complementary rule, a corresponding sense-codon-based oligonucleotide library was being constructed as well and vice versa.

### Example 12

3′-End Start Codon Oriented 5′-UTR Sense Oligonucleotide Library Construction

A library with 3′-end start codon orientation was constructed. For example, a library of oligonucleotides consists of all possible combinations of 64 codons with a start codon, such as 5′-ATG, as 3′-end terminal codon for each oligonucleotide at a given length. The length of the entire sequence (n) of each oligonucleotide including selected sequence of orientation (m) in within was measured by codon. n is an integer. m is an integer. n>m. m=1. 5′-ATG is selected sequence of orientation within the entire sequence of each oligonucleotide of the library. The length of selected sequence of orientation (m) was measured by codon. As will be appreciated by one of skilled in the art, the result of this arrangement is that the oligonucleotides will preferentially hybridize to Antisense 5′-Untranslated Region (Antisense 5′-UTR) of template strand (antisense) of genomic DNA, or 1^{st} single strand of cDNA downstream of and including an antisense start codon such as 5′-CAT of antisense ORF and within Antisense 5′-UTR in 5′ towards 3′ direction due to the fact that sequences corresponding to termination codons are specifically included. As will be appreciated by ordinary skilled in the art, in accordance with Watson-Crick DNA complementary rule, a corresponding antisense-codon-based oligonucleotide library was being constructed as well and vice versa.

### Example 13

3′-End Antisense Promoter Sequence Oriented 5′-UTR Antisense Oligonucleotide Library Construction

A library with 3′-end antisense promoter sequence orientation was constructed. For example, a library of antisense oligonucleotides consists of all possible combinations of 64 antisense codons with an antisense promoter sequence, such as 5′-TTTTATA-3′ as the 3′-end terminal antisense codon for each antisense oligonucleotide at a given length. The length of the entire antisense sequence of each antisense oligonucleotide includes selected antisense sequence of orientation. 5′-TTTTATA-3′ is selected antisense sequence of orientation within the entire antisense sequence of each antisense oligonucleotide of the library. The length of selected antisense sequence of orientation (m) was measured by antisense codon. As will be appreciated by one of skilled in the art, these antisense oligonucleotides will preferentially hybridize to 5′-Untranslated Region (5′-UTR) of non-template strand (sense) of genomic DNA, or mRNA or 2^{nd} single strand of cDNA downstream of and including a promoter sequence, such as 5′-TATAAAA-3′ within 5′-UTR in 5′ towards 3′ direction due to the fact that antisense sequences corresponding to antisense promoter sequence are specifically included. As will be appreciated by ordinary skilled in the art, in accordance with Watson-Crick DNA complementary rule, a corresponding sense-codon-based oligonucleotide library was being constructed as well and vice versa.

### Example 14

3′-End Antisense Enhancer Sequence Oriented Coding Region Antisense Oligonucleotide Library Construction

A library with 3′-end antisense enhancer sequence orientation was constructed. For example, a library of antisense oligonucleotides consists of all possible combinations of 61 antisense codons with an antisense enhancer sequence, such as 5′-CCGCCC-3′ as the 3′-end terminal antisense codon for each antisense oligonucleotide at a given length. The length of the entire antisense sequence of each antisense oligonucleotide includes selected antisense sequence of orientation. 5′-CCGCCC-3′ is selected antisense sequence of orientation (m) within the entire antisense sequence of each antisense oligonucleotide of the library. The length of the entire antisense sequence (n) of each antisense oligonucleotide including selected

antisense-sequence of orientation (m) in within was measured by antisense-codon. n is an integer. m is an integer. n>m. m=2. The length of selected antisense-sequence of orientation (m) was measured by antisense-codon. As will be appreciated by one of skilled in the art, these antisense oligonucleotides will preferentially hybridize to coding region of non-template strand (sense) of genomic DNA, or pre-mRNA downstream of and including an enhancer sequence, such as 5′-GGGCGG-3′ within a coding region in 5′ towards 3′ direction due to the fact that antisense sequences corresponding to antisense enhancer sequence are specifically included. As will be appreciated by ordinary skilled in the art, in accordance with Watson-Crick DNA complementary rule, a corresponding sense-codon-based oligonucleotide library was being constructed as well and vice versa.

### Example 15

5′-End Antisense Start Codon Oriented 5′-UTR Antisense Oligonucleotide Library Construction

A library with 5′-end antisense start codon orientation was constructed. For example, a library of antisense oligonucleotides consists of all possible combinations of 64 antisense codons with an antisense start codon, such as 5′-CAT, as the 5′-end terminal antisense codon for each antisense oligonucleotide at a given length. The length of the entire antisense sequence (n) of each antisense oligonucleotide including selected antisense sequence of orientation (m) in within was measured by antisense codon. n is an integer. m is an integer. n>m. m=1. 5′-CAT is selected antisense sequence of orientation within the entire antisense sequence of each antisense oligonucleotide of the library. The length of selected antisense sequence of orientation (m) was measured by antisense codon. As will be appreciated by one of skilled in the art, these antisense oligonucleotides will preferentially hybridize to 5′-Untranslated Region (5′-UTR) of non-template strand (sense) of genomic DNA, or mRNA or 2^{nd} single strand of cDNA upstream of and including a start codon such as 5′-ATG of ORF and within 5′-UTR in 5′ towards 3′ direction due to the fact that antisense sequences corresponding to antisense termination codons are specifically included. As will be appreciated by ordinary skilled in the art, in accordance with Watson-Crick DNA complementary rule, a corresponding sense-codon-based oligonucleotide library was being constructed as well and vice versa.

### Example 16

5′-End Stop Codon Oriented 3′-UTR Sense Oligonucleotide Library Construction

A library with 5′-end stop codon orientation was constructed. For example, a library of oligonucleotides consists of all possible combinations of 64 codons with a stop codon, such as 5′-TGA, as 5′-end terminal codon for each oligonucleotide at a given length. The length of the entire sequence (n) of each oligonucleotide including selected sequence of orientation (m) in within was measured by codon. n is an integer. m is an integer. n>m. m=1. 5′-TGA is selected sequence of orientation within the entire sequence of each oligonucleotide of the library. The length of selected sequence of orientation (m) was measured by codon. As will be appreciated by one of skilled in the art, the result of this arrangement is that the oligonucleotides will preferentially hybridize to Antisense 3′-Untranslated Region (Antisense 3′-UTR) of template strand (antisense) of genomic DNA, or 1^{st} single strand of cDNA upstream of and including an antisense stop codon, such as 5′-TCA of antisense ORF and within the antisense 3′-UTR in 5′ towards 3′ direction due to the fact that sequences corresponding to termination codons are specifically included. As will be appreciated by ordinary skilled in the art, in accordance with Watson-Crick DNA complementary rule, a corresponding antisense-codon-based oligonucleotide library was being constructed as well and vice versa.

### Example 17

3′-End Antisense Stop Codon Oriented 3′-UTR Antisense Oligonucleotide Library Construction

A library with 3′-end antisense stop codon orientation was constructed. For example, a library of antisense oligonucleotides consists of all possible combinations of 64 antisense codons with an antisense stop codon, such as 5′-TCA, as the 3′-end terminal antisense codon for each antisense oligonucleotide at a given length. The length of the entire antisense sequence (n) of each antisense oligonucleotide including selected antisense sequence of orientation (m) in within was measured by antisense codon. n is an integer. m is an integer. n>m. m=1. 5′-TCA is selected antisense sequence of orientation within the entire antisense sequence of each antisense oligonucleotide of the library. The length of selected antisense sequence of orientation (m) was measured by antisense codon. As will be appreciated by one of skilled in the art, these antisense oligonucleotides will preferentially hybridize to 3′-Untranslated Region (3′-UTR) of non-template strand (sense) of genomic DNA, or mRNA or 2^{nd} single strand of cDNA downstream of and including a stop codon such as 5′-TGA of ORF and within 3′-UTR in 5′ towards 3′ direction due to the fact that antisense sequences corresponding to antisense termination codons are specifically included. As will be appreciated by ordinary skilled in the art, in accordance with Watson-Crick DNA complementary rule, a corresponding sense-codon-based oligonucleotide library was being constructed as well and vice versa.

### Example 18

5′-End Oligo-d(T)ₛ Oriented 3′-UTR Antisense Oligonucleotide Library Construction

A library with 5′-end oligo(T)ₛ orientation was constructed. For example, a library of antisense oligonucleotides consists of all possible combinations of 64 antisense codons with an oligo(T)ₛ, such as six-antisense-codon-long oligo-d(T)ₛ, as the 5′-end terminal antisense codons for each antisense oligonucleotide at a given length. The length of the entire antisense sequence (n) of each antisense oligonucleotide including selected antisense sequence of orientation (m) in within was measured by antisense codon. n is an integer. m is an integer. n>m. m=1. 5′-oligo-d(T)ₛ is selected antisense sequence of orientation within the entire antisense sequence of each antisense oligonucleotide of the library. The length of selected rmined antisense sequence of orientation (m) was measured by antisense codon. As will be appreciated by one of skilled in the art, these antisense oligonucleotides will preferentially hybridize to 3′-Untranslated Region (3′-UTR) of non-template strand (sense) of genomic DNA, or mRNA or 2^{nd} single strand of cDNA upstream of and including a poly(A) such as 3′-six-antisense-codon-long poly(A) and within 3′-UTR in 5′ towards 3′ direction due to the fact that antisense sequences corresponding to antisense termination codons are specifically included. As will be appreciated by ordinary skilled in the art, in accordance with Watson-Crick DNA complementary rule, a corresponding sense-codon-based oligonucleotide library was being constructed as well and vice versa.

### Example 19

3′-End Poly(A) Oriented 3′-UTR Sense Oligonucleotide Library Construction

A library with 3′-end poly(A) orientation was constructed. For example, a library of sense oligonucleotides consists of all possible combinations of 64 sense codons with a poly(A), such as six-antisense-codon-long poly(A), as the 3′-end terminal codons for each sense oligonucleotide at a given length. The length of the entire sequence (n) of each oligonucleotide including selected sequence of orientation (m) in within was measured by codon. n is an integer. m is an integer. n>m. m=1. 3′-poly(A)_{S} is selected sequence of orientation within the entire sequence of each sense oligonucleotide of the library. The length of selected sequence of orientation (m) was measured by codon. As will be appreciated by one of skilled in the art, these sense oligonucleotides will preferentially hybridize to Antisense 3′-Untranslated Region (Antisense 3′-UTR) of template strand (antisense) of genomic DNA, or 1^{st} single strand of cDNA upstream of and including an Oligo-d(T)_{S}, such as 3′-six-codon-long Oligo-d(T)_{S} and within 3′-UTR in 5′ towards 3′ direction due to the fact that sequences corresponding to termination codons are specifically included. As will be appreciated by ordinary skilled in the art, in accordance with Watson-Crick DNA complementary rule, a corresponding antisense-codon-based oligonucleotide library was being constructed as well and vice versa.

### Example 20

3′-End 5′-TAC Oriented Pre-mRNA 5′-Splice Donor Site Antisense Oligonucleotide Library Construction

A library with 3′-end terminal antisense codon selected from a group of antisense codons comprising 5′-TAC, 5′-GAC, 5′-CAC and 5′-AAC as antisense sequence of orientation was constructed. For example, a library of antisense oligonucleotides consists of all possible combinations of 64 antisense codons with an antisense codon selected from a group of antisense codons comprising 5′-TAC, 5′-GAC, 5′-CAC and 5′-AAC, such as 5′-TAC as 3′-end terminal antisense codons for each antisense oligonucleotide at a given length. The length of the entire antisense sequence (n) of each antisense oligonucleotide including selected antisense sequence of orientation (m) in within was measured by antisense codon. n is an integer. m is an integer. n>m. m=1. 5′-TAC is selected antisense sequence of orientation within the entire antisense sequence of each antisense oligonucleotide of the library. The length of selected antisense sequence of orientation (m) was measured by antisense codon. As will be appreciated by one of skilled in the art, these antisense oligonucleotides will preferentially hybridize to an intron of Pre-mRNA or non-template strand (sense) of genomic DNA downstream of and including 5′-GUA and within of an intron of Pre-mRNA in 5′ towards 3′ direction due to the fact that antisense sequences corresponding to antisense termination codons are specifically included. As will be appreciated by ordinary skilled in the art, in accordance with Watson-Crick DNA complementary rule, a corresponding sense-codon-based oligonucleotide library was being constructed as well and vice versa.

### Example 21

5′-End 5′-CTT Oriented Pre-mRNA 3′-Splice Acceptor Site Antisense Oligonucleotide Library Construction

A library with 5′-end terminal antisense codon selected from a group of antisense codons comprising 5′-CTT/5′-CUU, 5′-CTG/5′-CUG, 5′-CTC/5′-CUC and 5′-CTA/5′-CUA as antisense sequence of orientation was constructed. For example, a library of antisense oligonucleotides consists of all possible combinations of 64 antisense codons with an antisense codon selected from a group of antisense codons comprising 5′-CTT/5′-CUU, 5′-CTG/5′-CUG, 5′-CTC/5′-CUC and 5′-CTA/5′-CUA, such as 5′-CTT as 5′-end terminal antisense codons for each antisense oligonucleotide at a given length. The length of the entire antisense sequence (n) of each antisense oligonucleotide including selected antisense sequence of orientation (m) in within was measured by antisense codon. n is an integer. m is an integer. n>m. m=1. 5′-CTT is selected antisense sequence of orientation within the entire antisense sequence of each antisense oligonucleotide of the library. The length of selected antisense sequence of orientation (m) was measured by antisense codon. As will be appreciated by one of skilled in the art, these antisense oligonucleotides will preferentially hybridize to an intron of Pre-mRNA or non-template strand (sense) of genomic DNA upstream of and including 5′-AAG and within of an intron of Pre-mRNA in 5′ towards 3′ direction due to the fact that antisense sequences corresponding to antisense termination codons are specifically included. As will be appreciated by ordinary skilled in the art, in accordance with Watson-Crick DNA complementary rule, a corresponding sense-codon-based oligonucleotide library was being constructed as well and vice versa.

### Example 22

PCR Protocol

1 to 25 ng cDNA, 1.5 mM MgCl₂, 50 mM KCl, 20 mM Tris-HCl (pH 7.4), 0.1 mM EDTA, 0.1 mM DTT, 150 uM dNTPs (dATP, dCTP, dGTP and dTTP), 0.05% Tween 20, 10 to 25 pM primer and 1 to 2 units of Taq DNA polymerase in 20 ul. Thermostable DNA polymerase was selected from a group of polymerases which includes, without limiting the generality of the foregoing, Taq DNA polymerase, AmpliTaq Gold DNA polymerase, Pfu DNA polymerase, Tfl DNA polymerase, Tli DNA polymerase, Tth DNA polymerase, Vent_{R} (exo⁻) DNA polymerase and Deep Vent_{R} (exo⁻) DNA polymerase. The analogues and modified dNTPs may be used in conjunction with the present invention which include, without limiting the generality of the foregoing, 5′-nitroindole, 3′-nitropyrrole, inosine, hypoxanthine, LNA, Peptide Nucleic Acid (PNA), Morpholino phosphoroamidate (MF), 2′-O-Methoxyethyl oligonucleotide(s) (2′-MOE), 2′-0-Methyl (2′-OME), Phosphorothioate (PS), Phosphoroamidate, Methylphosphonate, biotin-11-dUTP, biotin-16-dUTP, 5′-bromo-dUTP, dUTP, dig-11-dUTP and 7-deaza dGTP.

### Example 23

PCR Temperature Profiles

The threshold cycle consists of denaturing temperature of 45 second at 94° C., annealing temperature of 90 second at 40° C. and extension temperature of 60 second at 72° C. The number of cycles for PCR amplification was 30, each of which consists of a denaturing step of 30 seconds at 94° C., an annealing step of 90 seconds at 40° C. and an extension step of 60 seconds at 72° C. The end cycle consists of 5 minutes at 72° C. following by 4° C. Each specified upstream primer is a distinct 9 mers 5′-ATG oriented oligonucleotide represented by the formula 5′-I_{S}(C_{S})ₙ₁-3′. The common downstream primer is oligo-d(T)₁₈.

### (1) Denaturation:

94° C. for 30 sec.: It is applicable to all the said primers

### (2) Annealing:

40° C. or 40° C. plus 1-5° C. or 40° C. minus 1-5° C. for 60 sec.

It is applicable to the said 49 upstream primers having 11.1% GC content after the incorporation of seven LNA in each 9-mer oligonucleotide sequence, such as 5′-ATGATAATA. It is applicable to the said 308 upstream primers having 22.2% GC content after the incorporation of six LNA in each 9-mer oligonucleotide sequence, such as 5′-ATGGAAATA. It is applicable to the said 820 upstream primers having 33.3% GC content after the incorporation of four LNA in each 9-mer oligonucleotide sequence, such as 5′-ATGGCAATA. It is applicable to the said 1,168 upstream primers having 44.4% GC content after the incorporation of two LNA in each 9-mer oligonucleotide sequence, such as 5′-ATGGCAGAA. It is applicable to the said 928 upstream primers having 55.6% GC content after the incorporation of one LNA in each 9-mer oligonucleotide sequence, such as 5′-ATGGCAGCA. It is applicable to the said 384 upstream primers having 66.7% GC content after without the incorporation of LNA in each 9-mer oligonucleotide sequence, such as 5′-ATGGCAGCC. It is applicable to the said 64 upstream primers having 77.8% GC content after the incorporation of one LNA at 5′-end of each 9-mer oligonucleotide sequence, such as 5′-ATGGCCGCC.

### (3) Extension: 72° C. for 60 sec.

### (4) Cycle Number: 30

(5) Final Extension: 72° C. for 5 minus for all

If no bands on an Agarose gel are observed, the annealing temperature might be adjusted in the range of 1° C. to 5° C. below the original annealing temperature and, if unwanted bands and/or several bands appeared, the annealing temperature might be adjusted in the range of 1° C. to 5° C. above the original annealing temperature in each subsequent optimization step. It is recommended that if the inventive 9 mers, 12 mers, 15 mers, 18 mers, 21 mers, and 24 mers oligonucleotides are used as the PCR primers, the range of annealing temperatures is often from 37° C. to 56° C. The higher the annealing temperature is increased, the more specific the PCR results may obtain. Therefore, the annealing temperature can be increased as high as the extension temperature in some cases under certain conditions.

### Example 24

The Touchdown PCR Protocol:

The Touchdown PCR protocol starts with an annealing temperature above the primer's ideal temperature. At each cycle, the annealing temperature is programmed to decrease 1° C. until reaching the targeting annealing temperature. In one preferred embodiment, 9 mer 5′-ATG oriented oligonucleotides represented by the formula 5′-I_{S}(C_{S})ₙ₁-3′ such as 5′-ATGGCCGCC had three consecutive universal bases such as 5′-nitroindoles covalently added at each of their 5′-ends to form 12 mer oligonucleotides. The 12 mer oligonucleotides were then used as PCR upstream primer. oligo-d(T)₁₈ was used as PCR downstream primer. In one preferred embodiment, the threshold cycle consists of a denaturing step of 45 seconds at 94° C. The second cycle consists of denaturing step of 30 seconds at 94° C., an annealing step of 90 seconds at 50° C. and an extension step of 60 seconds at 72° C. The third cycle consists of a denaturing step of 30 seconds at 94° C., an annealing step of 90 seconds at 49° C. and an extension step of 60 seconds at 72° C. The fourth cycle consists of a denaturing step of 30 seconds at 94° C., an annealing step of 90 seconds at 48° C. and an extension step of 60 seconds at 72° C. The fifth cycle consists of a denaturing step of 30 seconds at 94° C., an annealing step of 90 seconds at 47° C. and an extension step of 60 seconds at 72° C. The sixth cycle consists of a denaturing step of 30 seconds at 94° C., an annealing step of 90 seconds at 46° C. and an extension step of 60 seconds at 72° C. The seventh cycle consists of a denaturing step of 30 seconds at 94° C., an annealing step of 90 seconds at 45° C. and an extension step of 60 seconds at 72° C. The eighth cycle consists of a denaturing step of 30 seconds at 94° C., an annealing step of 90 seconds at 44° C. and an extension step of 60 seconds at 72° C. The ninth cycle consists of a denaturing step of 30 seconds at 94° C., an annealing step of 90 seconds at 43° C. and an extension step of 60 seconds at 72° C. The tenth cycle consists of a denaturing step of 30 seconds at 94° C., an annealing step of 90 seconds at 42° C. and an extension step of 60 seconds at 72° C. The number of cycles for subsequent PCR amplification was 30, with each cycle consisting of a denaturing step of 30 seconds at 94° C., an annealing step of 90 seconds at 42° C. and an extension step of 60 seconds at 72° C. The final cycle consists of 5 minutes at 72° C. following by 4° C.

### Example 25

Mitochondrial DNA Isolation

Prepare extraction buffer (0.4M mannitol, 1 mM ethylene glycol-bis[aminoethyl ether] N′,N′,N′,N′, -tetraacetic acid (EGTA), 15 mM N-[2-hydroxyethyl]piperazine-N′-[ethanesulfonic acid] (HEPES), 15 mM diethyldithiocarbamic acid (DIECA), 0.1% bovine serum albumin, 0.05% cysteine, 0.5% Polyclar AT, pH 7.4). Glassware and Buffers were autoclaved prior to use. Grind cell culture in mortar and pestle or Waring blender with extraction buffer at 4° C. After filtration through two layers of Miracloth, the homogenate is centrifuged at 150 g for 10-15 minutes. The supernatant is then centrifuged three times at 3,000 g for 10-15 minutes to separate cellular debris, nuclei and proplastids from the mitochondria. Mitochondria are pelleted at 10,000 g for 20-30 minutes at 4° C., resuspended in DNase buffer (0.4M mannitol, 10 mM magnesium chloride, 15 mM HEPES, pH 7.4) and treated with DNase I (0.1 mg/mL) at 4° C. for 60 minutes. Washing the mitochondria with DNase inhibiting buffer (0.4M mannitol, 15 mM HEPES, 100 mM EGTA, pH 7.4), the isolated mitochondria are further purified by centrifugation in a discontinuous Percoll gradient (45%, 21%, 14% Percoll) at 15,000 g for 15-20 minutes. The mitochondria that band are pooled and diluted with resuspension buffer (0.4M mannitol, 15 mM HEPES, 10 mM EGTA, 15 mM DIECA, pH 7.4,) and centrifuged three times at 10,000 g for 10-15 minutes at 4° C. to remove Percoll. The washed and pelleted mitochondria are resuspended in lysis buffer (0.1M Tris. HCl, 0.1M NaCl, 0.05M EDTA, 1% sarcosyl, 1% sodium dodecyl sulfate, pH 8.0) and incubated at 65° C. for 30 minutes. Organic material is removed by addition of 5M potassium acetate with incubation on ice for 20 minutes. Following the centrifugation, the supernatant is mixed with an equal volume of isopropanol. Precipitated mitochondria DNA is dissolved in 10 mM TE buffer. The precipitated mitochondria DNA is further purified by extraction with phenol (buffered with TE), followed by three extractions with chloroform:isoamyl alcohol (24:1 v/v), reprecipitated and dissolved in TE buffer and stored at −70° C. RNA is removed by addition of RNase during the restriction endonuclease digestion of the mitochondrial DNA.

While the preferred embodiments and examples of the invention have been described above, it will be recognized and understood that various modifications may be made therein, and the appended claims are intended to cover all such modifications which may fall within the spirit and scope of the invention.

### XIV. EQUIVALENTS

While the preferred embodiments of the invention have been described above, it shall be recognized and understood that various modifications may be made therein, and the appended claims are intended to cover all such modifications that may fall within the spirit and scope of the invention. Taken together, the inventive methods, without limiting the generality of the foregoing, comprise a series of complex and combinatorial methods, working platforms and systems. A genome-wide antisense oligonucleotides have been described through the foregoing detailed illustrations and descriptions of various aspects, different examples and specific embodiments of the present invention. Although the specific embodiments and examples have been introduced and disclosed herein, it has been accomplished by way of example for the objectives of explanation and illustration only, without limiting the generality of the foregoing, regarding the spirit and scope of the claims made for the invention. Specifically, it is contemplated by the inventors that various substitutions, alterations, modification, revisions and developments may be made in part or as the whole regarding both the structures or and the functions of the invention without departing from the spirit and the scope of the invention as defined by the claims. For example, the choices of nucleotides and amino acids from natural, synthetic or chemically modified resources respectively, the form of nucleic acids strands, such as sense strand and antisense strand, the forms of genomic DNA, cDNA, RNA, pre-mRNA, mRNA, RNA-DNA hybrid, oligonucleotide, deoxyoligonucleotide, peptide, their corresponding analogues and derivatives, the forms of being attached or associate or linked or immobilized at a specific discrete position on or to a suitable carrier whether covalently or non-covalently, the forms of being attached or associate or linked or immobilized at a specific discrete position on or to a suitable carrier whether directly or indirectly, the forms of being attached or associate or linked or immobilized at a specific discrete position at a specific discrete position on or to a suitable carrier whether through or not through a linker, the size and shape of the said specific discrete position, the size and shape of the said suitable carrier, the forms and shape of the said linker, the particular labeling substances and the corresponding signal detection measurements or the particular single, individual or combinatorial oligonucleotides or deoxyoligonucleotides or RNAs or DNAs or RNA-DNA hybrids or peptides are conceived as a matter of routine for one skilled in the art with knowledge of the embodiments described herein.

TABLE 1
Comparison of Antisense Codon-based Oligonucleotide and Nucleotide-based Oligonucleotide Libraries

| | | | | Ratio |
|---|---|---|---|---|
| | | | | Nucleotide/ |
| Antisense Codon-based Oligonucleotide Library* | | Nucleotide-based Oligonucleotide Library** | | Antisense |
| Length | Total Number | Length | Total Number | Codon |
| 1 Antisense Codon | 61⁽¹⁻¹⁾ = 1 | 3mer | 4^{3×1} = 64 | 64.00 |
| 2 Antisense Codons | 61⁽²⁻¹⁾ = 61 | 6mer | 4^{3×2} = 4,096 | 67.15 |
| 3 Antisense Codons | 61⁽³⁻¹⁾ = 3,721 | 9mer | 4^{3×3} = 262,144 | 70.45 |
| 4 Antisense Codons | 61⁽⁴⁻¹⁾ = 226,981 | 12mer | 4^{3×4} = 16,777,216 | 73.91 |
| 5 Antisense Codons | 61⁽⁵⁻¹⁾ = 13,845,841 | 15mer | 4^{3×5} = 1,073,741,824 | 77.55 |
| 6 Antisense Codons | 61⁽⁶⁻¹⁾ = 844,596,301 | 18mer | 4^{3×6} = 68,719,476,736 | 81.36 |
| 7 Antisense Codons | 61⁽⁷⁻¹⁾ = 51,520,374,361 | 21mer | 4^{3×7} = 4,398,046,511,104 | 85.37 |
| 8 Antisense Codons | 61⁽⁸⁻¹⁾ = 3,142,742,836,021 | 24mer | 4^{3×8} = 281,474,976,710,656 | 89.56 |
| 9 Antisense Codons | 61⁽⁹⁻¹⁾ = 191,707,312,997,281 | 27mer | 4^{3×9} = 18,014,398,509,481,984 | 93.97 |
| 10 Antisense Codons | 61⁽¹⁰⁻¹⁾ = 11,694,146,092,834,141 | 30mer | 4^{3×10} = 1,152,921,504,606,846,976 | 98.59 |
| 11 Antisense Codons | 61⁽¹¹⁻¹⁾ = 713,342,911,662,882,601 | 33mer | 4^{3×11} = 73,786,976,294,838,206,464 | 103.44 |
| 12 Antisense Codons | 61⁽¹²⁻¹⁾ = 43,513,917,611,435,838,661 | 36mer | 4^{3×12} = 4,722,366,482,869,645,213,696 | 108.53 |
| 13 Antisense Codons | 61⁽¹³⁻¹⁾ = 2,654,348,974,297,586,158,321 | 39mer | 4^{3×13} = 302,231,454,903,657,293,676,544 | 113.86 |
| 14 Antisense Codons | 61⁽¹⁴⁻¹⁾ = 161,915,287,432,152,755,657,581 | 42mer | 4^{3×14} = 19,342,813,113,834,066,795,298,816 | 119.46 |
| 15 Antisense Codons | 61⁽¹⁵⁻¹⁾ = 9,876,832,533,361,318,095,112,441 | 45mer | 4^{3×15} = 1,237,940,039,285,380,274,899,124,224 | 125.34 |
| 16 Antisense Codons | 61⁽¹⁶⁻¹⁾ = 602,486,784,535,040,403,801,858,901 | 48mer | 4^{3×16} = 79,228,162,514,264,337,593,543,950,336 | 131.50 |
| n Antisense Codons | 61^{(n-m)} = 61⁽ⁿ⁻¹⁾ = (4³ − 3)⁽ⁿ⁻¹⁾ | 3n mer | 4³ⁿ 4³ⁿ/61⁽ⁿ⁻¹⁾ or 4³ⁿ/(4³ − 3)⁽ⁿ⁻¹⁾ | |
| Formulas: | 61^{(n-m)} = 61⁽ⁿ⁻¹⁾ = (4³ − 3)⁽ⁿ⁻¹⁾ | 3n mer | 4³ⁿ 4³ⁿ/61⁽ⁿ⁻¹⁾ | |

*All Possible Combinations of 61 antisense codons, 61^{(n-m)} = 61⁽ⁿ⁻¹⁾, n > m, m = 1.
**All Possible Combinations of Four Nucleotides (A.T.G.C) or Four Bases

TABLE 2
Classification of Antisense Oligonucleotide by GC Content

| | Antisense Codon No. | | | | | | |
|---|---|---|---|---|---|---|---|
| | 2 | 3 | 4 | 5 | 6 | 7 | 8 |
| Item | Length | | | | | | |
| GC Content | 6 mer | 9 mer | 12 mer | 15 mer | 18 mer | 21 mer | 24 mer |
| 0 | 0% | 0% | 0% | 0% | 0% | 0% | 0% |
| 1 | 16.67% | 11.11% | 8.33% | 6.67% | 5.56% | 4.76% | 4.12% |
| 2 | 33.33% | 22.22% | 16.67% | 13.33% | 11.11% | 9.52% | 8.33% |
| 3 | 50.00% | 33.33% | 25.00% | 20.00% | 16.67% | 14.29% | 12.50% |
| 4 | 66.67% | 44.44% | 33.33% | 26.67% | 22.22% | 19.05% | 16.67% |
| 5 | 83.33% | 55.56% | 41.67% | 33.33% | 27.78% | 23.81% | 20.83% |
| 6 | 100% | 66.67% | 50.00% | 40.00% | 33.33% | 28.57% | 25.00% |
| 7 | | 77.78% | 58.33% | 46.67% | 38.89% | 33.33% | 29.17% |
| 8 | | 88.89% | 66.67% | 53.33% | 44.44% | 38.10% | 33.33% |
| 9 | | 100% | 75.00% | 60.00% | 50.00% | 42.86% | 37.50% |
| 10 | | | 83.33% | 66.67% | 55.56% | 47.62% | 41.67% |
| 11 | | | 91.67% | 73.33% | 61.11% | 52.38% | 45.83% |
| 12 | | | 100% | 80.00% | 66.67% | 57.14% | 50.00% |
| 13 | | | | 86.67% | 72.22% | 61.90% | 54.17% |
| 14 | | | | 93.33% | 77.78% | 66.67% | 58.33% |
| 15 | | | | 100% | 83.33% | 71.43% | 62.50% |
| 16 | | | | | 88.89% | 76.19% | 66.67% |
| 17 | | | | | 94.44% | 80.95% | 70.83% |
| 18 | | | | | 100% | 85.71% | 75.00% |
| 19 | | | | | | 90.48% | 79.17% |
| 20 | | | | | | 95.24% | 83.33% |
| 21 | | | | | | 100% | 87.50% |
| 22 | | | | | | | 91.67% |
| 23 | | | | | | | 95.83% |
| 24 | | | | | | | 100.00% |

What is claimed is:

1: A method of generating an antisense oligonucleotide library comprising a plurality of antisense oligonucleotides, wherein said antisense oligonucleotide library has a complexity according to an algorithm, wherein said algorithm is 61^{(n-m)}, wherein 61 represents the number of antisense amino acid coding codons, wherein the length of said antisense oligonucleotides has (n−m) antisense—codon-length long, wherein said n represents the length of said antisense oligonucleotides measured by antisense codon, wherein said antisense oligonucleotides have antisense sequence of orientation, wherein the said antisense sequence of orientation consists of a selected antisense sequence, wherein the length of said antisense sequence of orientation has m-antisense-codon-length long, wherein said m represents the length of said antisense sequence of orientation measured by antisense codon, wherein n is an integer, wherein n>zero, wherein m is an integer, wherein m>zero, wherein n>m, wherein (n−m) represents n minus m, wherein n−m<9, wherein (n−m) represents the entire length of said antisense oligonucleotide, wherein 61^{(n-m)} represents the number of antisense oligonucleotide in said library.

2: A method of generating an antisense mitochondria oligonucleotide library comprising a plurality of antisense oligonucleotides, wherein said antisense oligonucleotide library has a complexity according to an algorithm, wherein said algorithm is 60^{(n-m)}, wherein 60 represents the number of antisense mitochondria codons, wherein the length of said antisense oligonucleotides has (n-m) antisense—codon-length long, wherein said n represents the length of said antisense oligonucleotides measured by antisense codon, wherein said antisense oligonucleotides have antisense sequence of orientation, wherein the said antisense sequence of orientation consists of a selected antisense sequence, wherein the length of said antisense sequence of orientation has m-antisense-codon-length long, wherein said m represents the length of said antisense sequence of orientation measured by antisense codon, wherein n is an integer, wherein n>zero, wherein m is an integer, wherein m>zero, wherein n>m, wherein (n−m) represents n minus m, wherein n−m<9, wherein (n−m) represents the entire length of said antisense oligonucleotide, wherein 60^{(n-m)} represents the number of antisense mammalian mitochondria oligonucleotide in said library.

3: An antisense oligonucleotide library was generated according to claim 1, wherein each said antisense oligonucleotide further comprises a linker at either 5′-end or 3′-end of said antisense oligonucleotides; wherein said linker being selected from a group consisting antisense sense initiation codons; antisense termination codon; antisense amino acid coding codon; two consecutive antisense codons consisting an antisense restriction enzyme site; and combinations thereof.

4: An antisense oligonucleotide library was generated according to claim 1, wherein n−m=2, wherein said oligonucleotides are grouped according to GC content, wherein said GC content are selected from a group consisting of 16.67% GC content, 33.33% GC content, 50.00% GC content, 66.67% GC content, 83.33% GC content and 100.00% GC content.

5: An antisense oligonucleotide library was generated according to claim 1, wherein n−m=3, wherein said oligonucleotides are grouped according to GC content, wherein said GC content are selected from a group consisting of 11.11% GC content, 22.22% GC content, 33.33% GC content, 44.44% GC content, 55.56% GC content, 66.67% GC content, 77.78% GC content, 88.89 GC content and 100.00% GC content.

6: An antisense oligonucleotide library was generated according to claim 1, wherein n−m=4, wherein said oligonucleotides are grouped according to GC content, wherein said GC content are selected from a group consisting of 8.33% GC content, 16.67% GC content, 25.00% GC content, 33.33% GC content, 41.67% GC content, 50.00% GC content, 58.33% GC content, 66.67% GC content, 75.00% GC content, 83.33 GC content, 91.67% GC content and 100.00% GC content.

7: An antisense oligonucleotide library was generated according to claim 1, wherein n−m=5, wherein said oligonucleotides are grouped according to GC content, wherein said GC content are selected from a group consisting of 6.67% GC content, 13.33% GC content, 20.00% GC content, 26.67% GC content, 33.33% GC content, 40.00% GC content, 46.67% GC content, 53.33% GC content, 60.00% GC content, 66.67% GC content, 73.33% GC content, 80.00% GC content, 86.67 GC content, 93.33% GC content and 100.00% GC content.

8: An antisense oligonucleotide library was generated according to claim 1, wherein n−m=6, wherein said oligonucleotides are grouped according to GC content, wherein said GC content are selected from a group consisting of 5.56% GC content, 11.11% GC content, 16.67% GC content, 22.22% GC content, 27.78% GC content, 33.33% GC content, 38.89% GC content, 44.44% GC content, 50.00% GC content, 55.56% GC content, 61.11% GC content, 66.67% GC content, 72.22% GC content, 77.78% GC content, 83.33% GC content, 88.89 GC content, 94.44% GC content and 100.00% GC content.

9: An antisense oligonucleotide library was generated according to claim 1, wherein n−m=7, wherein said oligonucleotides are grouped according to GC content, wherein said GC content are selected from a group consisting of 4.76% GC content, 9.52% GC content, 14.29% GC content, 19.05% GC content, 23.81% GC content, 28.57% GC content, 33.33% GC content, 38.10% GC content, 42.86% GC content, 47.62% GC content, 52.38% GC content, 57.14% GC content, 61.90% GC content, 66.67% GC content, 71.43% GC content, 76.19% GC content, 80.95% GC content, 85.71 GC content, 90.48% GC content, 95.24% GC content and 100.00% GC content.

10: An antisense oligonucleotide library was generated according to claim 1, wherein n−m=8, wherein said oligonucleotides are grouped according to GC content, wherein said GC content are selected from a group consisting of 4.12% GC content, 8.33% GC content, 12.50% GC content, 16.67% GC content, 20.83% GC content, 25.00% GC content, 29.17% GC content, 33.33% GC content, 37.50% GC content, 41.67% GC content, 45.83% GC content, 50.00% GC content, 54.17% GC content, 58.33% GC content, 62.50% GC content, 66.67% GC content, 70.83% GC content, 75.00% GC content, 79.17% GC content, 83.33% GC content, 87.50% GC content, 91.67% GC content, 95.83% GC content and 100% GC content.

11: An antisense oligonucleotide library was generated according to claim 3, wherein n−m=2, wherein said oligonucleotides are grouped according to GC content, wherein said GC content are selected from a group consisting of 16.67% GC content, 33.33% GC content, 50.00% GC content, 66.67% GC content, 83.33% GC content and 100.00% GC content.

12: An antisense oligonucleotide library was generated according to claim 3, wherein n−m=3, wherein said oligonucleotides are grouped according to GC content, wherein said GC content are selected from a group consisting of 11.11% GC content, 22.22% GC content, 33.33% GC content, 44.44% GC content, 55.56% GC content, 66.67% GC content, 77.78% GC content, 88.89 GC content and 100.00% GC content.

13: An antisense oligonucleotide library was generated according to claim 3, wherein n−m=4, wherein said oligonucleotides are grouped according to GC content, wherein said GC content are selected from a group consisting of 8.33% GC content, 16.67% GC content, 25.00% GC content, 33.33% GC content, 41.67% GC content, 50.00% GC content, 58.33% GC content, 66.67% GC content, 75.00% GC content, 83.33 GC content, 91.67% GC content and 100.00% GC content.

14: An antisense oligonucleotide library was generated according to claim 3, wherein n−m=5, wherein said oligonucleotides are grouped according to GC content, wherein said GC content are selected from a group consisting of 6.67% GC content, 13.33% GC content, 20.00% GC content, 26.67% GC content, 33.33% GC content, 40.00% GC content, 46.67% GC content, 53.33% GC content, 60.00% GC content, 66.67% GC content, 73.33% GC content, 80.00% GC content, 86.67 GC content, 93.33% GC content and 100.00% GC content.

15: An antisense oligonucleotide library was generated according to claim 3, wherein n−m=6, wherein said oligonucleotides are grouped according to GC content, wherein said GC content are selected from a group consisting of 5.56% GC content, 11.11% GC content, 16.67% GC content, 22.22% GC content, 27.78% GC content, 33.33% GC content, 38.89% GC content, 44.44% GC content, 50.00% GC content, 55.56% GC content, 61.11% GC content, 66.67% GC content, 72.22% GC content, 77.78% GC content, 83.33% GC content, 88.89 GC content, 94.44% GC content and 100.00% GC content.

16: An antisense oligonucleotide library was generated according to claim 3, wherein n−m=7, wherein said oligonucleotides are grouped according to GC content, wherein said GC content are selected from a group consisting of 4.76% GC content, 9.52% GC content, 14.29% GC content, 19.05% GC content, 23.81% GC content, 28.57% GC content, 33.33% GC content, 38.10% GC content, 42.86% GC content, 47.62% GC content, 52.38% GC content, 57.14% GC content, 61.90% GC content, 66.67% GC content, 71.43% GC content, 76.19% GC content, 80.95% GC content, 85.71 GC content, 90.48% GC content, 95.24% GC content and 100.00% GC content.

17: An antisense oligonucleotide library was generated according to claim 3, wherein n−m=8, wherein said oligonucleotides are grouped according to GC content, wherein said GC content are selected from a group consisting of 4.12% GC content, 8.33% GC content, 12.50% GC content, 16.67% GC content, 20.83% GC content, 25.00% GC content, 29.17% GC content, 33.33% GC content, 37.50% GC content, 41.67% GC content, 45.83% GC content, 50.00% GC content, 54.17% GC content, 58.33% GC content, 62.50% GC content, 66.67% GC content, 70.83% GC content, 75.00% GC content, 79.17% GC content, 83.33% GC content, 87.50% GC content, 91.67% GC content, 95.83% GC content and 100% GC content.

18: An antisense oligonucleotide library was generated according to claim 2, wherein each said antisense oligonucleotide further comprises a linker at either 5′-end or 3′-end of said antisense oligonucleotides; wherein said linker being selected from a group consisting antisense sense initiation codons; antisense termination codon; antisense amino acid coding codon; two consecutive antisense codons consisting an antisense restriction enzyme site; and combinations thereof.

19: An antisense oligonucleotide library was generated according to claim 2, wherein said oligonucleotides are grouped according to GC content.

20: An antisense oligonucleotide library was generated according to claim 18, wherein said oligonucleotides are grouped according to GC content.


SEQUENCE LISTING (14 sequences)

SEQ ID NO: 1 - DNA, 42 bp, Artificial
  Synthesized
agcatggccaccatggcagaattcatgtggtaagactagtgc

SEQ ID NO: 2 - DNA, 42 bp, Artificial
  Synthesized
gcactagtcttaccacatgaattctgccatggtggccatgct

SEQ ID NO: 3 - RNA, 48 nt, Artificial
  Synthesized
agcauggccaccauggcagaauucaugugguaagacuagugcaaaaaa

SEQ ID NO: 4 - protein, 6 aa, Artificial
  Synthesized
MAEFMW

SEQ ID NO: 5 - DNA, 24 bp, Artificial
  Synthesized
acgaggactctattatcgccattc

SEQ ID NO: 6 - DNA, 43 bp, Artificial
  sense oligo template
aagaatggcgataatagagtcctctatagtgagtcgtattacc

SEQ ID NO: 7 - DNA, 20 bp, Artificial
  T7 primer
ggtaatacgactcactatag

SEQ ID NO: 8 - RNA, 24 nt, Artificial
  Antisense RNA Oligo
gaggacucuauuaucgccauucuu

SEQ ID NO: 9 - RNA, 24 nt, Artificial
  Sense RNA Oligo
gaauggcgauaauagaguccucgu

SEQ ID NO: 10 - DNA, 48 bp, Artificial
  1st SS cDNA
ttttttgcactagtcttaccacatgaattctgccatggtggccatgct

SEQ ID NO: 11 - DNA, 48 bp, Artificial
  2nd SS cDNA
agcatggccaccatggcagaattcatgtggtaagactagtgcaaaaaa

SEQ ID NO: 12 - DNA, 18 bp, Artificial
  conservative motif of six amino acids of a zinc finger gene family
cacacaggagaaaagcca

SEQ ID NO: 13 - DNA, 18 bp, Artificial
  Antisense conservative motif of six amino acids of a zinc finger gene family
tggcttttctcctgtgtg

SEQ ID NO: 14 - protein, 6 aa, Artificial
  conservative motif of six amino acids of a zinc finger gene family
HTGEFP